(19) United States
(12) Patent Application Publication
(10) Pub. No.: US 2024/0174615 A1
(43) Pub. Date: May 30, 2024

(54) SYNTHESIS OF RAPAMYCIN ANALOG COMPOUNDS

(71) Applicant: Revolution Medicines, Inc., Redwood City, CA (US)

(72) Inventors: Steven G. BALLMER, Redwood City, CA (US); Xiaojun HUANG, Redwood City, CA (US); Shaoling LI, Redwood City, CA (US)

(21) Appl. No.: 18/377,670

(22) Filed: Oct. 6, 2023

Related U.S. Application Data
(63) Continuation of application No. PCT/US2022/023778, filed on Apr. 7, 2022.
(60) Provisional application No. 63/173,189, filed on Apr. 9, 2021.

(51) Int. Cl. C07D 217/04; C07D 487/04; C07D 498/18; C07D 519/00

(52) U.S. Cl. CPC C07D 217/04; C07D 487/04; C07D 498/18; C07D 519/00

(57) ABSTRACT

The present disclosure relates to novel methods for preparing rapamycin analog compounds, as well as to related intermediates useful in such methods.

### CROSS-REFERENCE TO RELATED APPLICATIONS

This application claims the benefit of U.S. Provisional Application No. 63/173,189, filed Apr. 9, 2021, the disclosure of which is hereby incorporated by reference in its entirety.

### FIELD OF THE DISCLOSURE

The present disclosure relates to novel methods for preparing rapamycin analog compounds, as well as to related intermediates useful in such methods.

### BACKGROUND OF THE DISCLOSURE

The mammalian target of rapamycin (mTOR) is a serine-threonine kinase related to the lipid kinases of the phosphoinositide 3-kinase (PI3K) family. mTOR exists in two complexes, mTORC1 and mTORC2, which are differentially regulated, have distinct substrate specificities, and are differentially sensitive to rapamycin. mTORC1 integrates signals from growth factor receptors with cellular nutritional status and controls the level of cap-dependent mRNA translation by modulating the activity of key translational components such as the cap-binding protein and oncogene eIF4E.

mTOR signaling has been deciphered in increasing detail. The differing pharmacology of inhibitors of mTOR has been particularly informative. The first reported inhibitor of mTOR, rapamycin, is now understood to be an incomplete inhibitor of mTORC1. Rapamycin is a selective mTORC1 inhibitor through the binding to the FK506 Rapamycin Binding (FRB) domain of mTOR kinase with the aid of FK506 binding protein 12 (FKBP12). The FRB domain of mTOR is accessible in the mTORC1 complex, but less so in the mTORC2 complex. Interestingly, the potency of inhibitory activities against downstream substrates of mTORC1 by the treatment of rapamycin is known to be diverse among the mTORC1 substrates. For example, rapamycin strongly inhibits phosphorylation of the mTORC1 substrate S6K and, indirectly, phosphorylation of the downstream ribosomal protein S6 which control ribosomal biogenesis. On the other hand, rapamycin shows only partial inhibitory activity against phosphorylation of 4E-BP1, a major regulator of eIF4E which controls the initiation of CAP-dependent translation. As a result, more complete inhibitors of mTORC1 signaling are of interest.

A second class of “ATP-site” inhibitors of mTOR kinase were reported. The molecules compete with ATP, the substrate for the kinase reaction, in the active site of the mTOR kinase (and are therefore also mTOR active site inhibitors). As a result, these molecules inhibit downstream phosphorylation of a broader range of substrates.

Although mTOR inhibition may have the effect of blocking 4E-BP1 phosphorylation, these agents may also inhibit mTORC2, which leads to a block of Akt activation due to inhibition of phosphorylation of Akt S473.

In order to accelerate the drug discovery and development process, new methods for synthesizing rapamycin analogs are needed to provide an array of compounds that are potentially new drugs. The present disclosure fulfills these needs and provides further related advantages.

### SUMMARY OF THE DISCLOSURE

In brief, the present disclosure relates to novel methods for preparing rapamycin analog compounds and novel intermediates used in the new methods.

The present disclosure provides processes for preparing a compound of formula (33) that are scaleable and reproducible at a commercial scale. These processes comprise reactions that can provide novel intermediate compounds obtained through experimentation and development of new combinations of reaction conditions.

One aspect of the disclosure relates to a process for preparing a compound of formula (3), or a salt thereof, comprising: step (1a) contacting a compound of formula (1), or a salt thereof,

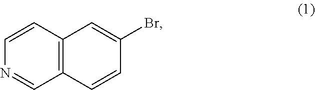 [SMILES: Brc1ccc2cnccc2c1]

with a reducing agent, to yield a compound of formula (2), or a salt thereof,

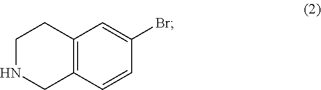 [SMILES: Brc1ccc2c(c1)CCNC2]

step (2a) contacting a compound of formula (2), or a salt thereof, with an amino protecting group reagent to yield a compound of formula (3), or a salt thereof,

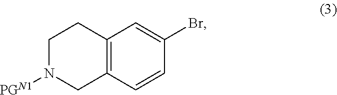

wherein PG^{N1} is an amino protecting group. In certain embodiments, the reducing agent is sodium borohydride. In certain embodiments, step (1a) is performed in the presence of acetic acid. In certain embodiments, the amino protecting group reagent is triphenylmethyl chloride. In certain embodiments, PG^{N1} is triphenylmethyl (trityl). In certain embodiments, step (2a) is performed in the presence of an activating reagent. In certain embodiments, the activating reagent is 4-dimethylaminopyridine (DMAP). In certain embodiments, step (2a) is performed in dichloromethane (DCM). In certain embodiments, the method further comprises isolating the compound of formula (3).

Another aspect of the disclosure relates to a process further comprising step (3a′) contacting the compound of formula (3), or a salt thereof, with an organometallic/metal reagent and formaldehyde to yield a compound of formula (5), or a salt thereof,

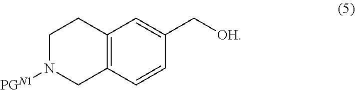

In certain embodiments, the organometallic/metal reagent is magnesium. In certain embodiments, step (3a) is performed in tetrahydrofuran (THF).

Another aspect of the disclosure relates to a process further comprising step (3a) contacting the compound of formula (3), or a salt thereof, with an organometallic reagent and dimethylformamide (DMF) to yield a compound of formula (4), or a salt thereof,

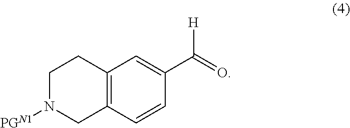

In certain embodiments, the organometallic reagent is an alkyl magnesium halide. In certain embodiments, step (3a) is performed in tetrahydrofuran (THF).

Another aspect of the disclosure relates to a process further comprising step (4a) contacting the compound of formula (4), or a salt thereof, with a reducing agent to yield a compound of formula (5), or a salt thereof,

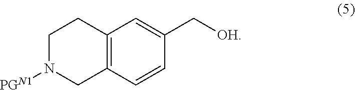

In certain embodiments, the reducing agent is sodium borohydride. In certain embodiments, step (4a) is performed in a solvent selected from the group consisting of methanol, THE, and mixture thereof.

Another aspect of the disclosure relates to a process further comprising step (5a) contacting the compound of formula (5), or a salt thereof, with a PG^{N1} deprotecting reagent to yield a compound of formula (6), or a salt thereof,

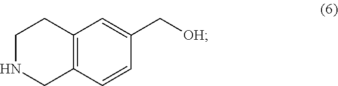 [SMILES: OCc1ccc2c(c1)CCNC2]

step (6a) contacting the compound of formula (6), or a salt thereof, with a Boc protecting group reagent to yield a compound of formula (7), or a salt thereof,

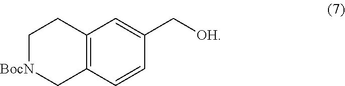 [SMILES: CC(C)(C)OC(=O)N1CCc2cc(CO)ccc2C1]

In certain embodiments, the PG^{N1} deprotecting reagent is an acid. In certain embodiments, step (5a) is performed in DCM. In certain embodiments, the Boc protecting group reagent is Boc₂O. In certain embodiments, step (6a) is performed in THF. In certain embodiments, the method further comprises isolating the compound of formula (7).

Another aspect of the disclosure relates to a process further comprising step (7a) contacting the compound of formula (7), or a salt thereof, with an alcohol activating reagent to yield a compound of formula (8), or a salt thereof,

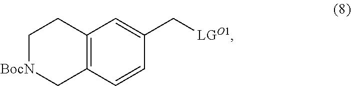

wherein −LG^{O1} is a leaving group. In certain embodiments, the alcohol activating reagent is a sulfonyl halide or a halogenating reagent. In certain embodiments, the alcohol activating reagent is methanesulfonyl chloride (mesyl chloride; CH₃SO₂Cl). In certain embodiments, -LG^{O1} is a sulfonate ester or a halide. In certain embodiments, −LG^{O1} is mesylate (—O—SO₂CH₃). In certain embodiments, step (7a) is performed in the presence of a base. In certain embodiments, the base is diisopropylethylamine (DIPEA). In certain embodiments, step (7a) is performed in DCM. In certain embodiments, the method further comprises isolating the compound of formula (8).

Another aspect of the disclosure relates to a process further comprising step (8a) contacting the compound of formula (8), or a salt thereof, with a compound of formula (9), or a salt thereof,

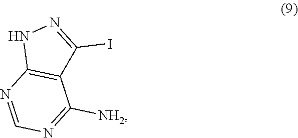 [SMILES: Nc1ncnc2[nH]nc(I)c12]

to yield a compound of formula (10), or a salt thereof,

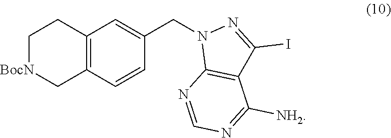 [SMILES: CC(C)(C)OC(=O)N1CCc2cc(Cn3nc(I)c4c(N)ncnc43)ccc2C1]

In certain embodiments, step (8a) is performed in DMF. In certain embodiments, the method further comprises isolating the compound of formula (10).

Another aspect of the disclosure relates to a process further comprising step (9a) contacting the compound of formula (10), or a salt thereof, with a compound of formula (11) or a salt thereof,

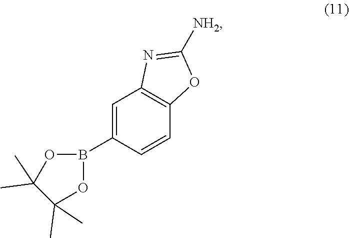 [SMILES: CC1(C)OB(c2ccc3oc(N)nc3c2)OC1(C)C]

to yield a compound of formula (12), or a salt thereof,

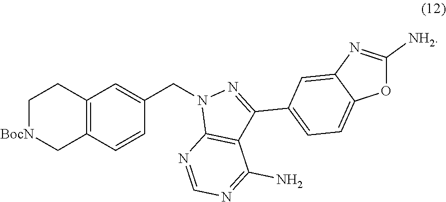 [SMILES: CC(C)(C)OC(=O)N1CCc2cc(Cn3nc(-c4ccc5oc(N)nc5c4)c4c(N)ncnc43)ccc2C1]

In certain embodiments, the compound of formula (11) is prepared by borylation of a compound of formula (11a), or a salt thereof,

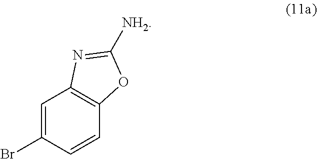 [SMILES: Nc1nc2cc(Br)ccc2o1]

In certain embodiments, the borylation is performed with contact with a boronic ester reagent. In certain embodiments, the boronic ester reagent is bis(pinacolato)diboron (B₂Pin₂). In certain embodiments, step (9a) is performed in the presence of a palladium catalyst. In certain embodiments, the palladium catalyst is Pd(PPh₃)₄. In certain embodiments, step (9a) is performed in a solvent selected from the group consisting of water, dioxane, and mixture thereof. In certain embodiments, the method further comprises isolating the compound of formula (12).

Another aspect of the disclosure relates to a process further comprising step (10a) contacting the compound of formula (12) with an acid to yield a compound of formula (13),

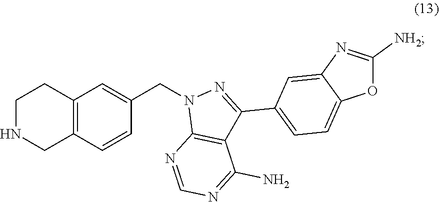 [SMILES: Nc1nc2cc(-c3nn(Cc4ccc5c(c4)CCNC5)c4ncnc(N)c34)ccc2o1]

step (11a) preparing a salt of a compound of formula (13). In certain embodiments, the acid is hydrochloric acid, thereby yielding a hydrochloric salt of compound of formula (13a),

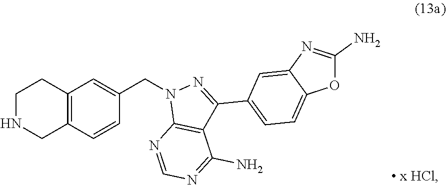

wherein x is 1, 2, or 3. In certain embodiments, x is 3. In certain embodiments, the acid is trifluoroacetic acid, thereby yielding a TFA salt of compound of formula (13c),

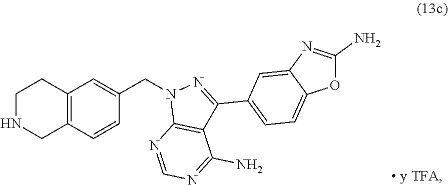

wherein y is 1, 2, or 3. In certain embodiments, y is 3. In certain embodiments, step (10a) and step (11a) are performed in water. In certain embodiments, the method further comprises isolating the compound of formula (13), (13a), or (13c).

One aspect of the disclosure relates to a process for preparing a compound of formula (21), or a salt thereof, comprising: step (1b) contacting a compound of formula (20), or a salt thereof,

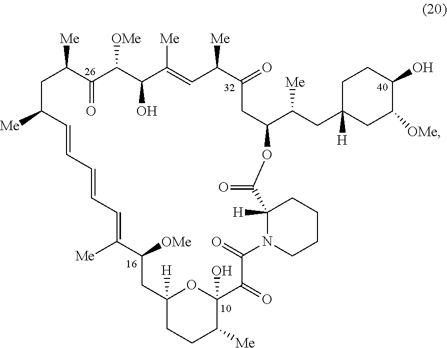 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

with a hydroxyl protecting group reagent, to yield a compound of formula (21), or a salt thereof,

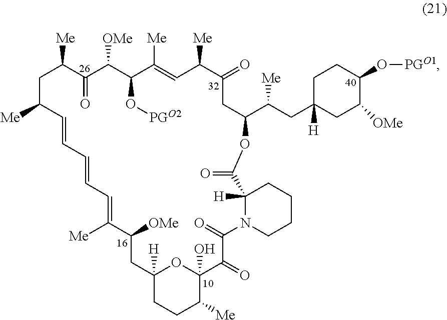

wherein PG^{O1} and PG^{O2} are the same or different at each instance a hydroxyl protecting group. In certain embodiments, each hydroxyl protecting group reagent is triethylchlorosilane (TES-C1) In certain embodiments, PG^{O1} is triethylsilyl ether (TES). In certain embodiments, PG^{O2} is triethylsilyl ether (TES). In certain embodiments, step (1b) is performed in the presence of imidazole. In certain embodiments, step (1b) is performed in dichloromethane. In certain embodiments, the method further comprises isolating the compound of formula (21).

Another aspect of the disclosure relates to a process further comprising: step (2b) contacting a compound of formula (21), or a salt thereof, with a reducing agent to yield a compound of formula (22), or a salt thereof,

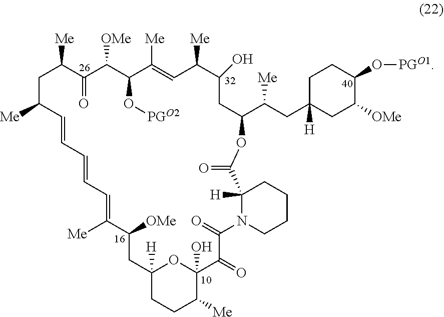

In certain embodiments, the reducing agent is LiAl(Ot-Bu)₃H. In certain embodiments, the product from step (2b) is subsequently contacted with Cu(OAc)₂. In certain embodiments, step (2b) is performed in THIF. In certain embodiments, the method further comprises isolating the compound of formula (22).

Another aspect of the disclosure relates to a process further comprising: step (3b) contacting a compound of formula (22), or a salt thereof, with a PG^{O1} deprotecting reagent and a PG^{O2} deprotecting reagent to yield a compound of formula (23), or a salt thereof,

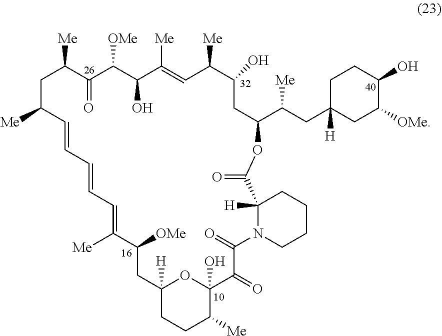 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In certain embodiments, the PG^{O1} deprotecting reagent is an acid. In certain embodiments, the PG^{O2} deprotecting reagent is an acid. In certain embodiments, step (3b) is performed in THF. In certain embodiments, the method further comprises isolating the compound of formula (23).

Another aspect of the disclosure relates to a process further comprising step (4b) contacting the compound of formula (23), or a salt thereof, with a compound of formula (24), or a salt thereof,

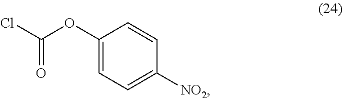 [SMILES: O=C(Cl)Oc1ccc([N+](=O)[O-])cc1]

to yield a compound of formula (25), or a salt thereof,

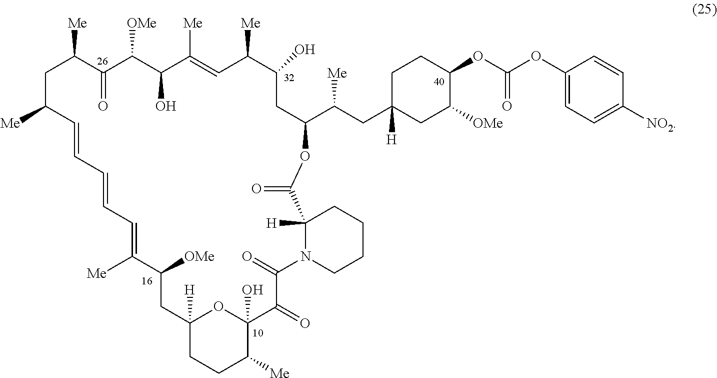 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In certain embodiments, step (4b) is performed in DCM. In certain embodiments, the method further comprises isolating the compound of formula (25).

One aspect of the disclosure relates to a process for preparing a compound of formula (31), or a salt thereof, comprising. step (10c) contacting a compound of formula (30), or a salt thereof,

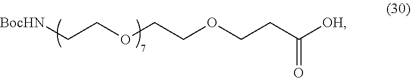

with a compound of formula (13), or a salt thereof,

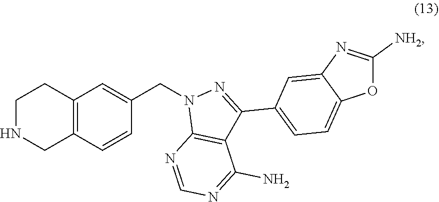 [SMILES: Nc1nc2cc(-c3nn(Cc4ccc5c(c4)CCNC5)c4ncnc(N)c34)ccc2o1]

to yield the compound of formula (31), or a salt thereof,

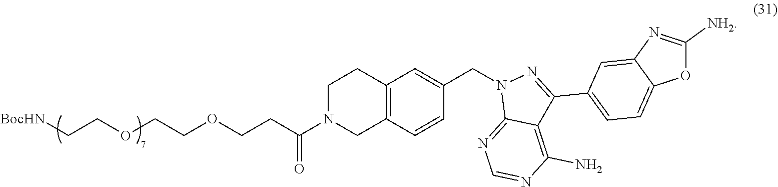

In certain embodiments, the compound of formula (13), or a salt thereof, is a compound of formula (13a),

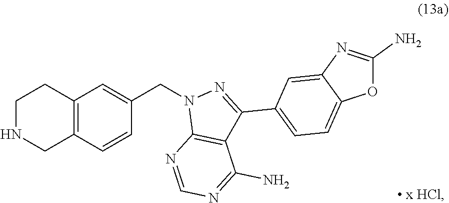

wherein n is 1, 2, or 3. In certain embodiments, x is 3. In certain embodiments, the compound of formula (13), or a salt thereof, is a compound of formula (13c),

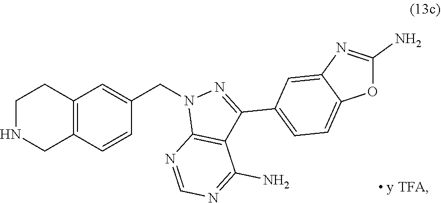

wherein y is 1, 2, or 3. In certain embodiments, y is 3. In certain embodiments, step (1c) is performed in the presence of a coupling reagent. In certain embodiments, the coupling reagent is 1-ethyl-3-(3-dimethylaminopropyl)carbodiimide (EDCI). In certain embodiments, step (1c) is performed in the presence of an activating reagent. In certain embodiments, the activating reagent is hydroxybenzotriazole (HOBt). In certain embodiments, step (1c) is performed in dimethylacetamide (DIMM). In certain embodiments, the method further comprises isolating the compound of formula (31).

Another aspect of the disclosure relates to a process further comprising step (2c) contacting the compound of formula (31) with a Boc removing agent to yield a compound of formula (32), or a salt thereof,

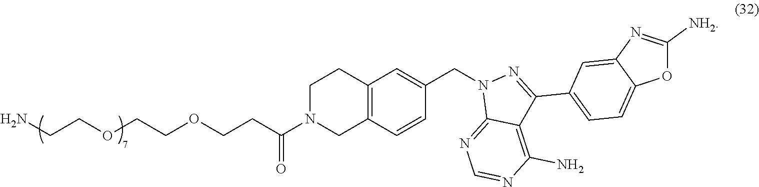

In certain embodiments, the Boc removing reagent is hydrochloric acid. In certain embodiments, step (2c) is performed in a solvent selected from the group consisting of water, dichloromethane, dimethylacetamide (DMAc), and mixtures thereof. In certain embodiments, the method further comprises isolating the compound of formula (32).

Another aspect of the disclosure relates to a process further comprising step (3c) contacting the compound of formula (32), or a salt thereof, with a compound of formula (25), or a salt thereof,

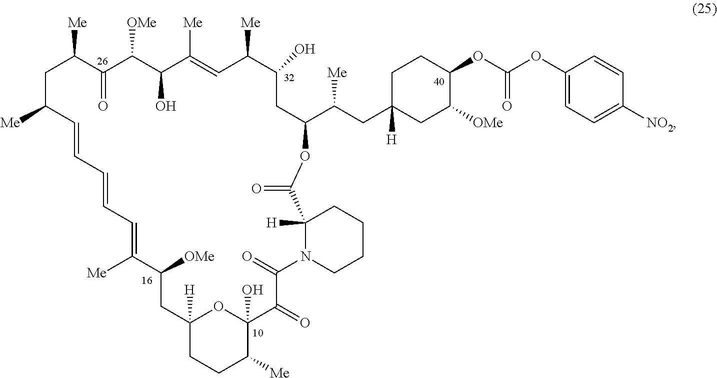 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

to yield a compound of formula (33), or a salt thereof,

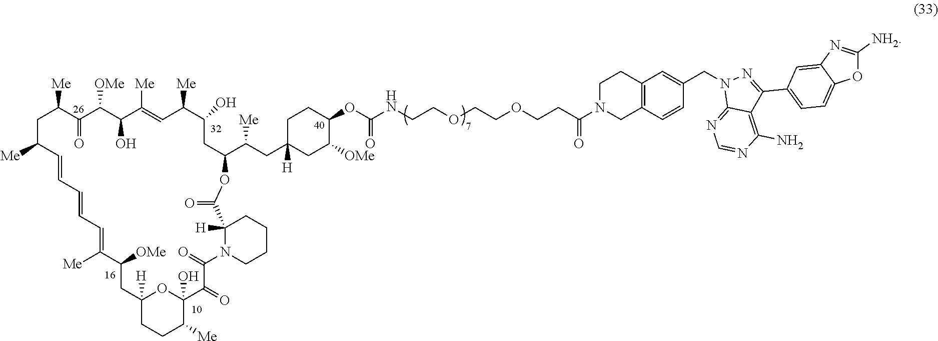

In certain embodiments, step (3c) is performed in dimethylacetamide (DMAc). In certain embodiments, the method further comprises isolating the compound of formula (33).

One aspect of the disclosure relates to compound of formula (13), or a salt thereof.

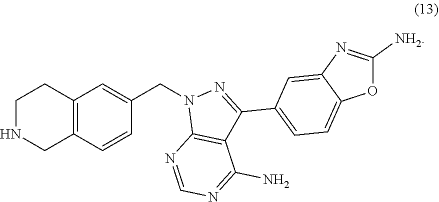 [SMILES: Nc1nc2cc(-c3nn(Cc4ccc5c(c4)CCNC5)c4ncnc(N)c34)ccc2o1]

One aspect of the disclosure relates to compound of formula (13a):

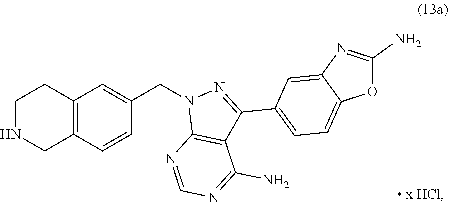

wherein x is 1, 2, or 3. In certain embodiments, x is 3:

One aspect of the disclosure relates to compound of formula (13b):

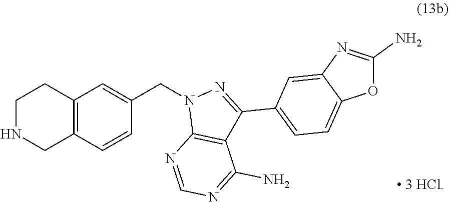 [SMILES: Nc1nc2cc(-c3nn(Cc4ccc5c(c4)CCNC5)c4ncnc(N)c34)ccc2o1]

One aspect of the disclosure relates to compound of formula (13c):

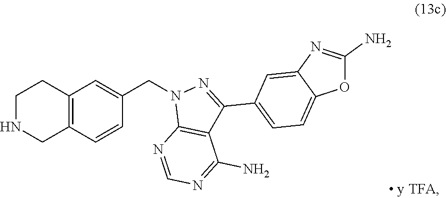

wherein y is 1, 2, or 3. In certain embodiments, y is 3:

One aspect of the disclosure relates to compound of formula (13d):

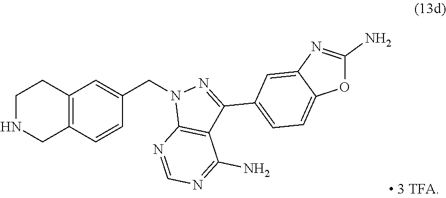 [SMILES: Nc1nc2cc(-c3nn(Cc4ccc5c(c4)CCNC5)c4ncnc(N)c34)ccc2o1]

One aspect of the disclosure relates to compound of formula (32), or a salt thereof:

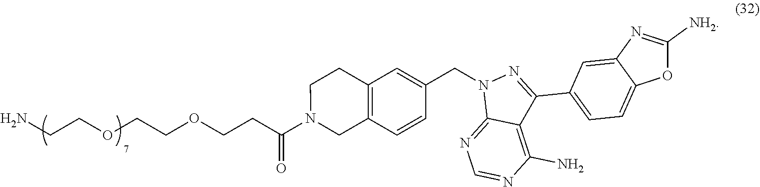

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

Each embodiment described herein may be taken alone or in combination with any one or more other embodiments.

### DETAILED DESCRIPTION OF THE DISCLOSURE

The present disclosure relates to novel methods for preparing rapamycin analog compounds, as well as to related intermediates useful in such methods.

As discussed herein, the present disclosure provides processes for preparing a compound of formula (33) that is scaleable and reproducible at commercial scale. The processes comprise combinations of reactions and conditions that can provide certain novel intermediate compounds.

In one aspect, the disclosure relates to a process for preparing a compound of formula (3), or a salt thereof,

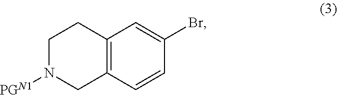

wherein PG^{N1} is an amino protecting group.

In one aspect, the disclosure relates to a process for preparing a compound of formula (4), or a salt thereof,

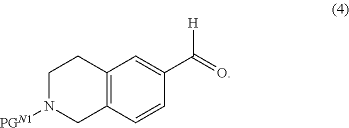

In one aspect, the disclosure relates to a process for preparing a compound of formula (5), or a salt thereof,

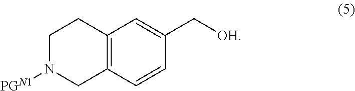

In one aspect, the disclosure relates to a process for preparing a compound of formula (6), or a salt thereof,

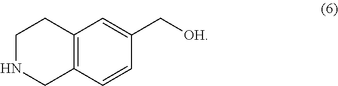 [SMILES: OCc1ccc2c(c1)CCNC2]

In one aspect, the disclosure relates to a process for preparing a compound of formula (7), or a salt thereof,

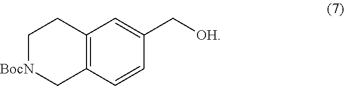 [SMILES: CC(C)(C)OC(=O)N1CCc2cc(CO)ccc2C1]

In one aspect, the disclosure relates to a process for preparing a compound of formula (8), or a salt thereof,

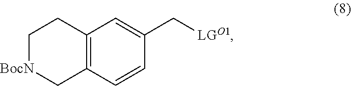

wherein −LG^{O1} is a leaving group.

In one aspect, the disclosure relates to a process for preparing a compound of formula (9), or a salt thereof,

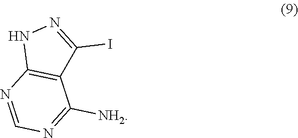 [SMILES: Nc1ncnc2[nH]nc(I)c12]

In one aspect, the disclosure relates to a process for preparing a compound of formula (10), or a salt thereof,

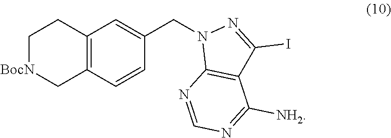 [SMILES: CC(C)(C)OC(=O)N1CCc2cc(Cn3nc(I)c4c(N)ncnc43)ccc2C1]

In one aspect, the disclosure relates to a process for preparing a compound of formula (11), or a salt thereof,

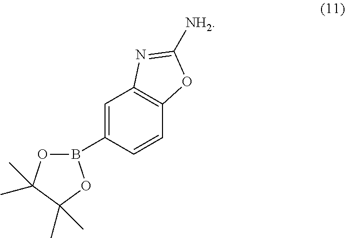 [SMILES: CC1(C)OB(c2ccc3oc(N)nc3c2)OC1(C)C]

In one aspect, the disclosure relates to a process for preparing a compound of formula (12), or a salt thereof,

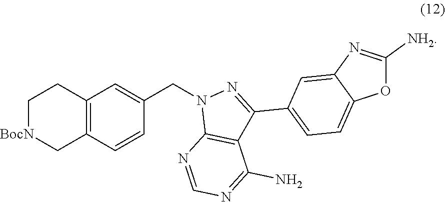 [SMILES: CC(C)(C)OC(=O)N1CCc2cc(Cn3nc(-c4ccc5oc(N)nc5c4)c4c(N)ncnc43)ccc2C1]

In one aspect, the disclosure relates to a process for preparing a compound of formula (13), or a salt thereof,

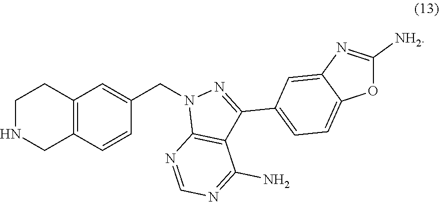 [SMILES: Nc1nc2cc(-c3nn(Cc4ccc5c(c4)CCNC5)c4ncnc(N)c34)ccc2o1]

In one aspect, the disclosure relates to a process for preparing a compound of formula (13a),

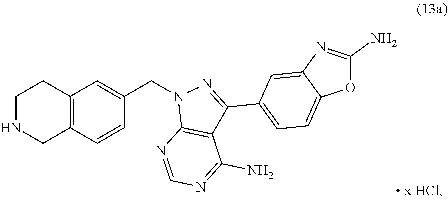

wherein x is 1, 2, or 3. In certain embodiments, x is 3, shown as a compound of formula (13b),

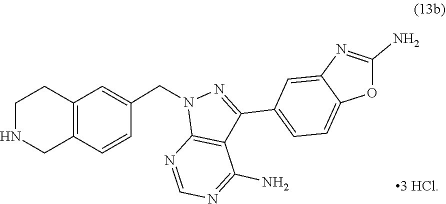 [SMILES: Nc1nc2cc(-c3nn(Cc4ccc5c(c4)CCNC5)c4ncnc(N)c34)ccc2o1]

In one aspect, the disclosure relates to a process for preparing a compound of formula (13c),

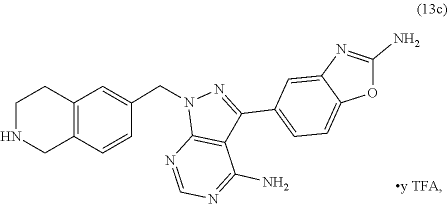

wherein y is 1, 2, or 3. In certain embodiments, y is 3, shown as a compound of formula (13d),

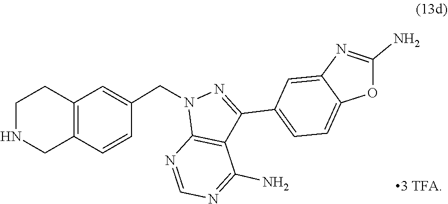 [SMILES: Nc1nc2cc(-c3nn(Cc4ccc5c(c4)CCNC5)c4ncnc(N)c34)ccc2o1]

In one aspect, the disclosure relates to a process for preparing a compound of formula (21), or a salt thereof,

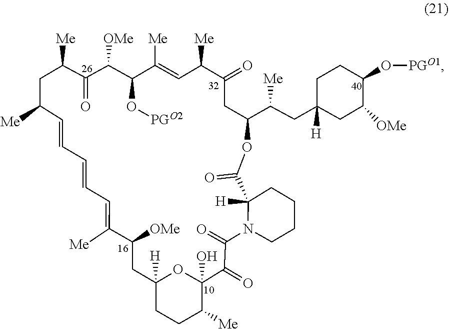

wherein PG^{O1} and PG^{O2} are independently same or different hydroxyl protecting groups.

In one aspect, the disclosure relates to a process for preparing a compound of formula (22), or a salt thereof,

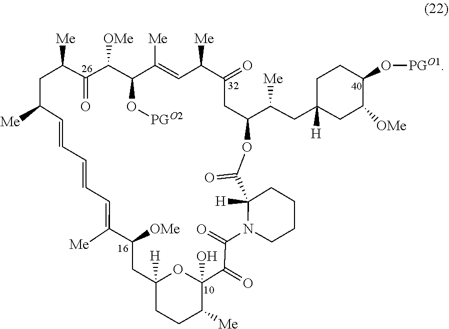

In one aspect, the disclosure relates to a process for preparing a compound of formula (23), or a salt thereof,

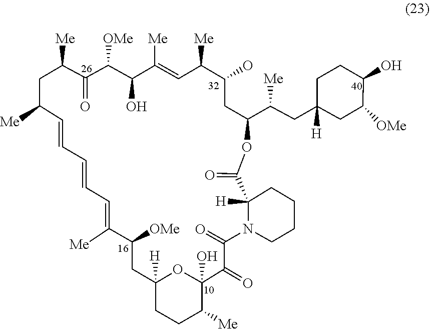 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In one aspect, the disclosure relates to a process for preparing a compound of formula (25), or a salt thereof,

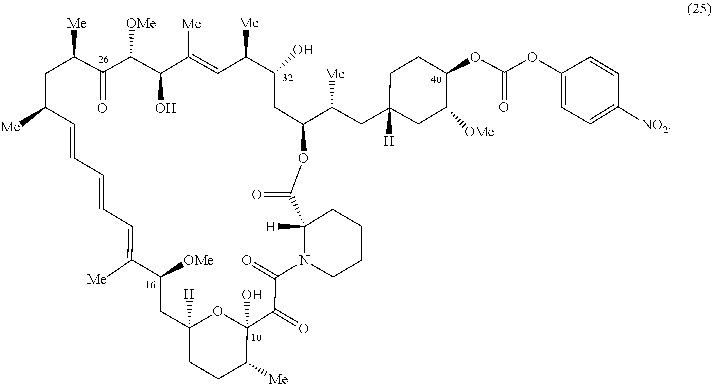 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In one aspect, the disclosure relates to a process for preparing a compound of formula (31), or a salt thereof,

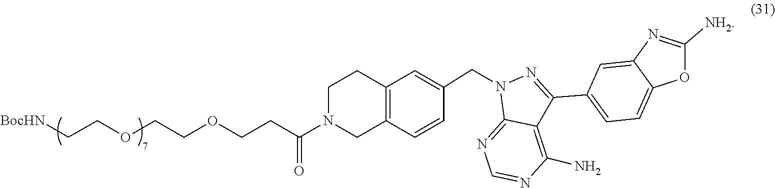

In one aspect, the disclosure relates to a process for preparing a compound of formula (32), or a salt thereof,

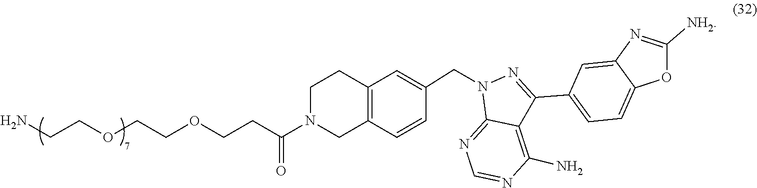

In one aspect, the disclosure relates to a process for preparing a compound of formula (33), or a salt thereof,

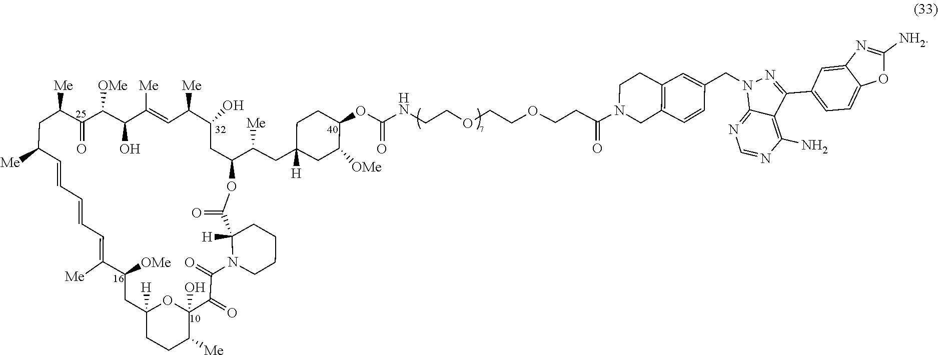

In one aspect, the disclosure relates to a compound of formula (13), or a salt thereof.

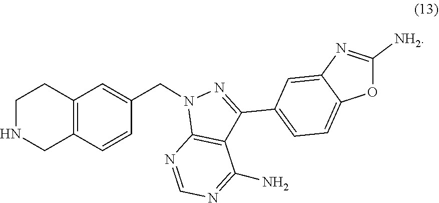 [SMILES: Nc1nc2cc(-c3nn(Cc4ccc5c(c4)CCNC5)c4ncnc(N)c34)ccc2o1]

In one aspect, the disclosure relates to a compound of formula (13a):

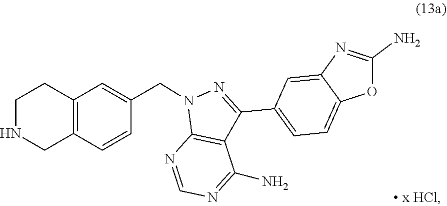

wherein x is 1, 2, or 3. In certain embodiments, x is 3, shown as a compound of formula (13b),

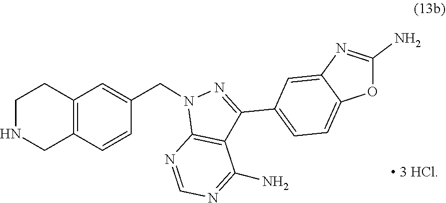 [SMILES: Nc1nc2cc(-c3nn(Cc4ccc5c(c4)CCNC5)c4ncnc(N)c34)ccc2o1]

In one aspect, the disclosure relates to a compound of formula (13c):

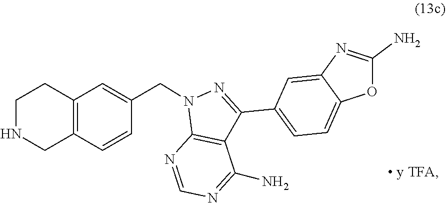

wherein y is 1, 2, or 3. In certain embodiments, y is 3, shown as a compound of formula (13d),

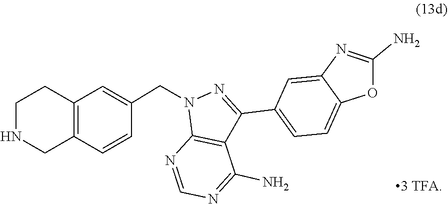 [SMILES: Nc1nc2cc(-c3nn(Cc4ccc5c(c4)CCNC5)c4ncnc(N)c34)ccc2o1]

In one aspect, the disclosure relates to a compound of formula (32), or a salt thereof:

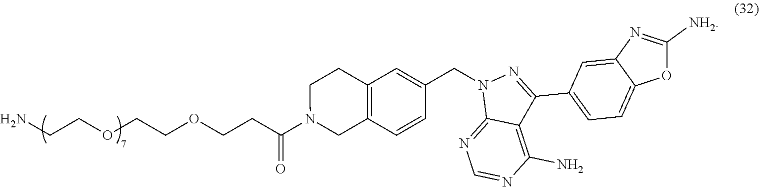

### Terms and Abbreviations:

The articles “a” and “an” as used in this disclosure may refer to one or more than one (i.e., to at least one) of the grammatical object of the article. By way of example, “an element” means one element or more than one element.

As used herein, the term “about” may be used to indicate that a value includes the standard deviation of error for the device or method being employed to determine the value. In certain embodiments, the term “about” refers to a range of values that fall within 25%, 20%, 19%, 18%, 17%, 16%, 1, 14%, 13%, 12%, 11%, 10%, 9%, 8%, 7%, 6%, 5%, 4%, 3%, 2%, 1%, or less in either direction (greater than or less than) of a stated value, unless otherwise stated or otherwise evident from the context (e.g., where such number would exceed 100% of a possible value).

As used in this disclosure, “and/or” may mean either “and” or “or” unless indicated otherwise.

“Alkyl” may refer to a straight or branched chain saturated hydrocarbon. C₁-C₃alkyl groups contain 1 to 3 carbon atoms. Examples of a C₁-C₃alkyl group include, but are not limited to, methyl, ethyl, and propyl.

The term “protecting group,” as used herein, may refer to a labile chemical moiety which is known in the art to protect reactive groups including without limitation, hydroxyl and amino groups, against undesired reactions during synthetic procedures. Hydroxyl and amino groups which protected with a protecting group are referred to herein as “protected hydroxyl groups” and “protected amino groups”, respectively. Protecting groups are typically used selectively or orthogonally to protect sites during reactions at other reactive sites and can then be removed to leave the unprotected group as is or available for further reactions. Protecting groups as known in the art are described generally in Greene and Wuts, Protective Groups in Organic Synthesis, 3rd edition, John Wiley & Sons, New York (1999). Groups may be selectively incorporated into aminoglycosides described herein as precursors. For example, an amino group can be placed into a compound described herein as an azido group that can be chemically converted to the amino group at a desired point in the synthesis. Generally, groups are protected or present as a precursor that will be inert to reactions that modify other areas of the parent molecule for conversion into their final groups at an appropriate time. Further, representative protecting or precursor groups are discussed in Agrawal, et al., Protocols for Oligonucleotide Conjugates, Eds, Humana Press; New Jersey, 1994; Vol. 26 pp. 1-72. Examples of “hydroxyl protecting groups” include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p-nitrobenzyl, triphenylmethyl, trimethylsilyl, triethylsilyl, t-butyldimethylsilyl, t-butyldiphenylsilyl (TBDPS), triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate. Examples of “amino protecting groups” include, but are not limited to, triphenylmethyl (trityl; Trt), 2-trimethylsilylethoxycarbonyl (Teoc), 1-methyl-1-(4-biphenylyl)ethoxycarbonyl (Bpoc), t-butoxycarbonyl (Boc), allyloxycarbonyl (Alloc), 9-fluorenylmethyloxycarbonyl (Fmoc), benzyloxycarbonyl (Cbz), p-nitrobenzyloxycarbonyl (PNZ), formyl, acetyl, trihaloacetyl (e.g., trifluoroacetyl), benzoyl, nitrophenylacetyl, 2-nitrobenzenesulfonyl, phthalimido, and dithiasuccinoyl.

“Boc protecting group reagent” may refer to a reagent that may be used to install a Boc protecting group on an amine group. Examples of Boc protecting group reagents include, but are not limited to, Boc anhydride (Boc₂O), N-tert-butoxycarbonylimidazole, 2-(tert-butoxycarbonyloxyimino)-2-phenylacetonitrile, 2-(tert-butoxycarbonylthio)-4,6-dimethylpyrimidine, 1-tert-butoxycarbonyl-1,2,4-triazole, tert-butyl phenyl carbonate, N-(tert-butoxycarbonyloxy)phthalimide, tert-butyl 2,4,5-trichlorophenyl carbonate, and tert-butyl ((4R,7S)-1,3-dioxo-1,3,3a,4,7,7a-hexahydro-2H-4,7-methanoisoindol-2-yl) carbonate (Boc-ONb).

“Boc removing reagent” may refer to a reagent that may be used to cleave a Boc protecting group on an amine group. Examples of Boc removing reagents include, but are not limited to, TFA, aqueous phosphoric acid, methanesulfonic acid (MSA), SnCl₄, HCl, HCl/dioxane, and HCl/MeOH.

### Preparation of a Compound of Formula (33) and Intermediates Thereof

The present disclosure includes processes, methods, reagents, and intermediates for the synthesis of a compound of formula (33), or a salt thereof, which has the structure:

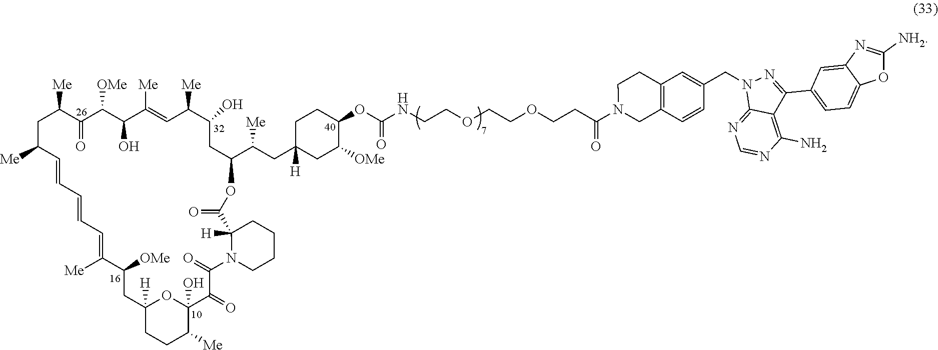

Conventional atom numbering for rapamycin is shown below:

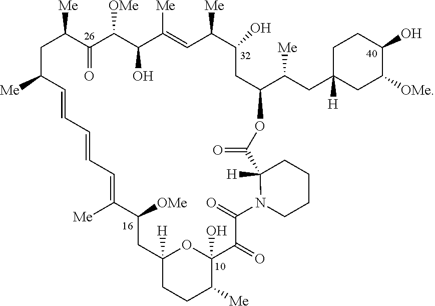 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

A process for the preparation of a compound of formula (33) and certain intermediates obtained in the preparation of a compound of formula (33) is illustrated in Schemes 103 below and is discussed in greater detail herein.

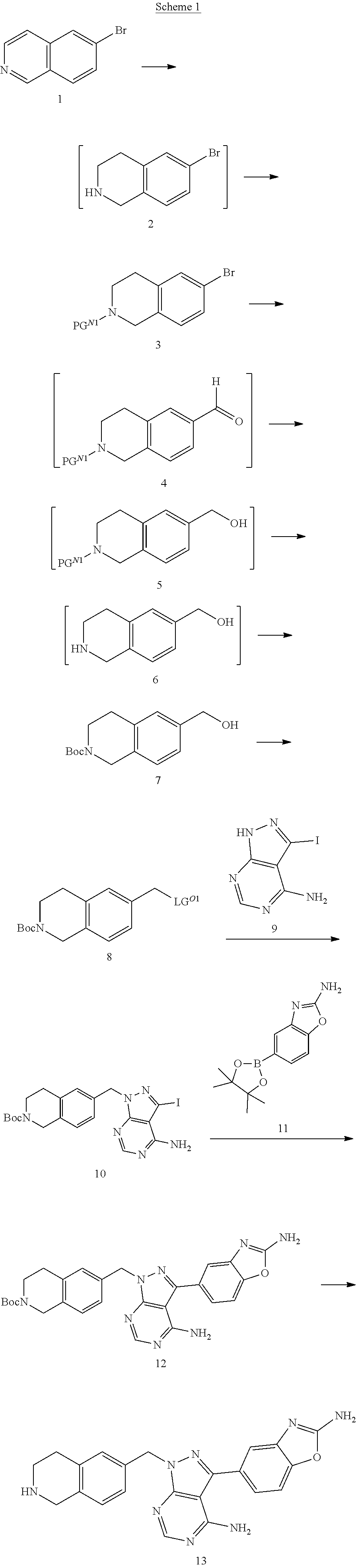

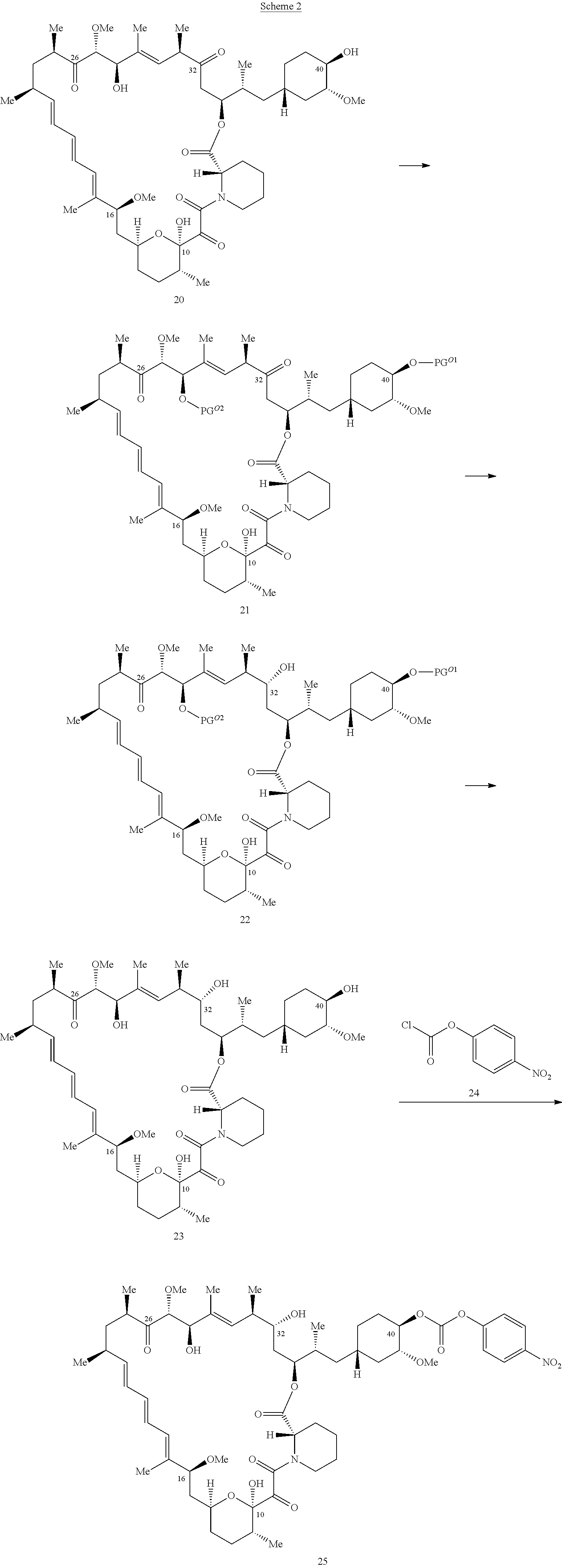

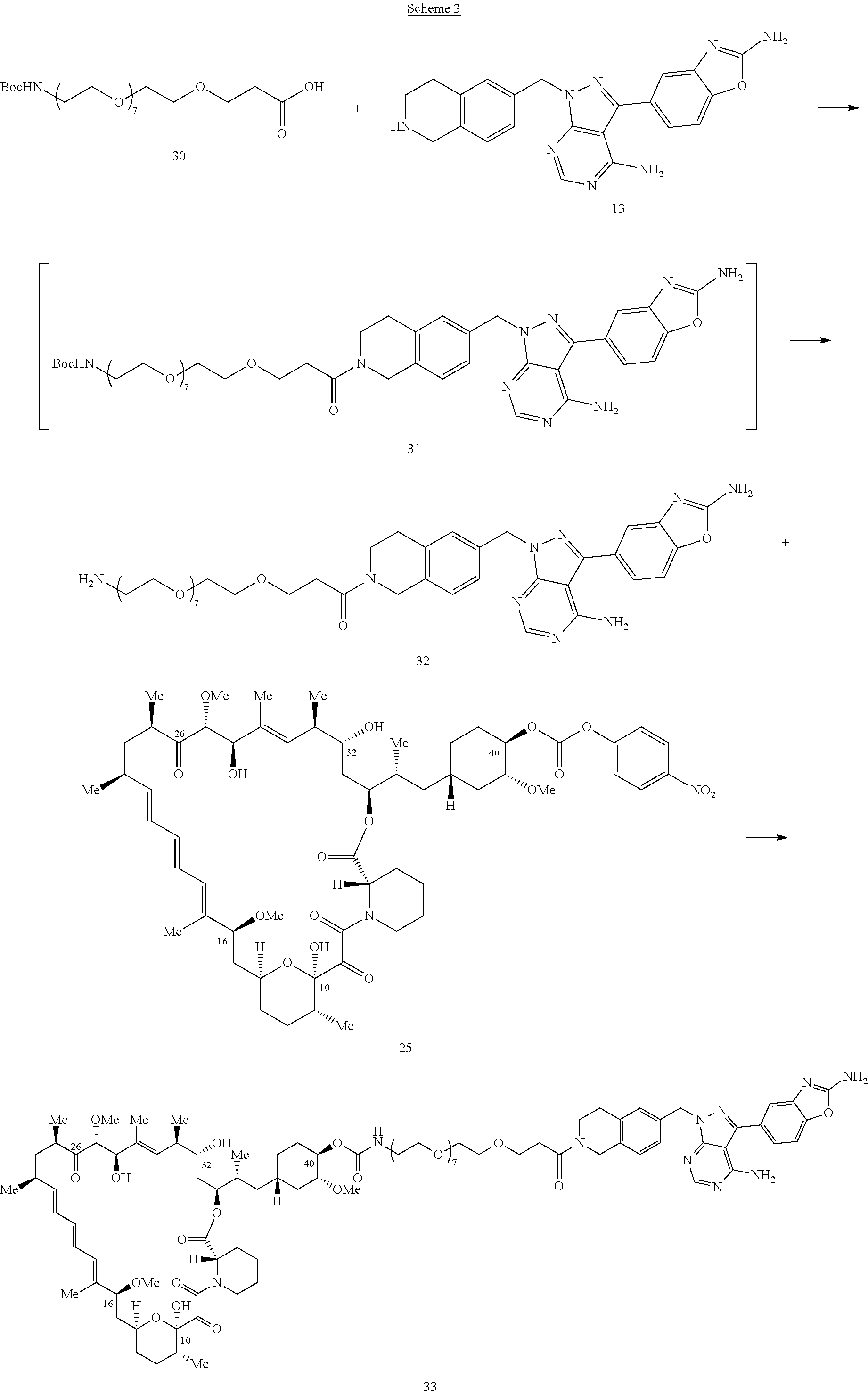

As noted above, the present disclosure provides processes for preparing a compound of formula (33) that is not only scaleable to large quantities, but which is also reproducible batch to batch at a large scale. In some embodiments, the synthetic methods and purification processes described herein outline a scaleable process for the preparation of compounds of formula (33), and intermediates thereof, which does not rely on elaborate steps during the preparation, thus making this methodology amenable to large scale production of rapamycin analogs. Another advantage is that the use of purification columns is reduced.

The compounds described herein and the process of making the compounds may include salts of the compounds described herein. Representative salts include, but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

A salt may also include acid addition salts. An “acid addition salt” may refer to those salts which retain the biological effectiveness and properties of the freebases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, l-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

The compounds described herein and the process of making the compounds may include solvates of the compounds described herein. The term “solvate” may refer to a complex of variable stoichiometry formed by a solute and solvent. Such solvents for the purpose of the disclosure may not interfere with the biological activity of the solute. Examples of suitable solvents may include, but are not limited to, water, MeOH, EtOH, and AcOH. Solvates wherein water is the solvent molecule are typically referred to as hydrates. Hydrates may include compositions containing stoichiometric amounts of water, as well as compositions containing variable amounts of water.

Those skilled in the art will recognize if a stereocenter exists in any of the compounds described herein and the process of making the compounds. Accordingly, the present disclosure includes both possible stereoisomers (unless the stereochemistry is specified herein) and includes not only racemic compounds but the individual enantiomers or diastereomers as well. When a compound is desired as a single enantiomer or diastereomer, it may be obtained by stereospecific synthesis or by resolution of the final product or any convenient intermediate. Resolution of the final product, an intermediate, or a starting material may be effected by any suitable method known in the art. See, for example, “Stereochemistry of Organic Compounds” by E. L. Eliel, S. H. Wilen, and L. N. Mander (Wiley-Interscience, 1994).

The term “stereoisomers” may refer to the set of compounds which have the same number and type of atoms and share the same bond connectivity between those atoms, but differ in three dimensional structure. The term “stereoisomer” may refer to any member of this set of compounds. For instance, a stereoisomer may be an enantiomer or a diastereomer. The compounds described herein and the process of making the compounds may include stereoisomers.

The term “enantiomers” may refer to a pair of stereoisomers which are non-superimposable mirror images of one another. The term “enantiomer” may refer to a single member of this pair of stereoisomers. The term “racemic” may refer to a 1:1 mixture of a pair of enantiomers. The compounds described herein and the process of making the compounds may include enantiomers. Each compound herein disclosed may include all the enantiomers that conform to the general structure of the compound (unless the enantiomer is specified herein). The compounds may be in a racemic or enantiomerically pure form, or any other form in terms of stereochemistry (unless the stereochemistry is specified herein). In some embodiments the compounds are the (S)-enantiomer. In other embodiments the compounds are the (R)-enantiomer. In yet other embodiments, the compounds are the (+) or (−) enantiomers. In some embodiments, compounds described herein may be enriched to provide predominantly one enantiomer of a compound described herein. An enantiomerically enriched mixture may comprise, for example, at least 60 mol percent of one enantiomer, or more preferably at least 75, 80, 85, 90, 95, 96, 97, 98, 99, 99.5 or even 100 mol percent. In some embodiments, the compound described herein enriched in one enantiomer may be substantially free of the other enantiomer, wherein substantially free means that the substance in question makes up less than 10%, or less than 5%, or less than 4%, or less than 3%, or less than 2%, or less than 1% as compared to the amount of the other enantiomer, e.g., in the compound mixture. For example, if a compound mixture contains 98 grams of a first enantiomer and 2 grams of a second enantiomer, it would be said to contain 98 mol percent of the first enantiomer and only 2 mol percent of the second enantiomer.

The term “diastereomers” may refer to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” may refer to any member of this set of compounds. In some examples presented, the synthetic route may produce a single diastereomer or a mixture of diastereomers. The compounds described herein and the process of making the compounds may include diastereomers. In some embodiments, the compounds described herein may be enriched to provide predominantly one diastereomer of a compound disclosed herein. A diastereomerically enriched mixture may comprise, for example, at least 60 mol percent of one diastereomer, or more preferably at least 75, 99, 95, 96, 97, 98, 99, or even 100 mol percent.

In addition, the compounds described herein and the process of making the compounds include all geometric and positional isomers. For example, if a compound described herein incorporates a double bond or a fused ring, both the cis- and trans-forms, as well as mixtures, may be embraced within the scope of the disclosure. If the compound contains a double bond, the substituent may be in the E or Z configuration (unless the configuration is specified herein). If the compound contains a disubstituted cycloalkyl, the cycloalkyl substituent may have a cis or trans configuration (unless the configuration is specified herein).

The compounds described herein may further include all isotopically labeled compounds. An “isotopically” or “radio-labeled” compound is a compound where one or more atoms are replaced or substituted by an atom having an atomic mass or mass number different from the atomic mass or mass number typically found in nature (i.e., naturally occurring). For example, in some embodiments, in the compounds described herein hydrogen atoms may be replaced or substituted by one or more deuterium or tritium. Certain isotopically labeled compounds of this disclosure, for example, those incorporating a radioactive isotope, may be useful in drug or substrate tissue distribution studies. The radioactive isotopes tritium, i.e., ³H, and carbon 14, i.e., ¹⁴C, are particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. Suitable isotopes that may be incorporated in compounds described herein may include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. Substitution with positron emitting isotopes, such as ¹¹C, ¹⁸F, ¹⁵O, and ¹³N, may be useful in Positron Emission Topography (PET) studies.

The compounds of any of the formulae described herein may be prepared by methods known in the art of organic synthesis as set forth in part by the following synthetic schemes and examples in conjunction with the guidance provided herein. In the schemes described below, it is understood that protecting groups for sensitive or reactive groups may be employed where necessary in accordance with general principles or chemistry in accordance with the guidance provided herein. Protecting groups may be manipulated according to standard methods of organic synthesis (T. W. Greene and P. G. M. Wuts, “Protective Groups in Organic Synthesis,” Third edition, Wiley, New York 1999). These groups may be removed at a convenient stage of the compound synthesis using methods that are readily apparent to those skilled in the art based on the detailed teaching provided herein. The selection processes, as well as the reaction conditions and order of their execution, shall be consistent with the present disclosure.

The following Schemes 4-6 also illustrate the synthesis of a compound of formula (13) and its intermediates.

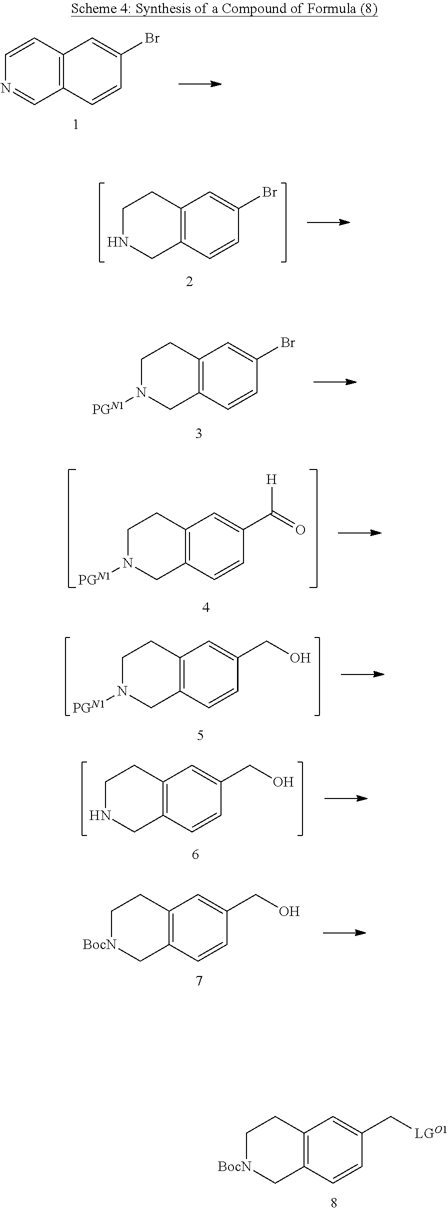

Scheme 4 shows the synthesis of a compound of formula (8), or a salt thereof.

### Synthesis of a Compound of Formula (2)

With continued reference to Scheme 4, in some embodiments, a compound of formula (1), a salt thereof,

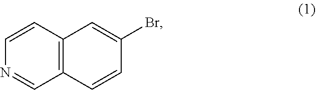 [SMILES: Brc1ccc2cnccc2c1]

may be contacted with a reducing agent, to form a compound of formula (2), or a salt thereof,

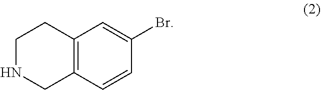 [SMILES: Brc1ccc2c(c1)CCNC2]

In some embodiments of the preparation of a compound of formula (2), or a salt thereof, the reducing reagent is LiBH₄, NaBH₄, lithium aluminum hydride (LAH), diisobutyl aluminum hydride (DIBAL), BH₃-dimethyl sulfide, or LiBEt₃H. In certain such embodiments, the reducing reagent is sodium borohydride (NaBH₄).

In some embodiments of the preparation of a compound of formula (2), or a salt thereof, the contacting of the compound of formula (1), or a salt thereof, with a reducing reagent may be performed in the presence of acetic acid. In certain such embodiments, the solvent is acetic acid.

In some embodiments of the preparation of a compound of formula (2), or a salt thereof, the contacting of the compound of formula (1), or a salt thereof, with a reducing reagent may be performed at a temperature of between about −10° C. to about 30° C. In some embodiments, the contacting of the compound of formula (1), or a salt thereof, with a reducing reagent may be performed at a temperature of between about 0° C. to about 30° C. In some embodiments, the contacting of the compound of formula (1), or a salt thereof, with a reducing reagent may be performed at a temperature of between about 10° C. to about 30° C. In some embodiments, the contacting of the compound of formula (1), or a salt thereof, with a reducing reagent may be performed at a temperature of between about 15° C. to about 25° C.

In some embodiments, the compound of formula (2), or a salt thereof, can be used in the next reaction without substantial purification. In some embodiments, the compound of formula (2), or a salt thereof, can be used in the next reaction as solution in a solvent. In certain such embodiments, the solvent is dichloromethane, tetrahydrofuran (THF), 2-Me-THF, dimethylformamide (DMF), acetonitrile, or a combination of any of the foregoing. In certain such embodiments, the solvent is dichloromethane.

### Synthesis of a Compound of Formula (3)

With continued reference to Scheme 4, a compound of formula (3):

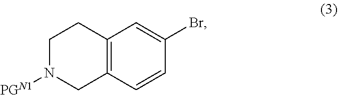

or a salt thereof, may be synthesized with contacting a compound of formula (2), or a salt thereof, with an amino protecting group reagent.

In some embodiments, the amino protecting group reagent is triphenylmethyl chloride, acetic anhydride, acetyl chloride, Fmoc-Cl, Teoc-Cl, Bpoc-N3, (Boc)₂O, Alloc-Cl, Cbz-Cl, PNZ-Cl, PMB-Cl, formic acetate anhydride, trihaloacetyl chloride (e.g., trifluoroacetyl chloride or trichloroacetyl chloride) trihaloacetic anhydride (e.g., trifluoroacetic anhydride or trichloroacetic anhydride), methyl chlorocarbonate, ethyl chlorocarbonate, benzoyl chloride, 2,3,4,5,6-pentafluorobenzoyl chloride, or phthalic anhydride. In some embodiments, the amino protecting group reagent is triphenylmethyl chloride.

In some embodiments, PG^{N1} is triphenylmethyl (trityl; Trt), Teoc, Bpoc, Boc, Alloc, Fmoc, Cbz, PNZ, formyl, acetyl, trihaloacetyl (e.g., trifluoroacetyl, trichloroacetyl), benzoyl, PMB, phthalimido, methoxycarbonyl, ethoxycarbonyl, or 2,3,4,5,6-pentafluorobenzoyl. In some embodiments, PG^{N1} is triphenylmethyl (trityl; Trt).

The contacting of the compound of formula (2), or a salt, with the amino protecting group reagent may be performed in the presence of an activating reagent. In some embodiments, the activating reagent is trimethylamine (TEA), 1,8-diazabicyclo[5.4.0]undec-7-ene (DBU), pyridine, piperidine, 4-dimethylaminopyridine (DMAP), 2,6-lutidine, dimethylaniline, N-methylpyrrilidone, N-diisopropylethylamine, N-methylimidazole, N-ethyldimethylamine, trimethylamine, or a combination of any of the foregoing. In some embodiments, the activating reagent is DMAP.

The contacting of the compound of formula (2), or a salt thereof, with the amino protecting group reagent may be performed in the presence of a solvent. In some embodiments, the solvent is dichloromethane (DCM), tetrahydrofuran (THF), 2-Me-THF, dimethylformamide (DMF), acetonitrile, or a combination of any of the foregoing. In some embodiments, the solvent is dichloromethane (DCM).

In some embodiments of the preparation of a compound of formula (3), or a salt thereof, the contacting of the compound of formula (2), or a salt thereof, with an amino protecting group reagent may be performed at a temperature of between about −10° C. to about 40° C. In some embodiments, the contacting of the compound of formula (2), or a salt thereof, with an amino protecting group reagent may be performed at a temperature of between about 0° C. to about 40° C. In some embodiments, the contacting of the compound of formula (2), or a salt thereof, with an amino protecting group reagent may be performed at a temperature of between about 10° C. to about 40° C. In some embodiments, the contacting of the compound of formula (2), or a salt thereof, with an amino protecting group reagent may be performed at a temperature of between about 20° C. to about 30° C.

In some embodiments, the compound of formula (3), or salt thereof, can be used in the next step without substantial purification. In some embodiments, the compound of formula (3), or salt thereof, is isolated.

### Synthesis of a Compound of Formula (4)

With continued reference to Scheme 4, a compound of formula (4):

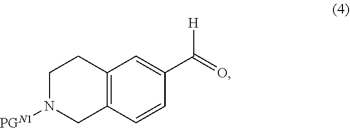

or a salt thereof, may be synthesized with contacting a compound of formula (3), or a salt thereof, with an organometallic reagent and dimethylformamide (DMF).

Bouveault aldehyde synthesis is referred to for preparation of a formyl group onto an alkyl or aryl group. Bouveault-aldehyde formation usually involves the reaction with magnesium, or a metal-halogen transfer agent, in an inert solvent, and the subsequent reaction with a formamide. In some embodiments, the organometallic reagent is an alkyl magnesium halide (e.g., Grignard reagent). In some embodiments, the alkyl magnesium halide is isopropyl magnesium chloride. In some embodiments, the organometallic reagent is an organolithium reagent.

The contacting of the compound of formula (3), or a salt thereof, with an organometallic reagent and dimethylformamide (DMF) may be performed in the presence of a solvent. In some embodiments, the solvent is dichloromethane (DCM), tetrahydrofuran (THF), 2-Me-THF, dimethylformamide (DMF), acetonitrile, or a combination of any of the foregoing. In some embodiments, the solvent is THIF.

In some embodiments, the compound of formula (4), or salt thereof, can be used in the next step without substantial purification. In some embodiments, the compound of formula (4), or a salt thereof, can be used in the next reaction as solution in a solvent. In certain such embodiments, the solvent is dichloromethane (DCM), tetrahydrofuran (THF), 2-Me-THF, dimethylformamide (DMF), acetonitrile, or a combination of any of the foregoing. In certain such embodiments, the solvent is THF.

### Synthesis of a Compound of Formula (5)

In some embodiments, with reference to Scheme 4, a compound of formula (5):

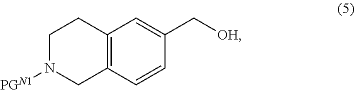

or a salt thereof, may be synthesized from contacting the compound of formula (3), or a salt thereof, with an organometallic/metal reagent and formaldehyde to yield a compound of formula (5), or a salt thereof.

With continued reference to Scheme 4, a compound of formula (5):

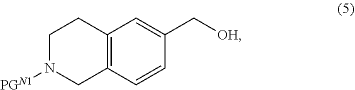

or a salt thereof, may be synthesized with contacting a compound of formula (4), or a salt thereof, with a reducing agent.

In some embodiments of the preparation of a compound of formula (5), or a salt thereof, the reducing reagent is LiBH₄, NaBH₄, lithium aluminum hydride (LAH), diisobutyl aluminum hydride (DIBAL), BH₃-dimethyl sulfide, or LiBEt₃H. In certain such embodiments, the reducing reagent is NaBH₄.

The contacting of the compound of formula (4), or a salt thereof, with the reducing reagent may be performed in the presence of a solvent. In some embodiments, the solvent is dichloromethane (DCM), tetrahydrofuran (THF), 2-Me-THF, dimethylformamide (DMF), acetonitrile, methanol, or a combination of any of the foregoing. In some embodiments, the solvent is THE and methanol.

In some embodiments, the compound of formula (5), or a salt thereof, can be used in the next reaction without substantial purification. In some embodiments, the compound of formula (5), or a salt thereof, can be used in the next reaction as solution in a solvent. In certain such embodiments, the solvent is methanol.

### Synthesis of a Compound of Formula (6)

With continued reference to Scheme 4, a compound of formula (6):

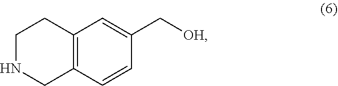 [SMILES: OCc1ccc2c(c1)CCNC2]

or a salt thereof, may be synthesized with contacting the compound of formula (5), or a salt thereof, with a PG^{N1} deprotecting reagent.

In some embodiments, the PG^{N1} deprotecting reagent is an acid. In some embodiments, the acid is HCl. In other embodiments, the PG^{N1} deprotecting reagent may be a reagent for hydrogenolysis.

The contacting of the compound of formula (5), or a salt thereof, with the PG^{N1}deprotecting reagent may be performed in the presence of a solvent. In some embodiments, the solvent is dichloromethane (DCM), tetrahydrofuran (THF), 2-Me-THF, dimethylformamide (DMF), acetonitrile, methanol, or a combination of any of the foregoing. In some embodiments, the solvent is dichloromethane and methanol.

In some embodiments of the preparation of a compound of formula (6), or a salt thereof, the contacting of the compound of formula (5), or a salt thereof, with a PG^{N1}deprotecting reagent may be performed at a temperature of between about −10° C. to about 30° C. In some embodiments, the contacting of the compound of formula (5), or a salt thereof, with a PG^{N1} deprotecting reagent may be performed at a temperature of between about 0° C. to about 30° C. In some embodiments, the contacting of the compound of formula (5), or a salt thereof, with a PG^{N1} deprotecting reagent may be performed at a temperature of between about 10° C. to about 25° C.

In some embodiments, the compound of formula (6), or a salt thereof, can be used in the next reaction without substantial purification. In some embodiments, the compound of formula (6), or a salt thereof, can be used in the next reaction as solution in a solvent. In certain such embodiments, the solvent is water.

### Synthesis of a Compound of Formula (7)

With continued reference to Scheme 4, a compound of formula (7):

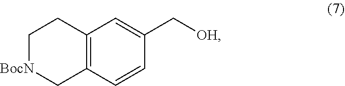 [SMILES: CC(C)(C)OC(=O)N1CCc2cc(CO)ccc2C1]

or a salt thereof, may be synthesized with contacting the compound of formula (6), or a salt thereof, with a Boc protecting group reagent.

In some embodiments, the Boc protecting group reagent is di-tert-butyl dicarbonate, N-(t-butoxycarbonyloxy)-5-norbornene-endo-2,3-dicarboximide, N-tert-butoxycarbonylimidazole, 2-(tert-butoxycarbonyloxyimino)-2-phenylacetonitrile, 2-(tert-butoxycarbonylthio)-4,6-dimethylpyrimidine, 1-tert-butoxycarbonyl-1,2,4-triazole, tert-butyl phenyl carbonate, N-(tert-butoxycarbonyloxy)phthalimide, or tert-butyl 2,4,5-trichlorophenyl carbonate. In some embodiments, the Boc protecting group reagent is Boc₂O (Boc anhydride; di-tert-butyl dicarbonate) or Boc-ONb (N-(t-butoxycarbonyloxy)-5-norbornene-endo-2,3-dicarboximide). In certain such embodiments, the Boc protecting group reagent is Boc₂O.

Further, the reaction between the compound of formula (6), or a salt thereof, and the Boc protecting group reagent may be performed in the presence of a base. In some embodiments, the base is K₂CO₃.

The contacting of the compound of formula (6), or a salt thereof, with Boc protecting group reagent may be performed in the presence of a solvent. In some embodiments, the solvent is dichloromethane (DCM), tetrahydrofuran (THF), 2-Me-THF, dimethylformamide (DMF), acetonitrile, methanol, or a combination of any of the foregoing. In some embodiments, the solvent is THF.

In some embodiments of the preparation of a compound of formula (7), or a salt thereof, the contacting of the compound of formula (6), or a salt thereof, with a Boc protecting group reagent may be performed at a temperature of between about −10° C. to about 30° C. In some embodiments, the contacting of the compound of formula (6), or a salt thereof, with a Boc protecting group reagent may be performed at a temperature of between about 0° C. to about 30° C. In some embodiments, the contacting of the compound of formula (6), or a salt thereof, with a Boc protecting group reagent may be performed at a temperature of between about 10° C. to about 25° C.

In some embodiments, the compound of formula (7), or salt thereof, can be used in the next step without substantial purification. In some embodiments, the compound of formula (7), or salt thereof, is isolated.

### Synthesis of a Compound of Formula (8)

With continued reference to Scheme 4, a compound of formula (8):

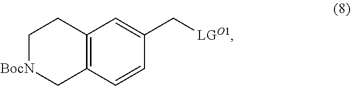

or a salt thereof, wherein −LG^{O1} is a leaving group, may be synthesized with contacting the compound of formula (7), or a salt thereof, with an alcohol activating reagent. An activating reagent refers to a reagent that converts the hydroxyl group into one that is more susceptible to nucleophilic attack. In some embodiments, the alcohol activating reagent is present in about 0.05 to 2.5 molar equivalents to the compound of formula (7). In some embodiments, the alcohol activating reagent is present in about 1.5 molar equivalents to the compound of formula (7).

In some embodiments, the alcohol activating reagent is a sulfonyl halide. Examples of sulfonyl halides include methanesulfonyl halide, (e.g., methanesulfonyl chloride; mesyl chloride; (CH₃SO₂Cl), toluenesulfonyl halide (e.g., toluenesulfonyl chloride; tosyl chloride; PhSO₂C), or nitrobenzenesulfonyl halide (e.g., 4-nitrobenzenesulfonyl chloride; nosyl chloride). In some embodiments, the alcohol activating reagent is methanesulfonyl chloride (mesyl chloride; CH₃SO₂Cl). In some embodiments, the alcohol activating reagent is a halogenating reagent that converts an alcohol to a halogen. Examples of these reagents include SO₂Cl, POCl, and PBr₃.

In some embodiments, −LG^{O1} is a sulfonate (e.g., mesylate, tosylate, or nosylate). In some embodiments, −LG^{O1} is mesylate (—O—SO₂CH₃), tosylate (—O—SO₂—C₆H₄—CH₃), or nosylate (—O—SO₂—C₆H₄—NO₂). In some embodiments, −LG^{O1} is a halogen, such as I, Br, or Cl.

The contacting of the compound of formula (7), or a salt thereof, with the alcohol activating reagent may be performed in the presence of a base. In some embodiments, the base is diisopropylethylamine (DIPEA), trimethylamine (TEA), N-ethyldimethylamine, or a combination of any of the foregoing. In some embodiments, the base is DIPEA.

The contacting of the compound of formula (7), or a salt thereof, with the the alcohol activating reagent may be performed in the presence of a solvent. In some embodiments, the solvent is dichloromethane (DCM), tetrahydrofuran (THF), 2-Me-THF, dimethylformamide (DMF), acetonitrile, or a combination of any of the foregoing. In some embodiments, the solvent is dichloromethane (DCM).

In some embodiments of the preparation of a compound of formula (8), or a salt thereof, the contacting of the compound of formula (7), or a salt thereof, with an alcohol activating reagent may be performed at a temperature of between about −20° C. to about 20° C. In some embodiments, the contacting of the compound of formula (7), or a salt thereof, with an alcohol activating reagent may be performed at a temperature of between about −10° C. to about 20° C. In some embodiments, the contacting of the compound of formula (7), or a salt thereof, with an alcohol activating reagent may be performed at a temperature of between about −10° C. to about 0° C.

In some embodiments, the compound of formula (8), or salt thereof, can be used in the next step without substantial purification. In some embodiments, the compound of formula (8), or salt thereof, is isolated.

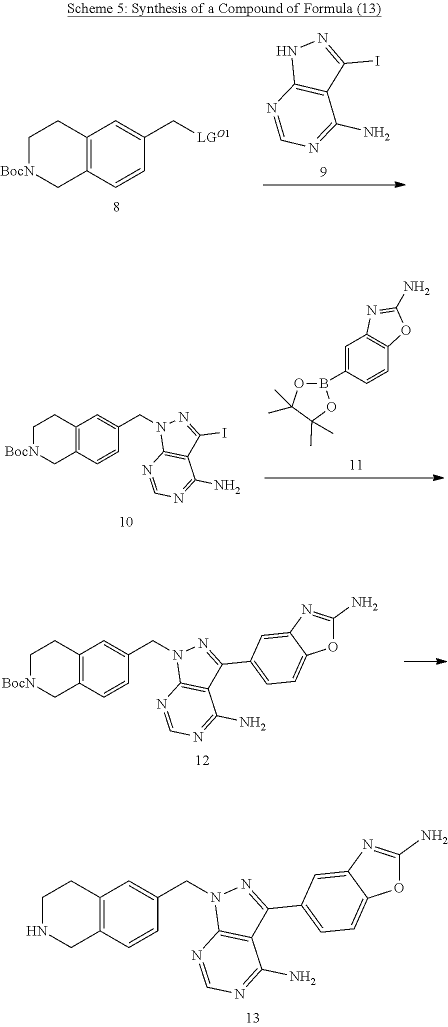

Scheme 5 shows the synthesis of a compound of formula (13), or a salt thereof.

### Synthesis of a Compound of Formula (10)

With continued reference to Scheme 5, a compound of formula (10):

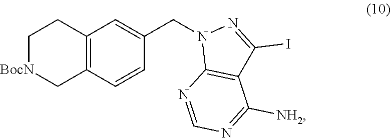 [SMILES: CC(C)(C)OC(=O)N1CCc2cc(Cn3nc(I)c4c(N)ncnc43)ccc2C1]

or a salt thereof, may be synthesized with contacting the compound of formula (8), or a salt thereof, with a compound of formula (9), or a salt thereof,

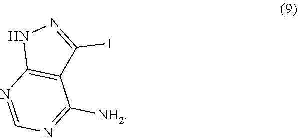 [SMILES: Nc1ncnc2[nH]nc(I)c12]

The reaction between the compound of formula (8), or a salt thereof, and the compound of formula (9), or a salt thereof, may be performed in the presence of a base. In some embodiments, the base is K₂CO₃.

The contacting of the compound of formula (8), or a salt thereof, with the compound of formula (9), or a salt thereof, may be performed in the presence of a solvent. In some embodiments, the solvent is dichloromethane (DCM), tetrahydrofuran (THF), 2-Me-THF, dimethylformamide (DMF), acetonitrile, methanol, or a combination of any of the foregoing. In some embodiments, the solvent is DMF.

In some embodiments of the preparation of a compound of formula (10), or a salt thereof, the contacting of the compound of formula (8), or a salt thereof, with the compound of formula (9), or a salt thereof, may be performed at a temperature of between about −10° C. to about 30° C. In some embodiments, the contacting of the compound of formula (8), or a salt thereof, with the compound of formula (9), or a salt thereof, may be performed at a temperature of between about 0° C. to about 30° C. In some embodiments, the contacting of the compound of formula (8), or a salt thereof, with the compound of formula (9), or a salt thereof, may be performed at a temperature of between about 10° C. to about 30° C. In some embodiments, the contacting of the compound of formula (8), or a salt thereof, with the compound of formula (9), or a salt thereof, may be performed at a temperature of between about 10° C. to about 25° C. In some embodiments, the contacting of the compound of formula (8), or a salt thereof, with the compound of formula (9), or a salt thereof, may be performed at a temperature of between about 20° C. to about 25° C.

In some embodiments, the compound of formula (10), or salt thereof, can be used in the next step without substantial purification. In some embodiments, the compound of formula (10), or salt thereof, is isolated.

### Synthesis of a Compound of Formula (12)

With continued reference to Scheme 5, a compound of formula (12):

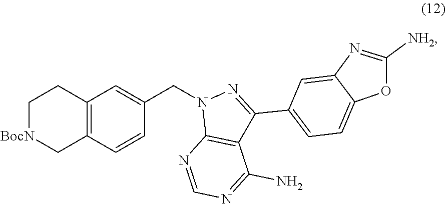 [SMILES: CC(C)(C)OC(=O)N1CCc2cc(Cn3nc(-c4ccc5oc(N)nc5c4)c4c(N)ncnc43)ccc2C1]

or a salt thereof, may be synthesized with contacting the compound of formula (10), or a salt thereof, with a compound of formula (11), or a salt thereof,

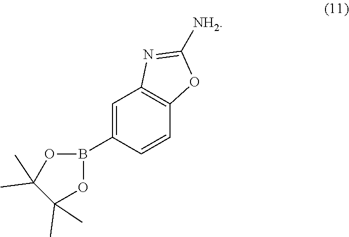 [SMILES: CC1(C)OB(c2ccc3oc(N)nc3c2)OC1(C)C]

The compound of formula (11) is described below in Synthesis of a Compound of Formula (11).

The Suzuki reaction can be referred to for coupling of aryl and heteroaryl groups. The Suzuki reaction is a cross-coupling reaction, where the coupling partners are a boronic acid and an organohalide and the catalyst is a palladium(0) complex.

The reaction between the compound of formula (10), or a salt thereof, and the compound of formula (11), or a salt thereof, may be performed in the presence of a palladium catalyst. In some embodiments, the palladium catalyst is Pd(PPh₃)₄.

The reaction between the compound of formula (10), or a salt thereof, and the compound of formula (11), or a salt thereof, may be performed in the presence of a base. In some embodiments, the base is K₂CO₃.

The contacting of the compound of formula (10), or a salt thereof, with the compound of formula (11), or a salt thereof, may be performed in the presence of a solvent. In some embodiments, the solvent is dioxane, water, tetrahydrofuran (THF), dimethylformamide (DMF), acetonitrile, methanol, or a combination of any of the foregoing. In some embodiments, the solvent is dioxane and water.

In some embodiments of the preparation of a compound of formula (12), or a salt thereof, the contacting of the compound of formula (10), or a salt thereof, with the compound of formula (11), or a salt thereof, may be performed at a temperature of between about 50° C. to about 100° C. In some embodiments, the contacting of the compound of formula (10), or a salt thereof, with the compound of formula (11), or a salt thereof, may be performed at a temperature of between about 70° C. to about 90° C. In some embodiments, the contacting of the compound of formula (10), or a salt thereof, with the compound of formula (11), or a salt thereof, may be performed at a temperature of between about 82° C. to about 87° C.

In some embodiments, the compound of formula (12), or salt thereof, can be used in the next step without substantial purification. In some embodiments, the compound of formula (12), or salt thereof, is isolated.

### Synthesis of a Compound of Formula (13)

With continued reference to Scheme 5, a compound of formula (13):

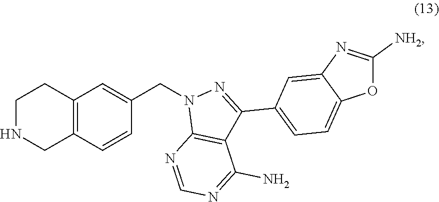 [SMILES: Nc1nc2cc(-c3nn(Cc4ccc5c(c4)CCNC5)c4ncnc(N)c34)ccc2o1]

or a salt thereof, may be synthesized with contacting the compound of formula (12), or a salt thereof, with a Boc removing agent.

In some embodiments, the Boc removing agent is an acid. In some embodiments, the acid is TFA, MsOH (methanesulfonic acid or CH₃SO₃H), PTSA (p-toluenesulfonic acid or tosylic acid), H₂SO₄, or HCl. In some embodiments, the acid is HCl or TFA. In some embodiments, acid is HCl. In some embodiments, acid is TFA.

The contacting of the compound of formula (13), or a salt thereof, with a Boc removing agent may be performed in the presence of a solvent. In some embodiments, the solvent is dioxane, water, tetrahydrofuran (THF), dimethylformamide (DMF), acetonitrile, methanol, or a combination of any of the foregoing. In some embodiments, the solvent is water.

In some embodiments of the preparation of a compound of formula (13), or a salt thereof, the contacting of the compound of formula (12), or a salt thereof, with a Boc removing agent may be performed at a temperature of between about −10° C. to about 30° C. In some embodiments, the contacting of the compound of formula (12), or a salt thereof, with a Boc removing agent may be performed at a temperature of between about 0° C. to about 30° C. In some embodiments, the contacting of the compound of formula (12), or a salt thereof, with a Boc removing agent may be performed at a temperature of between about 10° C. to about 30° C. In some embodiments, the contacting of the compound of formula (12), or a salt thereof, with a Boc removing agent may be performed at a temperature of between about 15° C. to about 25° C.

Salt formation with an acid may be performed to yield a salt of a compound of formula (13). If the compound of formula (13) is already a salt, the salt may be removed to afford a compound of formula (13) prior to formation of a different salt.

In some embodiments, the acid in the salt formation step is hydrochloric acid, thereby yielding an HCl salt of a compound of formula (13a),

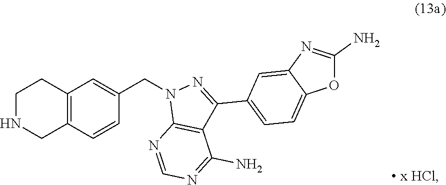

wherein x is 1, 2, or 3. In certain embodiments, x is 3, shown as a compound of formula (13b),

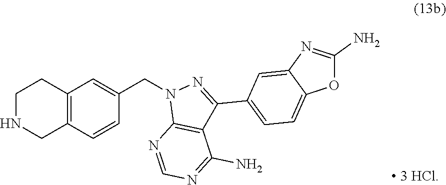 [SMILES: Nc1nc2cc(-c3nn(Cc4ccc5c(c4)CCNC5)c4ncnc(N)c34)ccc2o1]

In some embodiments, the acid in the salt formation step is trifluoroacetic acid, thereby yielding a TFA salt of a compound of formula (13c),

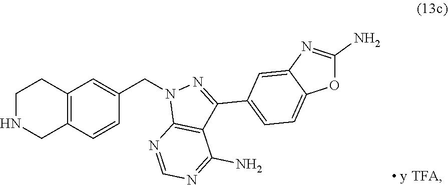

wherein y is 1, 2, or 3. In certain embodiments, y is 3, shown as a compound of formula (13d),

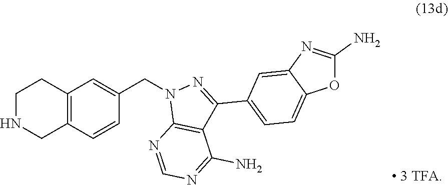 [SMILES: Nc1nc2cc(-c3nn(Cc4ccc5c(c4)CCNC5)c4ncnc(N)c34)ccc2o1]

The disclosure further provides for a process for preparing a crystalline form of compound of formula (13a), (13b), (13c), or (13d). Crystallization may aid in the purification process (e.g., lowering impurities) and simplifies purification compared to prior methods of purification. Crystallization may also act to purge of impurities. In certain embodiments, crystallization can be performed in alcohol, such as isopropanol.

In some embodiments, the compound of formula (13), or salt thereof, can be used in the next step without substantial purification. In some embodiments, the compound of formula (13), or salt thereof, is isolated. In some embodiments, the compound of formula (13a), (13b), (13c), or (13d) can be used in the next step without substantial purification. In some embodiments, the compound of formula (13a), (13b), (13c), or (13d) is isolated.

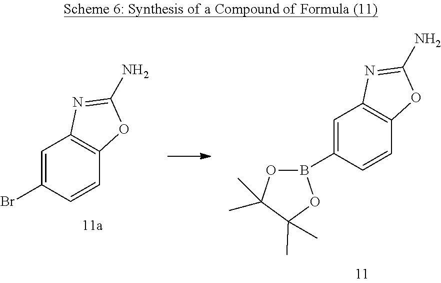 [SMILES: Nc1nc2cc(Br)ccc2o1 ; CC1(C)OB(c2ccc3oc(N)nc3c2)OC1(C)C]

Scheme 6 shows the synthesis of a compound of formula (11), or a salt thereof.

### Synthesis of a Compound of Formula (1)

With continued reference to Scheme 6, a compound of formula (11):

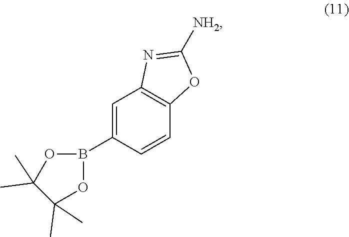 [SMILES: CC1(C)OB(c2ccc3oc(N)nc3c2)OC1(C)C]

or a salt thereof, is prepared by borylation of a compound of formula (11a), or a salt thereof,

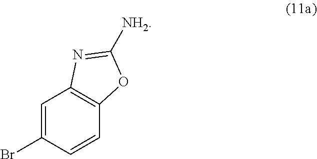 [SMILES: Nc1nc2cc(Br)ccc2o1]

Borylation reactions are transition metal catalyzed organic reactions that produce an organoboron compound through functionalization of aliphatic and aromatic C—H bonds and are useful reactions for carbon-hydrogen bond activation. The Miyaura borylation reaction enables the synthesis of boronates by cross-coupling of bis(pinacolato)diboron (B₂pin₂) with aryl halides and vinyl halides.

In some embodiments, the borylation is performed with contact with a boronic ester reagent. In some embodiments, the boronic ester reagent is bis(pinacolato)diboron (B₂Pin₂).

The reaction to prepare the compound of formula (11), or a salt thereof, may be performed in the presence of a palladium catalyst. In some embodiments, the palladium catalyst is Pd(dppf)Cl₂.

The reaction to prepare the compound of formula (11), or a salt thereof, may be performed in the presence of a solvent. In some embodiments, the solvent is toluene.

In some embodiments, the compound of formula (11), or salt thereof, can be used in the next step without substantial purification. In some embodiments, the compound of formula (11), or salt thereof, is isolated.

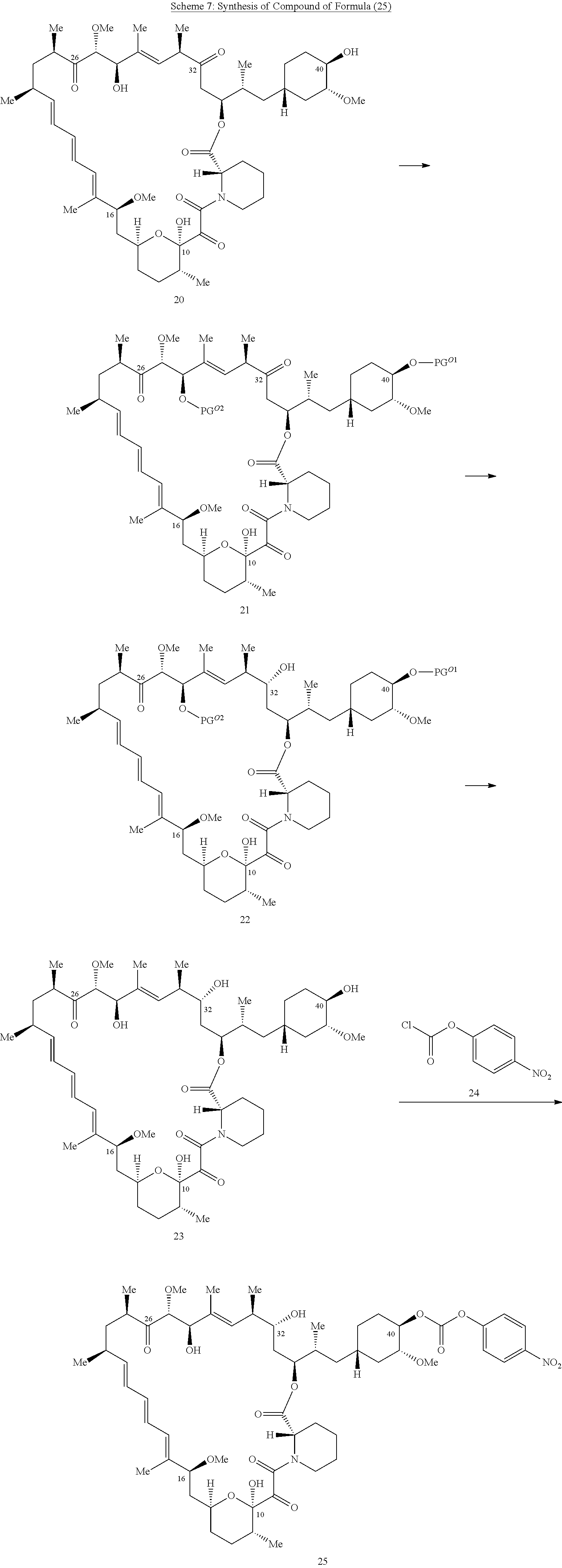

Scheme 7 shows the synthesis of a compound of formula (25), or a salt thereof.

### Synthesis of a Compound of Formula (21)

With continued reference to Scheme 7, a compound of formula (21):

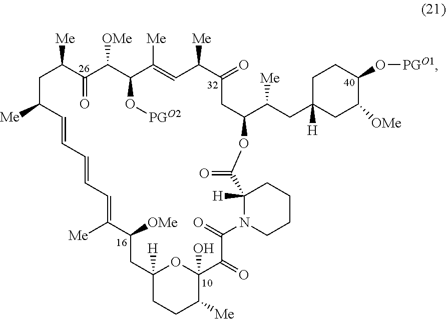

or a salt thereof, wherein PG^{O1} and PG^{O2} are the same or different at each instance a hydroxyl protecting group, may be synthesized with contacting a compound of formula (20), or a salt thereof, with a hydroxyl protecting group reagent.

In some embodiments, each hydroxyl protecting group reagent is a reagent used to put on a group selected from the group consisting of —C₁₋₆ alkyl, tri-C₁₋₆ alkylsilyl, —C₁₋₆ alkanoyl, benzoyl, benzyl, p-methoxybenzyl, 9-fluorenylmethyl, and diphenylmethyl as a hydroxyl protecting group. In some embodiments, the hydroxyl protecting group reagent is

In some embodiments, PG^{O1} and PG^{O2} are independently —C₁₋₆ alkyl, tri-C₁₋₆ alkylsilyl, —C₁₋₆ alkanoyl, benzoyl, benzyl, p-methoxybenzyl, 9-fluorenylmethyl, or diphenylmethyl. In some embodiments, PG^{O1} is triethylsilyl ether (TES). In some embodiments, PG^{O2} is triethylsilyl ether (TES).

The contacting of the compound of formula (20), or a salt, with the hydroxyl protecting group reagent may be performed in the presence of an activating reagent. In some embodiments, the activating reagent is imidazole.

The contacting of the compound of formula (20), or a salt thereof, with the hydroxyl protecting group reagent may be performed in the presence of a solvent. In some embodiments, the solvent is dichloromethane (DCM), tetrahydrofuran (THF), 2-Me-THF, dimethylformamide (DMF), acetonitrile, or a combination of any of the foregoing. In some embodiments, the solvent is dichloromethane (DCM).

In some embodiments of the preparation of a compound of formula (21), or a salt thereof, the contacting of the compound of formula (20), or a salt thereof, with a hydroxyl protecting group reagent may be performed at a temperature of between about −10° C. to about 30° C. In some embodiments, the contacting of the compound of formula (20), or a salt thereof, with a hydroxyl protecting group reagent may be performed at a temperature of between about −5° C. to about 20° C. In some embodiments, the contacting of the compound of formula (20), or a salt thereof, with a hydroxyl protecting group reagent may be performed at a temperature of between about −5° C. to about 10° C. In some embodiments, the contacting of the compound of formula (20), or a salt thereof, with a hydroxyl protecting group reagent may be performed at a temperature of between about −5° C. to about 5° C.

In some embodiments, the compound of formula (21), or salt thereof, undergoes a Florisil® filtration. In some embodiments, the Florisil® filtration is run with a solvent, such as dichloromethane (DCM). The compound of formula (21), or salt thereof, then undergoes an extraction with an aqueous solvent. In some embodiments, the aqueous solvent is aqueous NaCl/aqueous NaHCO₃. In some embodiments, the organic solvent is exchanged to another organic solvent, such as THF.

In some embodiments, the compound of formula (21), or salt thereof, can be used in the next step without substantial purification. In some embodiments, the compound of formula (21), or salt thereof, is isolated.

### Synthesis of a Compound of Formula (22)

With continued reference to Scheme 7, a compound of formula (22):

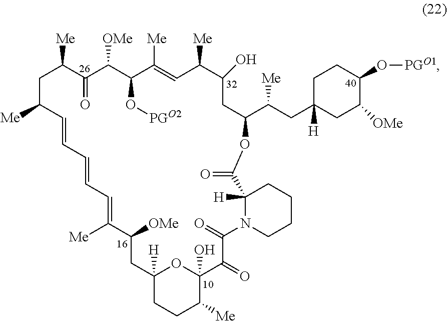

or a salt thereof, may be synthesized with contacting a compound of formula (21), or a salt thereof, with a reducing agent. In certain embodiments, the reaction includes subsequent contact with an oxidizing reagent to oxidize the groups that were undesirably reduced.

In some embodiments, the compound of formula (22), or salt thereof, has the following formula:

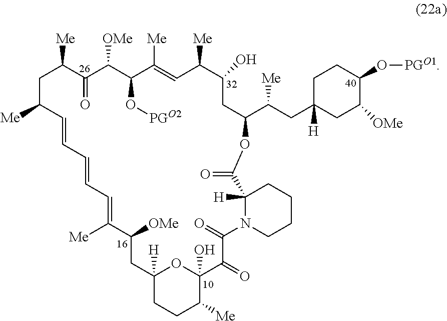

In some embodiments of the preparation of a compound of formula (22), or a salt thereof, the reducing reagent is LiAl(Ot-Bu)₃H, LiBH₄, NaBH₄, lithium aluminum hydride (LAH), diisobutyl aluminum hydride (DIBAL), BH₃-dimethyl sulfide, or LiBEt₃H. In certain such embodiments, the reducing reagent is LiAl(Ot-Bu)₃H.

The contacting of the compound of formula (21), or a salt thereof, with the reducing reagent may be performed in the presence of a solvent. In some embodiments, the solvent is dichloromethane, tetrahydrofuran (THF), 2-Me-THF, dimethylformamide (DMF), acetonitrile, methanol, or a combination of any of the foregoing. In some embodiments, the solvent is THIF.

In some embodiments, the compound of formula (21), or a salt thereof, can be used in the next reaction without substantial purification.

In some embodiments, the contacting of the compound of formula (21), or salt thereof, with the reducing agent is quenched with citric acid. In certain embodiments, after reaction of the compound of formula (21), or salt thereof, with the reducing agent, the reaction is diluted with an organic solvent, such as ethyl acetate, and then quenched with citric acid. In certain embodiments, after reaction of the compound of formula (21), or salt thereof, with the reducing agent, the reaction is quenched with citric acid and then diluted with an organic solvent, such as ethyl acetate. In some embodiments, the quenched compound is used in the next step after an exchange with another organic solvent. In some embodiments, the organic solvent is dichloromethane (DCM).

In certain embodiments, the reaction includes subsequent contact with an oxidizing reagent to oxidize the groups that were undesirably reduced. In certain embodiments, the product from the reduction is subsequently contacted with Cu(OAc)₂. The reaction with Cu(OAc)₂ may be performed in the presence of a solvent. In some embodiments, the solvent is dichloromethane (DCM), tetrahydrofuran (THF), 2-Me-THF, dimethylformamide (DMF), acetonitrile, or a combination of any of the foregoing. In some embodiments, the solvent is dichloromethane (DCM).

In some embodiments, the compound of formula (22), or salt thereof, undergoes a filtration. In some embodiments, the filtration is run with a solvent, such as dichloromethane (DCM). The compound of formula (22), or salt thereof, then undergoes an extraction with an aqueous solvent. In some embodiments, the aqueous solvent is aqueous NaCl/aqueous NaHCO₃.

In some embodiments, the compound of formula (22), or salt thereof, can be used in the next step without substantial purification. In some embodiments, the compound of formula (22), or salt thereof, is isolated. In some embodiments, the compound of formula (22), or salt thereof, can be used in the next step in an organic solvent, such as THF.

### Synthesis of a Compound of Formula (23)

With continued reference to Scheme 7, a compound of formula (23):

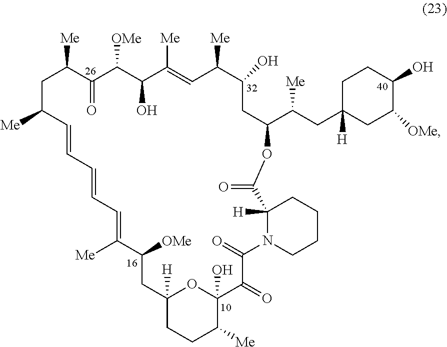 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a salt thereof, may be synthesized with contacting the compound of formula (22), or a salt thereof, with a PG^{O1} deprotecting reagent and a PG^{O2} deprotecting reagent. The removal of PG^{O1} and PG^{O2} may depend on the the identity of the protecting groups. In some embodiments, when PG^{O1} and PG^{O2} are silyl ethers, deprotecting may be accomplished with an acid or a fluoride (e.g., tetra-n-butylammonium fluoride; TBAF).

In some embodiments, the PG^{O1} deprotecting reagent is an acid. In some embodiments, the acid is HF.

In some embodiments, the PG^{O1} deprotecting reagent is an acid. In some embodiments, the acid is HF.

The contacting of the compound of formula (22), or a salt, with the PG^{O1}deprotecting reagent and a PG^{O2} deprotecting reagent may be performed in the presence of a base. In some embodiments, the base is pyridine.

The contacting of the compound of formula (22), or a salt thereof, with the PG^{O1}deprotecting reagent and PG^{O2} deprotecting reagent may be performed in the presence of a solvent. In some embodiments, the solvent is dichloromethane (DCM), tetrahydrofuran (THF), 2-Me-THF, dimethylformamide (DMF), acetonitrile, methanol, or a combination of any of the foregoing. In some embodiments, the solvent is THF.

In some embodiments, the compound of formula (23), or salt thereof, can be used in the next step without substantial purification. In some embodiments, the compound of formula (23), or salt thereof, is isolated.

### Synthesis of a Compound of Formula (25)

With continued reference to Scheme 7, a compound of formula (25):

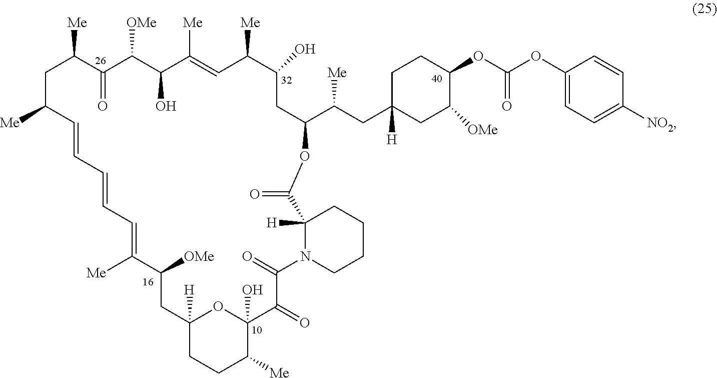 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

or a salt thereof, may be synthesized with contacting the compound of formula (23), or a salt thereof, with a compound of formula (24), or a salt thereof,

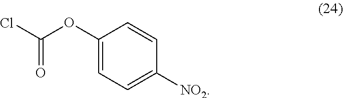 [SMILES: O=C(Cl)Oc1ccc([N+](=O)[O-])cc1]

The contacting of the compound of formula (23), or a salt thereof, with the compound of formula (24), or a salt thereof, may be performed in the presence of a solvent. In some embodiments, the solvent is dichloromethane (DCM), tetrahydrofuran (THF), 2-Me-THE, dimethylformamide (DMF), acetonitrile, or a combination of any of the foregoing. In some embodiments, the solvent is dichloromethane (DCM).

The contacting of the compound of formula (23), or a salt thereof, with the compound of formula (23), or a salt thereof, may be performed in the presence of a base. In some embodiments, the base is pyridine.

In some embodiments of the preparation of a compound of formula (25), or a salt thereof, the contacting of the compound of formula (23), or a salt thereof, with the compound of formula (24), or a salt thereof, may be performed at a temperature of between about −25° C. to about 20° C. In some embodiments, the contacting of the compound of formula (23), or a salt thereof, with the compound of formula (24), or a salt thereof, may be performed at a temperature of between about −25° C. to about 10° C. In some embodiments, the contacting of the compound of formula (23), or a salt thereof, with the compound of formula (24), or a salt thereof, may be performed at a temperature of between about −20° C. to about 5° C.

In some embodiments, the compound of formula (25), or salt thereof, can be used in the next step without substantial purification. In some embodiments, the compound of formula (25), or salt thereof, is isolated.

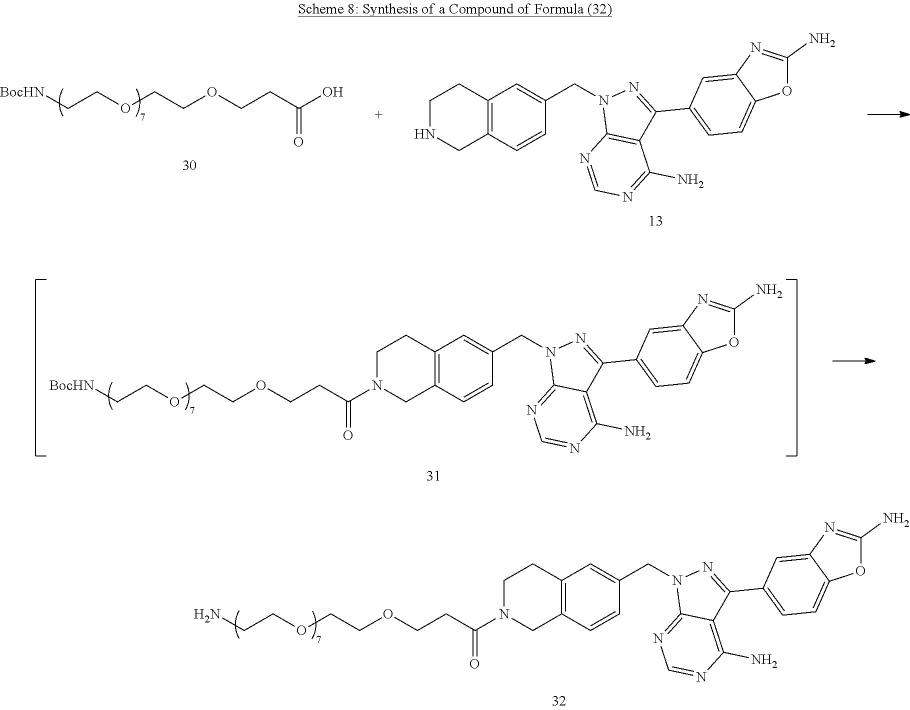

Scheme 8 shows the synthesis of a compound of formula (32).

### Synthesis of a Compound of Formula (31)

With continued reference to Scheme 8, a compound of formula (31):

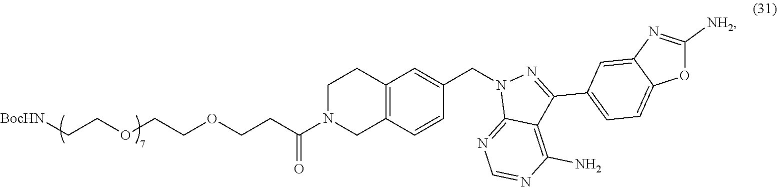

or a salt thereof, may be synthesized with contacting the compound of formula (13), or a salt thereof,

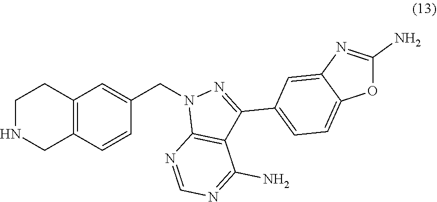 [SMILES: Nc1nc2cc(-c3nn(Cc4ccc5c(c4)CCNC5)c4ncnc(N)c34)ccc2o1]

with a compound of formula (30), or a salt thereof,

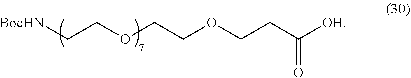

In some embodiments, one equivalent of the compound of formula (13) is used with one equivalent of the compound of formula (30).

In some embodiments, the compound of formula (13) is a compound of formula (13a):

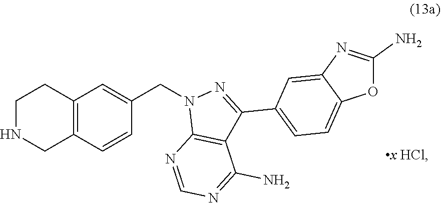

wherein x is 1, 2, or 3. In some embodiments, x is 3.

In some embodiments, the compound of formula (13) is a compound of formula (13c):

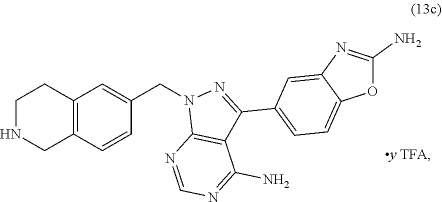

wherein y is 1, 2, or 3. In some embodiments, y is 3.

The reaction can be performed in the presence of an activating reagent. An activating reagent refers to a reagent that converts the carbonyl of a carboxylic acid group into one that is more susceptible to nucleophilic attack. In some embodiments, the activating reagent is HATU, HOOBt, HOSu, HOAt, DMAP, BOP, PyBOP, PyBrOP, PyAOP, PyOxim, DEPBT, TBTU, HBTU, HCTU, HDMC, COMU, CDI, or HOBt. In certain such embodiments, the activating reagent is HOBt.

The reaction can be performed in the presence of a coupling reagent. In some embodiments, the coupling reagent is DCC, EDCI, DIC, WSC, EDAC or PyBOP. In certain such embodiments, the coupling reagent is 1-ethyl-3-(3-dimethylaminopropyl)carbodiimide (EDCI).

The contacting of the compound of formula (30), or a salt thereof, with the compound of formula (13), or a salt thereof, may be performed in the presence of a solvent. In some embodiments, the solvent is dioxane, water, dichloromethane (DCM), dimethylacetamide (DMAc), tetrahydrofuran (THF), dimethylformamide (DMF), acetonitrile, methanol, or a combination of any of the foregoing. In some embodiments, the solvent is dimethylacetamide (DMAc).

In some embodiments of the preparation of a compound of formula (31), or a salt thereof, the contacting of the compound of formula (13), or a salt thereof, with the compound of formula (30), or a salt thereof, may be performed at a temperature of between about −10° C. to about 40° C. In some embodiments, the contacting of the compound of formula (13), or a salt thereof, with with the compound of formula (30), or a salt thereof, may be performed at a temperature of between about 0° C. to about 40° C. In some embodiments, the contacting of the compound of formula (13), or a salt thereof, with with the compound of formula (30), or a salt thereof, may be performed at a temperature of between about 10° C. to about 30° C. In some embodiments, the contacting of the compound of formula (13), or a salt thereof, with with the compound of formula (30), or a salt thereof, may be performed at a temperature of between about 15° C. to about 25° C.

In some embodiments, the compound of formula (31), or salt thereof, undergoes a filtration. In some embodiments, the filtration is run with a solvent, such as dichloromethane (DCM). The compound of formula (31), or salt thereof, then undergoes an extraction with an aqueous solvent. In some embodiments, the aqueous solvent is aqueous NaCl.

In some embodiments, the compound of formula (31), or salt thereof, can be used in the next step without substantial purification.

### Synthesis of a Compound of Formula (32)

With continued reference to Scheme 8, a compound of formula (32):

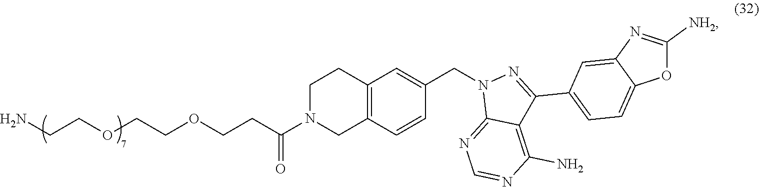

or a salt thereof, may be synthesized with contacting the compound of formula (31) or a salt thereof, with a Boc removing reagent. In some embodiments, one equivalent of the compound of formula (31) is used with one equivalent of the Boc removing reagent.

In some embodiments, the Boc removing agent is an acid. Examples of Boc removing reagents include, but are not limited to, TFA, aqueous phosphoric acid, methanesulfonic acid (MSA), SnCl₄, HCl, HCl/dioxane, and HCl/MeOH.

In some embodiments, the acid is TFA, MsOH (methanesulfonic acid or CH₃SO₃H), PTSA (p-toluenesulfonic acid or tosylic acid), H₂SO₄, or HCl. In some embodiments, the acid is TFA or MsOH. In some embodiments, acid is TFA, H₂SO₄, or HCl. In some embodiments, acid is HCl.

The contacting of the compound of formula (13), or a salt thereof, with a Boc removing agent may be performed in the presence of a solvent. In some embodiments, the solvent is dioxane, water, dichloromethane, dimethylacetamide (DMAc), tetrahydrofuran (THF), dimethylformamide (DMF), acetonitrile, methanol, or a combination of any of the foregoing. In some embodiments, the solvent is selected from the group consisting of water, dichloromethane, dimethylacetamide (DMAc), and mixtures thereof.

In some embodiments of the preparation of a compound of formula (13), or a salt thereof, the contacting of the compound of formula (12), or a salt thereof, with a Boc removing agent may be performed at a temperature of between about −10° C. to about 30° C. In some embodiments, the contacting of the compound of formula (12), or a salt thereof, with a Boc removing agent may be performed at a temperature of between about 0° C. to about 30° C. In some embodiments, the contacting of the compound of formula (12), or a salt thereof, with a Boc removing agent may be performed at a temperature of between about 10° C. to about 30° C. In some embodiments, the contacting of the compound of formula (12), or a salt thereof, with a Boc removing agent may be performed at a temperature of between about 15° C. to about 25° C.

The compound of formula (32), or salt thereof, undergoes a basification work-up with an aqueous solvent, such as aqueous NaOH and dichloromethane (DCM). The compound of formula (32), or salt thereof, then undergoes an extraction with an aqueous solvent, such as aqueous NaCl.

In some embodiments, the compound of formula (32), or salt thereof, can be used in the next step without substantial purification. In some embodiments, the compound of formula (32), or salt thereof, is isolated.

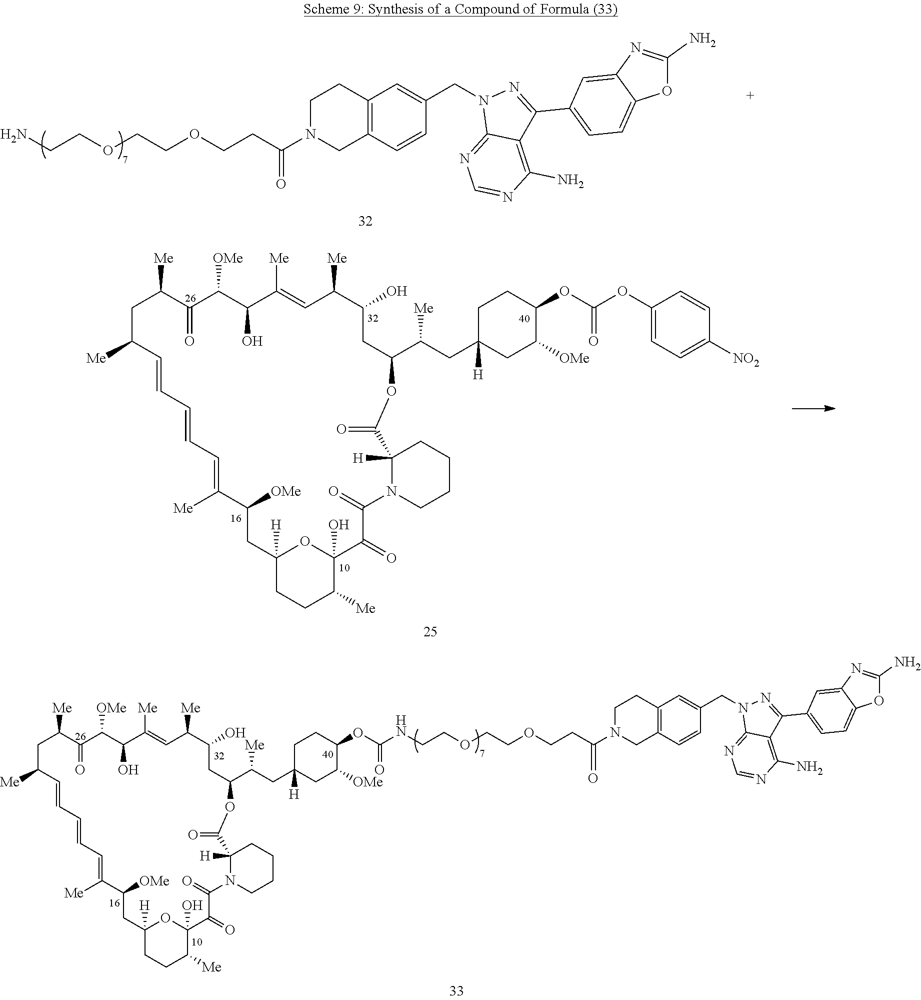

Scheme 9 shows the synthesis of a compound of formula (33), or a salt thereof.

### Synthesis of a Compound of Formula (33)

With continued reference to Scheme 9, a compound of formula (33):

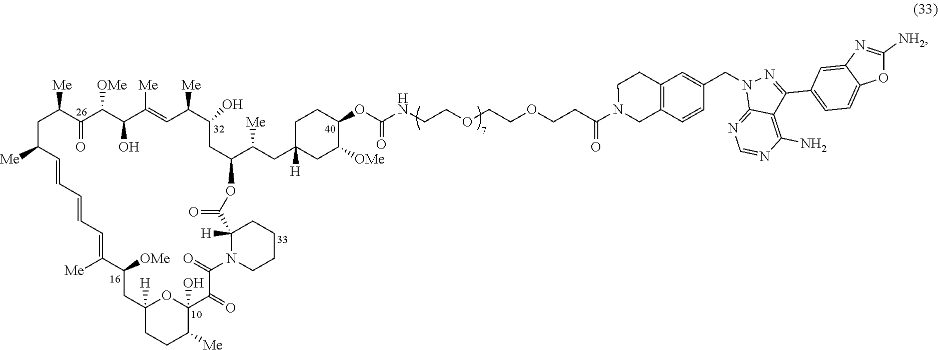

or a salt thereof, may be synthesized with contacting the compound of formula (32), or a salt thereof,

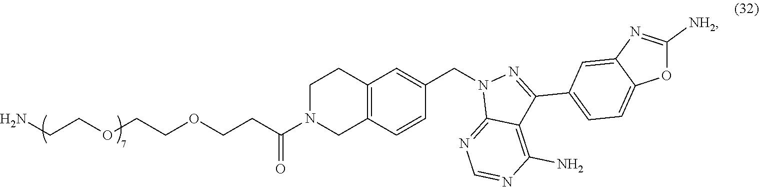

with a compound of formula (25), or a salt thereof,

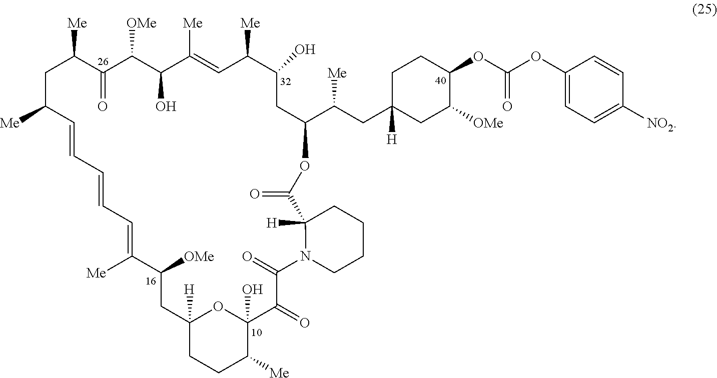 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

In some embodiments, one equivalent of the compound of formula (32) is used with one equivalent of the compound of formula (25).

The contacting of the compound of formula (32), or a salt thereof, with the compound of formula (25), or a salt thereof, may be performed in the presence of a solvent. In some embodiments, the solvent is dioxane, water, dichloromethane (DCM), dimethylacetamide (DMAc), tetrahydrofuran (THF), dimethylformamide (DMF), acetonitrile, methanol, or a combination of any of the foregoing. In some embodiments, the solvent is dimethylacetamide (DMAc).

In some embodiments of the preparation of a compound of formula (33), or a salt thereof, the contacting of the compound of formula (32), or a salt thereof, with the compound of formula (25), or a salt thereof, may be performed at a temperature of between about −20° C. to about 20° C. In some embodiments, the contacting of the compound of formula (32), or a salt thereof, with the compound of formula (25), or a salt thereof, may be performed at a temperature of between about −10° C. to about 20° C. In some embodiments, the contacting of the compound of formula (32), or a salt thereof, with the compound of formula (25), or a salt thereof, may be performed at a temperature of between about −10° C. to about 10° C.

In some embodiments, the compound of formula (33), or salt thereof, is isolated.

### Intermediate Compounds

In an aspect, the disclosure relates to intermediates in a synthetic process that may be used to synthesize the compound of formula (33), or a salt thereof.

The present disclosure provides a compound of formula (13), or a salt thereof:

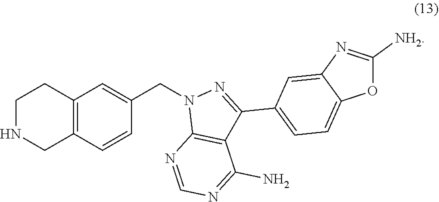 [SMILES: Nc1nc2cc(-c3nn(Cc4ccc5c(c4)CCNC5)c4ncnc(N)c34)ccc2o1]

The present disclosure provides a compound of formula (13a):

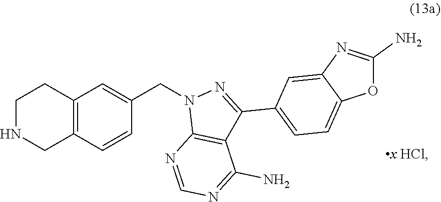

wherein n is 1, 2, or 3. In certain embodiments, x is 3:

The present disclosure provides a compound of formula (13b):

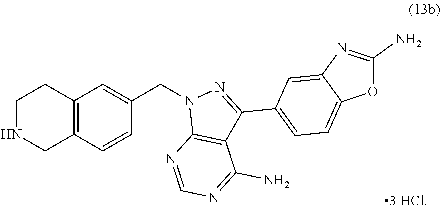 [SMILES: Nc1nc2cc(-c3nn(Cc4ccc5c(c4)CCNC5)c4ncnc(N)c34)ccc2o1]

The present disclosure provides a compound of formula (13c):

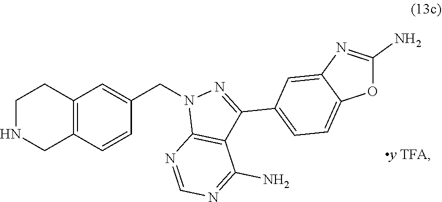

wherein y is 1, 2, or 3. In certain embodiments, y is 3:

The present disclosure provides a compound of formula (13d):

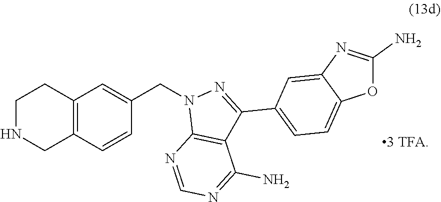 [SMILES: Nc1nc2cc(-c3nn(Cc4ccc5c(c4)CCNC5)c4ncnc(N)c34)ccc2o1]

The present disclosure provides a compound of formula (32), or a salt thereof:

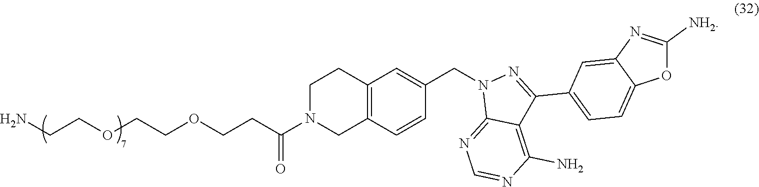

### Enumerated Embodiments

Some embodiments of this disclosure are Embodiment I, as follows:

Embodiment I-1. A process for preparing a compound of formula (3), or a salt thereof, comprising:

(1a) contacting a compound of formula (1), or a salt thereof,

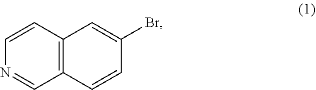 [SMILES: Brc1ccc2cnccc2c1]

with a reducing agent, to yield a compound of formula (2), or a salt thereof,

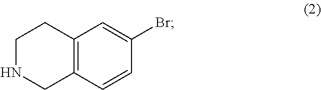 [SMILES: Brc1ccc2c(c1)CCNC2]

(2a) contacting a compound of formula (2), or a salt thereof, with an amino protecting group reagent to yield a compound of formula (3), or a salt thereof,

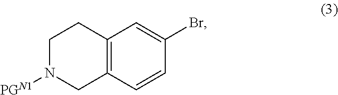

wherein PG^{N1} is an amino protecting group.

Embodiment I-2. The process of Embodiment I-1, wherein the reducing agent is sodium borohydride.

Embodiment I-3. The process of Embodiment I-1 or I-2, wherein step (1a) is performed in the presence of acetic acid.

Embodiment I-4. The process of any one of Embodiments I-1 to I-3, wherein the amino protecting group reagent is triphenylmethyl chloride.

Embodiment I-5. The process of any one of Embodiments I-1 to I-4, wherein PG^{N}1 is triphenylmethyl (trityl).

Embodiment I-6. The process of any one of Embodiments I-1 to I-5, wherein step (2a) is performed in the presence of an activating reagent.

Embodiment I-7. The process of Embodiment I-6, wherein the activating reagent is 4-dimethylaminopyridine (DMAP).

Embodiment I-8. The process of any one of Embodiments I-1 to I-7, wherein step (2a) is performed in dichloromethane (DCM).

Embodiment I-9. The process of any one of Embodiments I-1 to I-8, further comprising isolating the compound of formula (3).

Embodiment I-10. The process of any one of Embodiments I-1 to I-10, further comprising (3a′) contacting the compound of formula (3), or a salt thereof, with an organometallic/metal reagent and formaldehyde to yield a compound of formula (5), or a salt thereof,

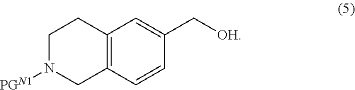

Embodiment I-11. The process of any one of Embodiment I-1 to I-10, further comprising

(3a) contacting the compound of formula (3), or a salt thereof, with an organometallic reagent and dimethylformamide (DMF) to yield a compound of formula (4), or a salt thereof,

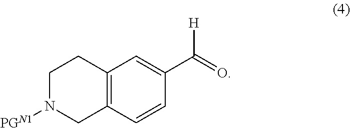

Embodiment I-12. The process of Embodiment I-10 or I-11, wherein the organometallic reagent is an alkyl magnesium halide.

Embodiment I-13. The process of any one of Embodiments I-10 to I-12, wherein step (3a) is performed in tetrahydrofuran (THF).

Embodiment I-14. The process of any one of Embodiment I-11 to I-13, further comprising

(4a) contacting the compound of formula (4), or a salt thereof, with a reducing agent to yield a compound of formula (5), or a salt thereof,

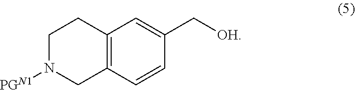

Embodiment I-15. The process of Embodiment I-10 or I-14, wherein the reducing agent is sodium borohydride.

Embodiment I-16. The process of any one of Embodiments I-10 and I-14 to I-15, wherein step (4a) is performed in a solvent selected from the group consisting of methanol, THF, and mixture thereof.

Embodiment I-17. The process of any one of Embodiments I-10 to I-16, further comprising

(5a) contacting the compound of formula (5), or a salt thereof, with a PG^{N}1 deprotecting reagent to yield a compound of formula (6), or a salt thereof,

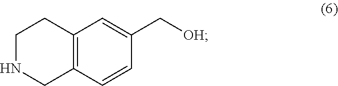 [SMILES: OCc1ccc2c(c1)CCNC2]

(6a) contacting the compound of formula (6), or a salt thereof, with a Boc protecting group reagent to yield a compound of formula (7), or a salt thereof,

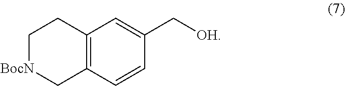 [SMILES: CC(C)(C)OC(=O)N1CCc2cc(CO)ccc2C1]

Embodiment I-18. The process of Embodiment I-17, wherein the PG^{N}1 deprotecting reagent is an acid.

Embodiment I-19. The process of Embodiment I-17 or I-18, wherein step (5a) is performed in DCM.

Embodiment I-20. The process of any one of Embodiments I-17 to I-19, wherein the Boc protecting group reagent is Boc₂O.

Embodiment I-21. The process of Embodiment I-20, wherein step (6a) is performed in THF.

Embodiment I-22. The process of any one of Embodiments I-17 to I-21, further comprising isolating the compound of formula (7).

Embodiment I-23. The process of any one of Embodiments I-17 to I-22, further comprising

(7a) contacting the compound of formula (7), or a salt thereof, with an alcohol activating reagent to yield a compound of formula (8), or a salt thereof,

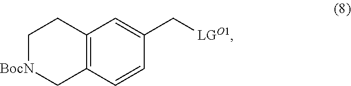

wherein −LG^{O1} is a leaving group.

Embodiment I-24. The process of Embodiment I-23, wherein the alcohol activating reagent is a sulfonyl halide or a halogenating reagent.

Embodiment I-25. The process of Embodiment I-23 or I-24, wherein the alcohol activating reagent is methanesulfonyl chloride (mesyl chloride; CH₃SO₂Cl).

Embodiment I-26. The process of Embodiment I-23, wherein −LG^{O1} is a sulfonate ester or a halide.

Embodiment I-27. The process of Embodiment I-23, wherein −LG^{O1} is mesylate (—O—SO₂CH₃).

Embodiment I-28. The process of any one of Embodiments I-23 to I-27, wherein step (7a) is performed in the presence of a base.

Embodiment I-29. The process of Embodiment I-28, wherein the base is diisopropylethylamine (DIPEA).

Embodiment I-30. The process of any one of Embodiments I-23 to I-29, wherein step (7a) is performed in DCM.

Embodiment I-31. The process of any one of Embodiment I-23 to I-30, further comprising isolating the compound of formula (8).

Embodiment I-32. The process of any one of Embodiments I-23 to I-31, further comprising

(8a) contacting the compound of formula (8), or a salt thereof, with a compound of formula (9), or a salt thereof,

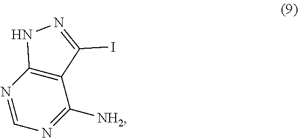 [SMILES: Nc1ncnc2[nH]nc(I)c12]

to yield a compound of formula (10), or a salt thereof,

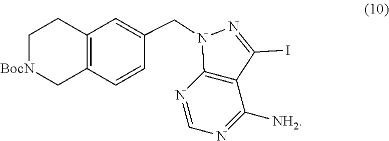 [SMILES: CC(C)(C)OC(=O)N1CCc2cc(Cn3nc(I)c4c(N)ncnc43)ccc2C1]

Embodiment I-33. The process of Embodiment I-32, wherein step (8a) is performed in DMF.

Embodiment I-34. The process of Embodiment I-32 or I-33, further comprising isolating the compound of formula (10).

Embodiment I-35. The process of any one of Embodiments I-32 to I-34, further comprising

(9a) contacting the compound of formula (10), or a salt thereof, with a compound of formula (11) or a salt thereof,

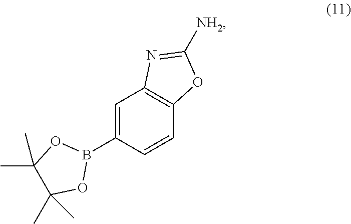 [SMILES: CC1(C)OB(c2ccc3oc(N)nc3c2)OC1(C)C]

to yield a compound of formula (12), or a salt thereof,

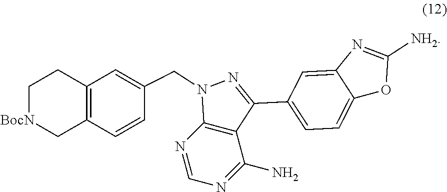 [SMILES: CC(C)(C)OC(=O)N1CCc2cc(Cn3nc(-c4ccc5oc(N)nc5c4)c4c(N)ncnc43)ccc2C1]

Embodiment I-36. The process of Embodiment I-35, wherein the compound of formula (11) is prepared by borylation of a compound of formula (11a), or a salt thereof,

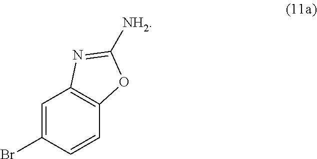 [SMILES: Nc1nc2cc(Br)ccc2o1]

Embodiment I-37. The process of Embodiment I-36, wherein borylation is performed with contact with a boronic ester reagent.

Embodiment I-38. The process of Embodiment I-37, wherein the boronic ester reagent is bis(pinacolato)diboron (B₂Pin₂).

Embodiment I-39. The process of any one of Embodiments I-35 to I-38, wherein step (9a) is performed in the presence of a palladium catalyst.

Embodiment I-40. The process of Embodiment I-39, wherein the palladium catalyst is Pd(PPh₃)₄.

Embodiment I-41. The process of any one of Embodiments I-35 to I-40, wherein step (9a) is performed in a solvent selected from the group consisting of water, dioxane, and mixture thereof.

Embodiment I-42. The process of any one of Embodiment I-35 to I-41, further comprising isolating the compound of formula (12).

Embodiment I-43. The process of any one of Embodiment I-35 to I-42, further comprising

(10a) contacting the compound of formula (12) with an acid to yield a compound of formula (13),

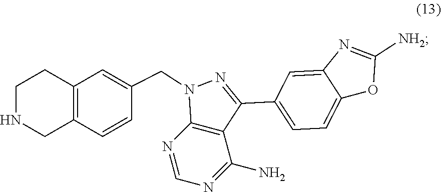 [SMILES: Nc1nc2cc(-c3nn(Cc4ccc5c(c4)CCNC5)c4ncnc(N)c34)ccc2o1]

(11a) preparing a salt of a compound of formula (13).

Embodiment I-44. The process of Embodiment I-43, wherein the acid is hydrochloric acid, thereby yielding a hydrochloric salt of compound of formula (13a),

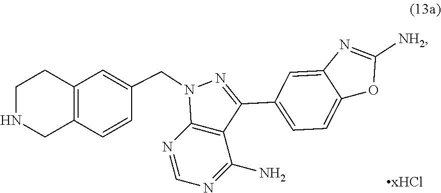

wherein x is 1, 2, or 3.

Embodiment I-45. The process of Embodiment I-44, wherein x is 3.

Embodiment I-46 The process of Embodiment I-43, wherein the acid is trifluoroacetic acid, thereby yielding a TFA salt of compound of formula (13c),

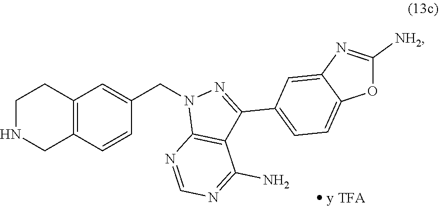

wherein y is 1, 2, or 3.

Embodiment I-47. The process of Embodiment I-46, wherein y is 3.

Embodiment I-48. The process of any one of Embodiments I-43 to I-47, wherein step (10a) and step (11a) are performed in water.

Embodiment I-49. The process of any one of Embodiment I-43 to I-48, further comprising isolating the compound of formula (13), (13a), or (13c).

Embodiment I-50. A process for preparing a compound of formula (21), or a salt thereof, comprising:

(1b) contacting a compound of formula (20), or a salt thereof,

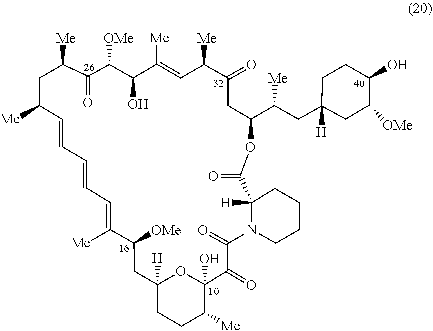 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

with a hydroxyl protecting group reagent, to yield a compound of formula (21), or a salt thereof,

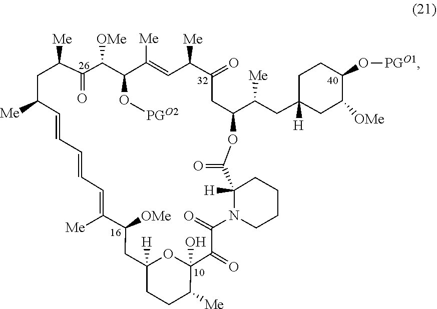

Embodiment I-52. The process of Embodiment I-50, wherein PG^{O2} is triethylsilyl ether (TES).

Embodiment I-53. The process of any one of Embodiments I-50 to I-53, wherein ether (TES).

Embodiment I-54. The process of any one of Embodiments I-50 to I-54, wherein step (1b) is performed in the presence of imidazole.

Embodiment I-55. The process of any one of Embodiments I-50 to I-54, wherein step (1b) is performed in DCM.

Embodiment I-56. The process of any one of Embodiments I-50 to I-54, wherein comprising isolating the compound of formula (21).

Embodiment I-57. The process of any one of Embodiment I-0 to I-5, further comprising:

(2b) contacting a compound of formula (21), or a salt thereof, with a reducing agent to yield a compound of formula (22), or a salt thereof,

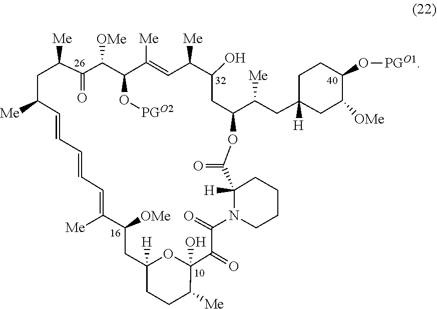

Embodiment I-57a. The process of any one of Embodiment I-50 to I-56, further comprising:

(2b) contacting a compound of formula (21), or a salt thereof, with a reducing agent to yield a compound of formula (22a), or a salt thereof,

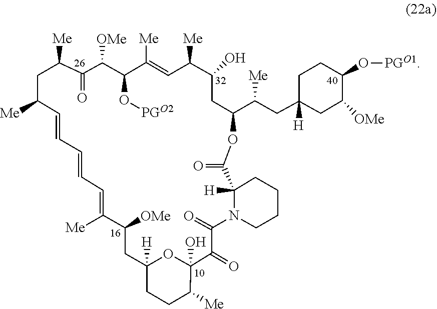

Embodiment I-58. The process of Embodiment I-57, wherein the reducing agent is LiAl(Ot-Bu)₃H.

Embodiment I-59. The process of Embodiment I-57 or I-58, wherein the product from step (2b) is subsequently contacted with Cu(OAc)₂.

Embodiment I-60. The process of any one of Embodiment I-57 to I-59, wherein step (2b) is performed in THF.

Embodiment I-61. The process of any one of Embodiment I-57 to I-60, further comprising isolating the compound of formula (22).

Embodiment I-62. The process of any one of Embodiment I-57 to I-61, further comprising:

(3b) contacting a compound of formula (22), or a salt thereof, with a PG^{O1}deprotecting reagent and a PG^{O2} deprotecting reagent to yield a compound of formula (23), or a salt thereof,

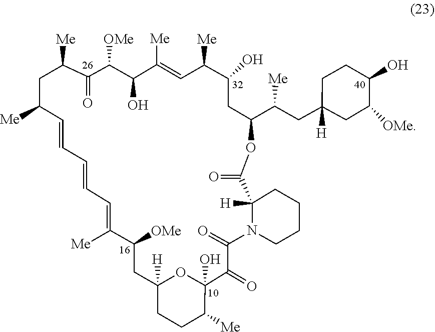 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Embodiment I-63. The process of Embodiment I-62, wherein the PG^{O1} deprotecting reagent is an acid.

Embodiment I-64. The process of Embodiment I-62, wherein the PG^{O2} deprotecting reagent is an acid.

Embodiment I-65. The process of any one of Embodiment I-62 to I-64, wherein step (3b) is performed in THF.

Embodiment I-66. The process of any one of Embodiment I-62 to I-65, further comprising isolating the compound of formula (23).

Embodiment I-67. The process of any one of Embodiment I-62 to I-66, further comprising (4b) contacting the compound of formula (23), or a salt thereof, with a compound of formula (24), or a salt thereof,

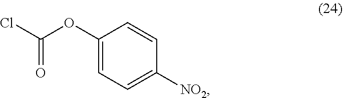 [SMILES: O=C(Cl)Oc1ccc([N+](=O)[O-])cc1]

to yield a compound of formula (25), or a salt thereof,

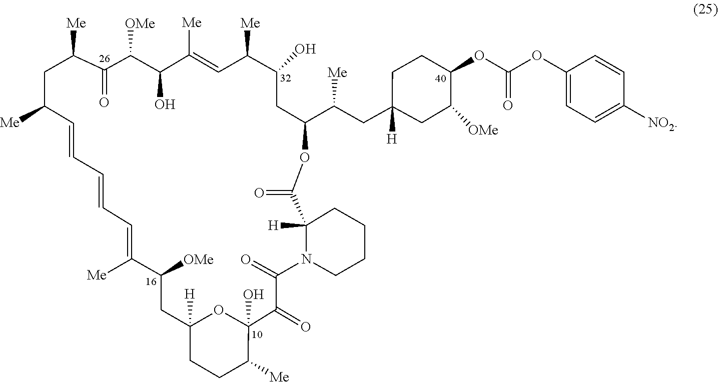 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Embodiment I-68. The process of Embodiment I-67, wherein step (4b) is performed in DCM.

Embodiment I-69. The process of Embodiment I-67 or I-68, further comprising isolating the compound of formula (25).

Embodiment I-70. A process for preparing a compound of formula (31), or a salt thereof, comprising:

(1c) contacting a compound of formula (30), or a salt thereof,

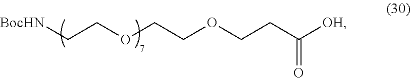

with a compound of formula (13), or a salt thereof,

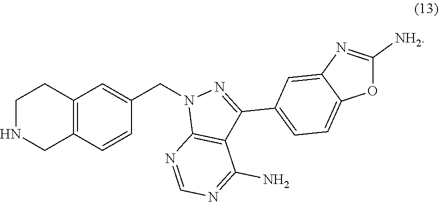 [SMILES: Nc1nc2cc(-c3nn(Cc4ccc5c(c4)CCNC5)c4ncnc(N)c34)ccc2o1]

to yield the compound of formula (31), or a salt thereof,

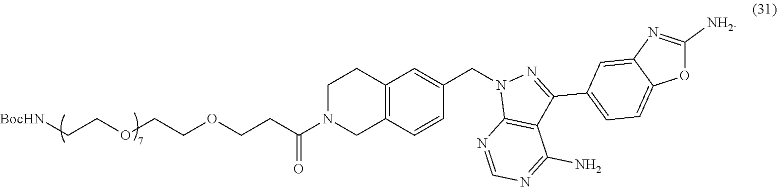

Embodiment I-71. The process of Embodiment I-70, wherein the compound of formula (13), or a salt thereof, is a compound of formula (13a),

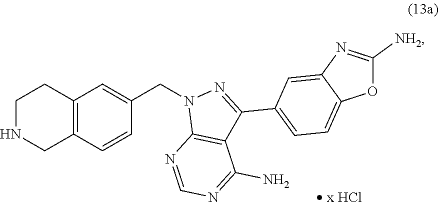

wherein n is 1, 2, or 3.

Embodiment I-72. The process of Embodiment I-71, wherein n is 3.

Embodiment I-73. The process of Embodiment I-70, wherein the compound of formula (13), or a salt thereof, is a compound of formula (13c),

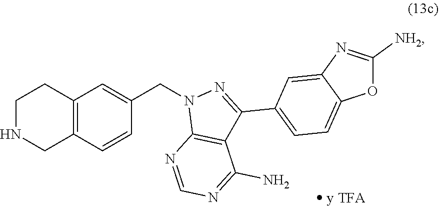

wherein y is 1, 2, or 3.

Embodiment I-74. The process of Embodiment I-73, wherein y is 3.

Embodiment I-75. The process of Embodiment I-70, wherein step (1c) is performed in the presence of a coupling reagent.

Embodiment I-76. The process of Embodiment I-75, wherein the coupling reagent is 1-ethyl-3-(3-dimethylaminopropyl)carbodiimide (EDCI).

Embodiment I-77. The process of any one of Embodiment I-70 to I-76, wherein step (1c) is performed in the presence of an activating reagent.

Embodiment I-78. The process of Embodiment I-77, wherein the activating reagent is hydroxybenzotriazole (HOBt).

Embodiment I-79. The process of any one of Embodiment I-70 to I-78, wherein step (1c) is performed in dimethylacetamide (DMM).

Embodiment I-80. The process of any one of Embodiment I-70 to I-79, further comprising isolating the compound of formula (31).

Embodiment I-81. The process of any one of Embodiment I-70 to I-80, further comprising

(2c) contacting the compound of formula (31) with a Boc removing agent to yield a compound of formula (32), or a salt thereof,

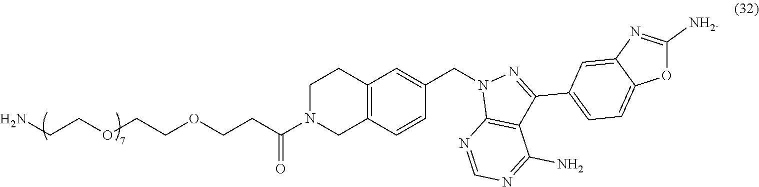

Embodiment I-82. The process of Embodiment I-81, wherein the Boc removing reagent is hydrochloric acid.

Embodiment I-83. The process of Embodiment I-81 or I-82, wherein step (2c) is performed in a solvent selected from the group consisting of water, DCM, dimethylacetamide (DMAc), and mixtures thereof.

Embodiment I-84. The process of any one of Embodiment I-81 to I-83, further comprising isolating the compound of formula (32).

Embodiment I-85. The process of any one of Embodiment I-81 to I-84, further comprising

(3c) contacting the compound of formula (32), or a salt thereof, with a compound of formula (25), or a salt thereof,

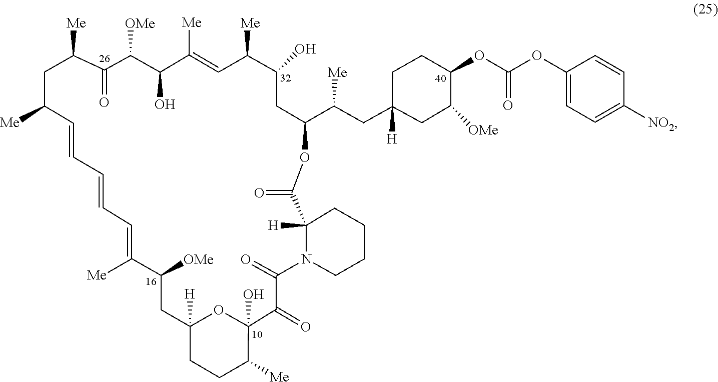 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

to yield a compound of formula (33), or a salt thereof,

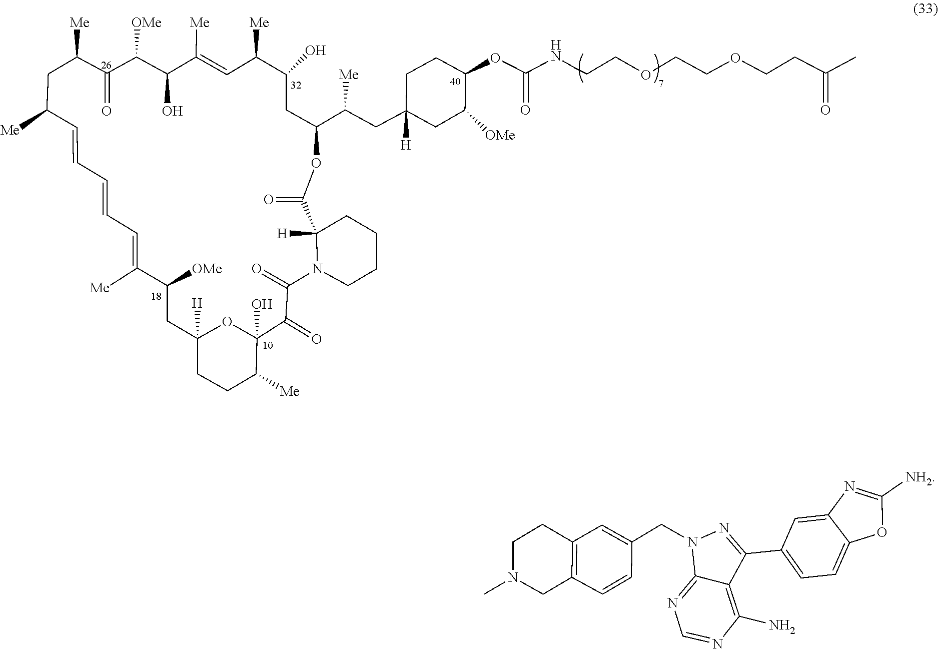

Embodiment I-86. The process of Embodiment I-85, wherein step (3c) is performed in DMAc.

Embodiment I-87. The process of Embodiment I-85 or I-86, further comprising isolating the compound of formula (33).

Embodiment I-88. A compound of formula (13), or a salt thereof,

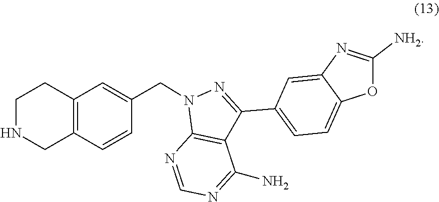 [SMILES: Nc1nc2cc(-c3nn(Cc4ccc5c(c4)CCNC5)c4ncnc(N)c34)ccc2o1]

Embodiment I-89. A compound of formula (13a),

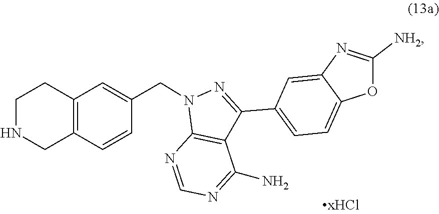

wherein n is 1, 2, or 3.

Embodiment I-90. The compound of Embodiment I-89, wherein n is 3.

Embodiment I-91. The compound of formula (13c),

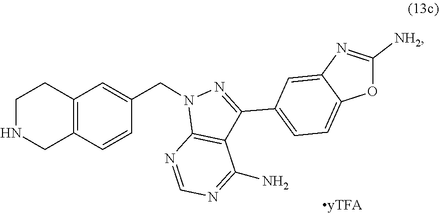

wherein y is 1, 2, or 3.

Embodiment I-92. The compound of Embodiment I-91, wherein y is 3.

Embodiment I-93. A compound of formula (32), or a salt thereof,

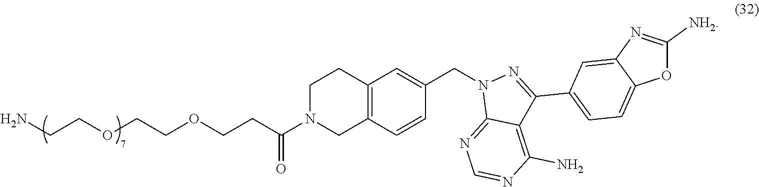

Embodiment II-1. A process for preparing a compound of formula (3), or a salt thereof, comprising:

(1a) contacting a compound of formula (1), or a salt thereof,

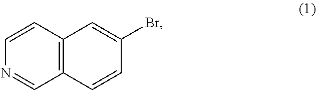 [SMILES: Brc1ccc2cnccc2c1]

with a reducing agent, to yield a compound of formula (2), or a salt thereof

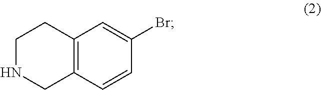 [SMILES: Brc1ccc2c(c1)CCNC2]

and

(2a) contacting a compound of formula (2), or a salt thereof, with an amino protecting group reagent to yield a compound of formula (3), or a salt thereof,

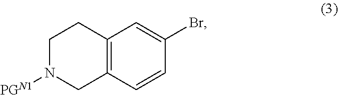

wherein PG^{N1} is an amino protecting group.

Embodiment II-2. The process of Embodiment II-1, wherein the reducing agent is sodium borohydride, and/or wherein step (1a) is performed in the presence of acetic acid.

Embodiment II-3. The process of any one of Embodiments II-1 to II-2, wherein:

(a) the amino protecting group reagent is triphenylmethyl chloride; and/or

(b) PG^{N}1 is triphenylmethyl (trityl).

Embodiment II-4. The process of any one of Embodiments II-1 to II-3, wherein:

(a) step (2a) is performed in the presence of an activating reagent, optionally wherein the activating reagent is 4-dimethylaminopyridine (DMAP); and/or

(b) step (2a) is performed in dichloromethane (DCM).

Embodiment II-5. The process of any one of Embodiments II-1 to II-4, further comprising isolating the compound of formula (3).

Embodiment II-6. The process of any one of Embodiments II-1 to II-5, further comprising:

(3a′) contacting the compound of formula (3), or a salt thereof, with an organometallic/metal reagent and formaldehyde to yield a compound of formula (5), or a salt thereof,

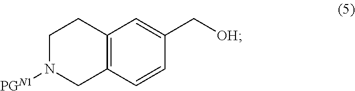

(3a) contacting the compound of formula (3), or a salt thereof, with an organometallic reagent and dimethylformamide (DMF) to yield a compound of formula (4), or a salt thereof,

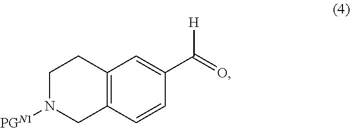

optionally wherein step (3a) is performed in tetrahydrofuran (THF), and/or wherein the organometallic reagent is an alkyl magnesium halide.

Embodiment II-7. The process of Embodiment II-6, further comprising

(4a) contacting the compound of formula (4), or a salt thereof, with a reducing agent to yield a compound of formula (5), or a salt thereof,

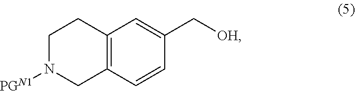

optionally wherein the reducing agent is sodium borohydride and/or wherein step (4a) is performed in a solvent selected from the group consisting of methanol, THF, and mixture thereof.

Embodiment II-8. The process of any one of Embodiments II-6 to II-7, further comprising

(5a) contacting the compound of formula (5), or a salt thereof, with a PG^{N}1 deprotecting reagent to yield a compound of formula (6), or a salt thereof,

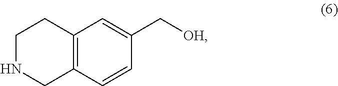 [SMILES: OCc1ccc2c(c1)CCNC2]

optionally wherein step (5a) is performed in DCM; and

(6a) contacting the compound of formula (6), or a salt thereof, with a Boc protecting group reagent to yield a compound of formula (7), or a salt thereof,

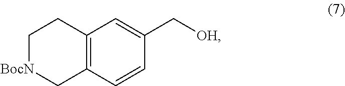 [SMILES: CC(C)(C)OC(=O)N1CCc2cc(CO)ccc2C1]

optionally wherein step (6a) is performed in THF, and/or wherein the process further comprises isolating the compound of formula (7).

Embodiment II-9. The process of Embodiment II-8, wherein:

(a) the PG^{N}1 deprotecting reagent is an acid; and/or

(b) the Boc protecting group reagent is Boc₂O.

Embodiment II-10. The process of any one of Embodiments II-8 to II-9, further comprising

(7a) contacting the compound of formula (7), or a salt thereof, with an alcohol activating reagent to yield a compound of formula (8), or a salt thereof,

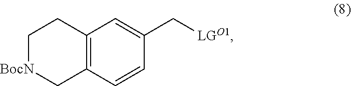

wherein −LG^{O1} is a leaving group, optionally wherein the process further comprises isolating the compound of formula (8).

Embodiment II-11. The process of Embodiment II-10, wherein:
(a) the alcohol activating reagent is a sulfonyl halide or a halogenating reagent, optionally methanesulfonyl chloride (mesyl chloride; CH₃SO₂Cl); and/or

(b) −LG^{O1} is a sulfonate ester or a halide, optionally mesylate (—O—SO₂CH₃).

Embodiment II-12. The process of any one of Embodiments II-10 to II-11, wherein:

(a) step (7a) is performed in the presence of a base, optionally wherein the base is diisopropylethylamine (DIPEA); and/or

(b) step (7a) is performed in DCM.

Embodiment II-13. The process of any one of Embodiments II-10 to II-12, further comprising

(8a) contacting the compound of formula (8), or a salt thereof, with a compound of formula (9), or a salt thereof,

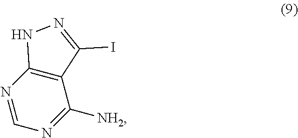 [SMILES: Nc1ncnc2[nH]nc(I)c12]

to yield a compound of formula (10), or a salt thereof,

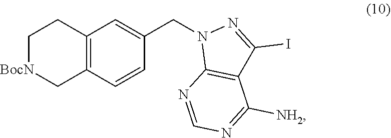 [SMILES: CC(C)(C)OC(=O)N1CCc2cc(Cn3nc(I)c4c(N)ncnc43)ccc2C1]

optionally wherein step (8a) is performed in DMF, and/or wherein the method further comprises isolating the compound of formula (10).

Embodiment II-14. The process of Embodiment II-13, further comprising (9a) contacting the compound of formula (10), or a salt thereof, with a compound of formula (11) or a salt thereof,

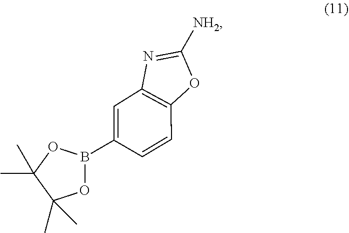 [SMILES: CC1(C)OB(c2ccc3oc(N)nc3c2)OC1(C)C]

to yield a compound of formula (12), or a salt thereof,

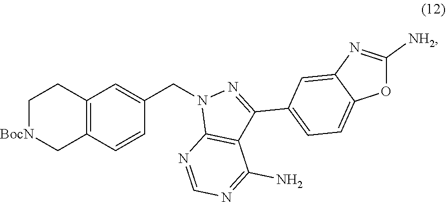 [SMILES: CC(C)(C)OC(=O)N1CCc2cc(Cn3nc(-c4ccc5oc(N)nc5c4)c4c(N)ncnc43)ccc2C1]

optionally wherein the process further comprises isolating the compound of formula (12).

Embodiment II-15. The process of Embodiment II-14, wherein the compound of formula (11) is prepared by borylation of a compound of formula (11a), or a salt thereof,

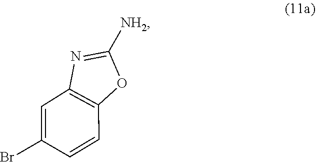 [SMILES: Nc1nc2cc(Br)ccc2o1]

optionally wherein borylation is performed with contact with a boronic ester reagent, further optionally wherein the boronic ester reagent is bis(pinacolato)diboron (B₂Pin₂).

Embodiment II-16. The process of any one of Embodiments II-14 to II-15, wherein:

(a) step (9a) is performed in the presence of a palladium catalyst, optionally wherein the palladium catalyst is Pd(PPh₃)₄; and/or

(b) step (9a) is performed in a solvent selected from the group consisting of water, dioxane, and mixture thereof.

Embodiment II-17. The process of any one of Embodiments II-14 to II-16, further comprising

(10a) contacting the compound of formula (12) with an acid to yield a compound of formula (13),

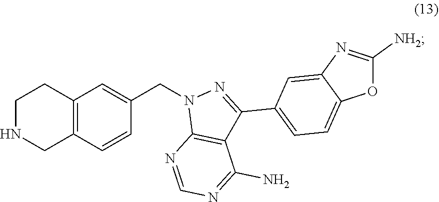 [SMILES: Nc1nc2cc(-c3nn(Cc4ccc5c(c4)CCNC5)c4ncnc(N)c34)ccc2o1]

and

(11a) preparing a salt of a compound of formula (13); optionally wherein step (10a) and step (11a) are performed in water.

Embodiment II-18. The process of Embodiment II-17, wherein:

(a) the acid is hydrochloric acid, thereby yielding a hydrochloric salt of compound of formula (13a),

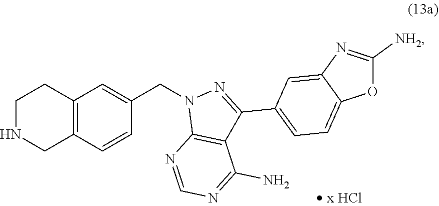

wherein x is 1, 2, or 3; or

(b) the acid is trifluoroacetic acid, thereby yielding a TFA salt of compound of formula (13c),

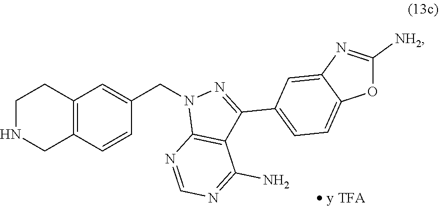

wherein y is 1, 2, or 3.

Embodiment II-19. The process of any one of Embodiments II-17 to II-18, further comprising isolating the compound of formula (13), (13a), or (13c).

Embodiment II-20. A process for preparing a compound of formula (21), or a salt thereof, comprising: (1b) contacting a compound of formula (20), or a salt thereof,

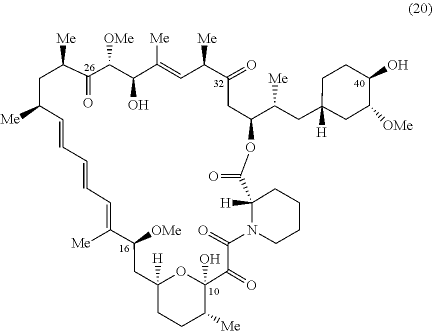 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]

with a hydroxyl protecting group reagent, to yield a compound of formula (21), or a salt thereof,

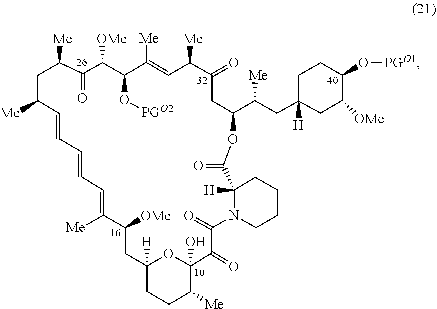

wherein PG^{O1} and PG^{O2} are the same or different at each instance a hydroxyl protecting group, optionally wherein step (1b) is performed in the presence of imidazole, and/or wherein step (1b) is performed in DCM.

Embodiment II-21. The process of Embodiment II-20, wherein:

(a) each hydroxyl protecting group reagent is triethylchlorosilane (TES-Cl);

(b) PG^{O1} is triethylsilyl ether (TES); and/or

(c) PG^{O2} is triethylsilyl ether (TES).

Embodiment II-22. The process of any one of Embodiments II-20 to II-21, further comprising isolating the compound of formula (21).

Embodiment II-23. The process of any one of Embodiments II-20 to II-22, further comprising:

(2b) contacting a compound of formula (21), or a salt thereof, with a reducing agent to yield a compound of formula (22), or a salt thereof,

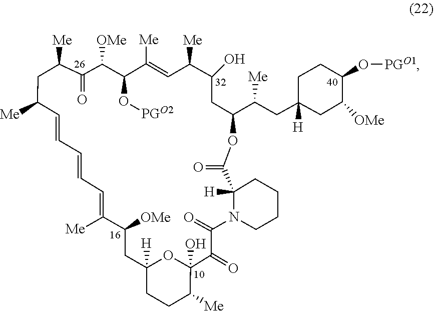

optionally wherein the reducing agent is LiAl(Ot-Bu)₃H, and/or wherein step (2b) is performed in THIF.

Embodiment II-24. The process of Embodiment II-23, wherein the product from step (2b) is subsequently contacted with Cu(OAc)₂.

Embodiment II-25. The process of any one of Embodiments II-23 to II-24, further comprising isolating the compound of formula (22).

Embodiment II-26. The process of any one of Embodiments II-23 to II-25, further comprising: (3b) contacting a compound of formula (22), or a salt thereof, with a PG^{O1} deprotecting reagent and a PG^{O2} deprotecting reagent to yield a compound of formula (23), or a salt thereof,

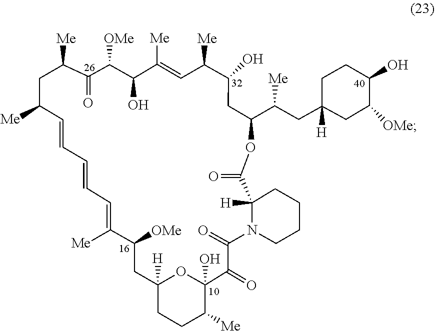 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

optionally wherein step (3b) is performed in THF, further optionally wherein the PG^{O1}

deprotecting reagent is an acid, or wherein the PG^{O2} deprotecting reagent is an acid.

Embodiment II-27. The process of Embodiment II-26, further comprising isolating the compound of formula (23).

Embodiment II-28. The process of any one of Embodiments II-26 to II-27, further comprising

(4b) contacting the compound of formula (23), or a salt thereof, with a compound of formula (24), or a salt thereof,

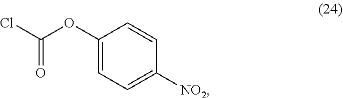 [SMILES: O=C(Cl)Oc1ccc([N+](=O)[O-])cc1]

to yield a compound of formula (25), or a salt thereof,

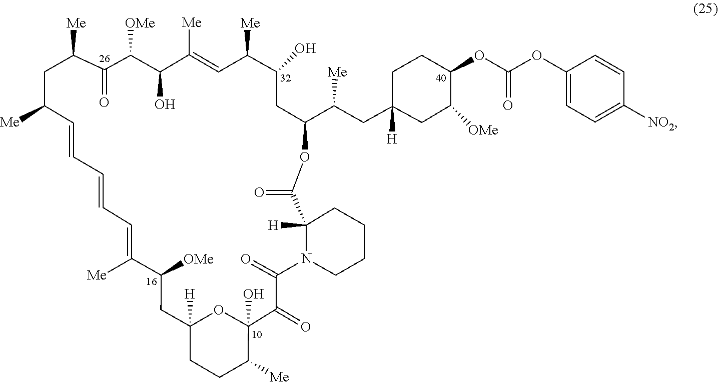 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

optionally wherein step (4b) is performed in DCM, and/or wherein the process further comprises isolating the compound of formula (25).

Embodiment II-29. A process for preparing a compound of formula (31), or a salt thereof, comprising: (1c) contacting a compound of formula (30), or a salt thereof,

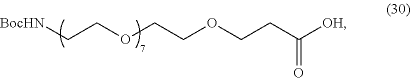

with a compound of formula (13), or a salt thereof,

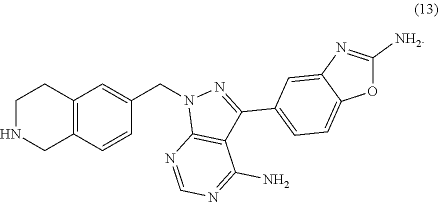 [SMILES: Nc1nc2cc(-c3nn(Cc4ccc5c(c4)CCNC5)c4ncnc(N)c34)ccc2o1]

to yield the compound of formula (31), or a salt thereof,

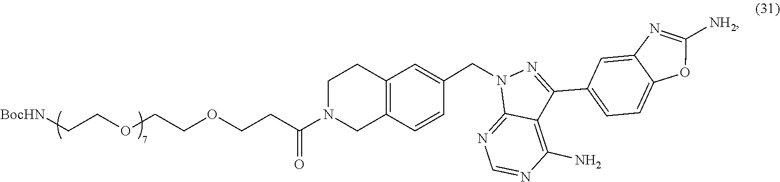

optionally wherein the process further comprises isolating the compound of formula (31).

Embodiment II-30. The process of Embodiment II-29, wherein the compound of formula (13), or a salt thereof, is:

(a) a compound of formula (13a),

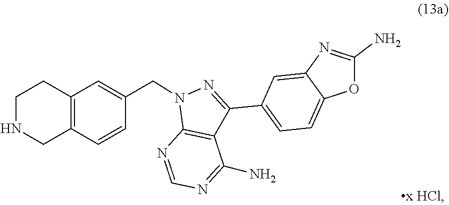

wherein n is 1, 2, or 3; or

(b) a compound of formula (13c),

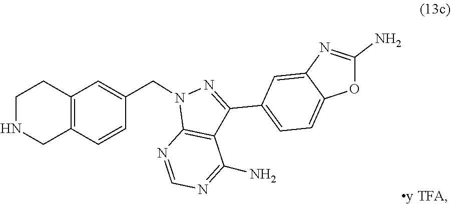

wherein y is 1, 2, or 3.

Embodiment II-31. The process of Embodiment II-29 or II-30, wherein:

(a) step (1c) is performed in the presence of a coupling reagent, optionally wherein the coupling reagent is 1-ethyl-3-(3-dimethylaminopropyl)carbodiimide (EDCI);

(b) step (1c) is performed in the presence of an activating reagent, optionally wherein the activating reagent is hydroxybenzotriazole (HOBt); and/or

(c) step (1c) is performed in dimethylacetamide (DMM).

Embodiment II-32. The process of any one of Embodiments II-29 to II-31, further comprising

(2c) contacting the compound of formula (31) with a Boc removing agent to yield a compound of formula (32), or a salt thereof,

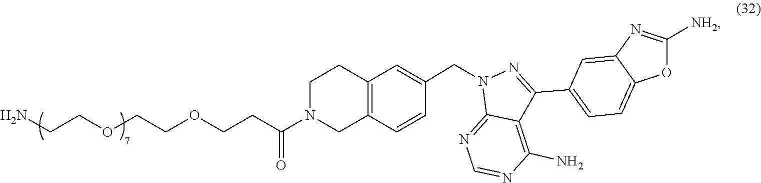

optionally wherein the Boc removing reagent is hydrochloric acid, and/or wherein step (2c) is performed in a solvent selected from the group consisting of water, DCM, dimethylacetamide (DMAc), and mixtures thereof.

Embodiment II-33. The process of Embodiment II-32, further comprising isolating the compound of formula (32).

Embodiment II-34. The process of any one of Embodiments II-32 to II-33, further comprising

(3c) contacting the compound of formula (32), or a salt thereof, with a compound of formula (25), or a salt thereof,

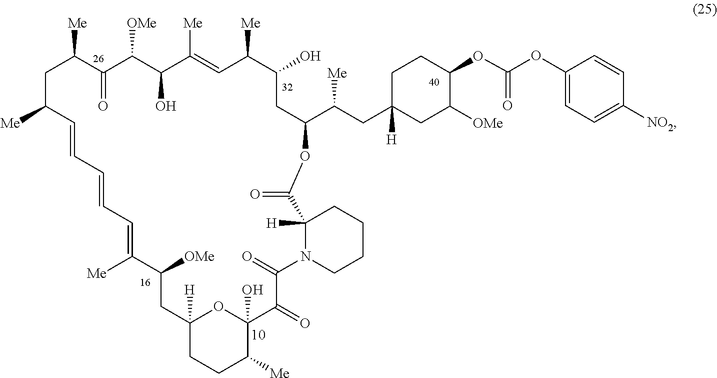 [SMILES: COC1C[C@H](C[C@@H](C)[C@@H]2C[C@@H](O)[C@H](C)/C=C(\C)[C@@H](O)[C@@H](OC)C(=O)[C@H](C)C[C@H](C)/C=C/C=C/C=C(\C)[C@@H](OC)C[C@@H]3CC[C@@H](C)[C@@](O)(O3)C(=O)C(=O)N3CCCC[C@H]3C(=O)O2)CC[C@H]1OC(=O)Oc1ccc([N+](=O)[O-])cc1]

to yield a compound of formula (33), or a salt thereof,

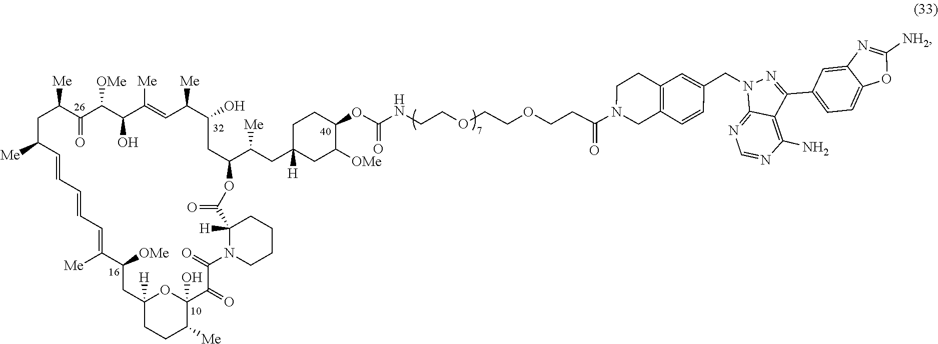

optionally wherein step (3c) is performed in DMAc, and/or wherein the process further comprises isolating the compound of formula (33).

Embodiment 11-35. A compound of:

(a) formula (13), or a salt thereof,

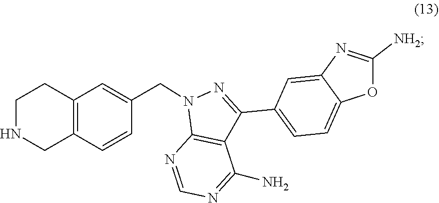 [SMILES: Nc1nc2cc(-c3nn(Cc4ccc5c(c4)CCNC5)c4ncnc(N)c34)ccc2o1]

(b) formula (13a),

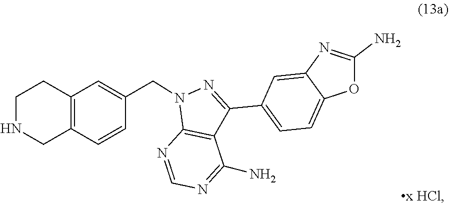

wherein is 1, 2, or 3;

(c) formula (13c),

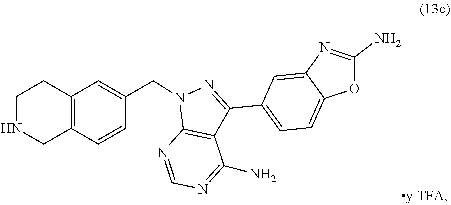

wherein n is 1, 2, or 3; or

(d) formula (132), thereof,

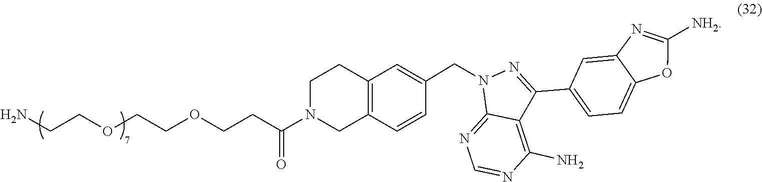

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure or scope of the appended claims.

The following abbreviations have the following meanings unless otherwise indicated and any other abbreviations used herein and not defined have their standard generally accepted meaning:
Ac: acetate
ACN and MeCN: acetonitrile
Boc: tert-butoxycarbonyl
Boc₂O: di-tert-butyl dicarbonate or Boc anhydride
ca.: approximately
DCM: dichloromethane
DI: deionized water
DMAP: 4-dimethylaminopyridine
DMF: dimethylformamide
DSC: differential scanning calorimetry
EtOAc: ethyl acetate
EtOH: ethanol
GC: gas chromatography
IPA: isopropyl alcohol
IPAc: isopropyl acetate
h or hr: hour(s)
HCl: hydrochloric acid
HPLC: high performance liquid chromatography
LC/MS: liquid chromatography/mass spectrometry
MeOH: methanol
MEK: methyl ethyl ketone
MIBK: methyl isobutyl ketone
min: minute(s)
NaOH: sodium hydroxide
ppm: parts per million
RT or rt: room temperature
TBME: tert-butyl methyl ether
TEA and Et3N: triethylamine
TFA: trifluoroacetic acid
TGA: thermogravimetric analysis
THF: tetrahydrofuran
UV: ultraviolet
v/v: volume by volume
vol or vols: volume(s)
% w/w: weight for weight percent
wt: weight

### Example 1—Synthetic Protocol for Compound 13b′

Detailed below is a general synthetic protocol for Compound 13b′.

### Synthesis of Compound 13b′-5-(4-amino-1-((1,2,3,4-tetrahydroisoquinolin-6-yl)methyl)-1H-pyrazolo[3,4-d]pyrimidin-3-yl)benzo[d]oxazol-2-amine tris HCl

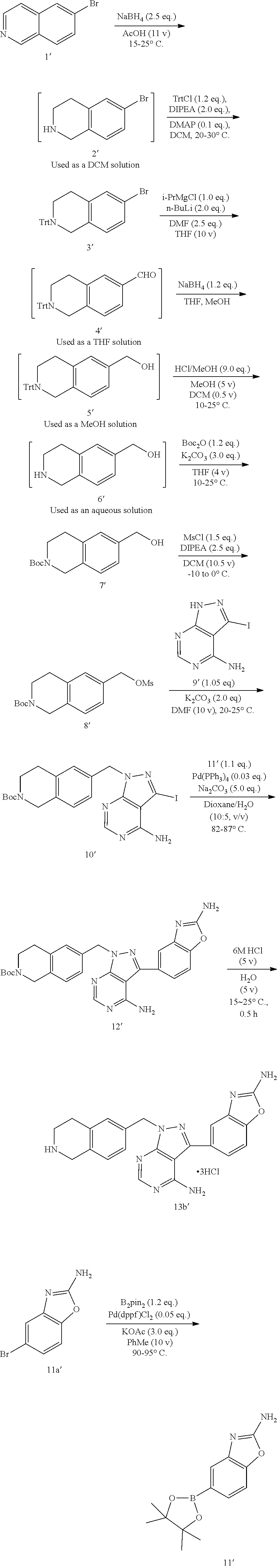

### Part 1-Synthesis of Compound 2′

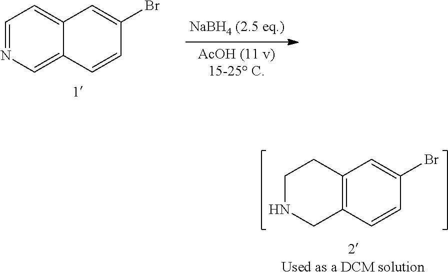

To a 100-L reactor, with agitation, was charged 6-bromoisoquinoline (1′) (4.65 kg, 22.4 mol, 1.0 eq.) and AcOH (52 L). The reactor was purged with nitrogen three times. The mixture was maintained at 20-30° C. for 5 min and then cooled to 10-15° C. To this was charged NaBH₄ (2.11 kg, 55.9 mol, 2.5 eq.) in 14 portions over a 160 min period, maintaining temperature at 15-25° C. The resulting mixture was maintained at 10-20° C. for 15 min at which point HPLC monitoring showed reaction completion.

The reaction was then slowly charged to ice-water (93 L) over 1 h, maintaining temperature at 5-15° C. The resulting mixture was cooled to below 5° C. To this was charged 8 N aqueous NaOH (121 L) dropwise, adjusting pH to 12-14 over 1 h. This mixture was extracted with DCM (47L×2). The combined organic phases were washed with brine (47 L), dried over anhydrous Na₂SO₄ (15 kg) for 1 h, and filtered, washing the cake with DCM (20 L). The filtrate was then partially concentrated under reduced pressure at 35-40° C. (to ca. 20 L). This DCM solution of 6-bromo-1,2,3,4-tetrahydroisoquinoline (2′) was used directly in the next step.

TABLE 1
HPLC Method for Part 1 of Example 1
HPLC method:

| Instrument: | Agilent 1260 series HPLC | |
|---|---|---|
| Column: | Agilent Poroshell 120 EC-C18 | |
| | (4.6 × 100 mm, 2.7 μm) | |
| Mobile Phase: | A: 0.1% aq. H₃PO₄ | |
| | B: MeCN | |
| Gradient: | Time (min) | B (%) |
| | 0.00 | 5 |
| | 10.00 | 90 |
| | 15.00 | 90 |

| | |
|---|---|
| | Post time: 3 min |
| Flow Rate: | 1.0 mL/min |
| UV Detector Wavelength: | 210 nm |
| Column Temperature: | 30° C. |
| Run Time: | 15.0 min |
| Retention Times: | 6-bromoisoquinoline (1'): 4.2 min |
| | 6-bromo-1,2,3,4-tetra- |
| | hydroisoquinoline (2'): 4.6 min |

¹H NMR (400 MHz, CDCl₃) δ 7.24 (br s, 2H), 6.88 (d, J=8 Hz, 1H), 3.95 (s, 2H), 3.12 (t, J=6 Hz, 2H), 2.98 (br s, 1H), (2.78 (t, J=6 Hz, 2H).

### Part 2: Synthesis of Compound 3′

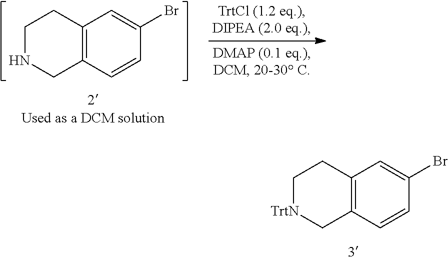

### Compound 3′-6-bromo-2-trityl-1,2,3,4-tetrahydroisoquinoline

To a 100-L reactor, with agitation, was charged a DCM solution of 6-bromo-1,2,3,4-tetrahydroisoquinoline (2′) (23.9 kg, 22.4 mol, 1.0 eq.), DIPEA (5.8 kg, 44.9 mol, 2.0 eq.), and DMAP (273 g, 2.24 mol, 0.1 eq.) under N2 protection. This was maintained at 10-30° C. for 10 min affording a clear solution. The solution was then cooled to 0-10° C. To this was charged a DCM solution of trityl chloride (TrtCl) (7.4 kg in DCM, 23 L, 26.5 mol, 1.2 eq.) dropwise at 0-10° C. over 0.5 h. The resulting mixture was maintained at 20-30° C. for 0.5 h at which point HPLC monitoring showed reaction completion.

To the mixture was then charged 1 N aqueous NaOH (10 L) dropwise at 5-15° C. over 0.5 h. The phases were separated and the aqueous phase extracted with DCM (12.3 kg×1). The combined organic phases were washed with aqueous. citric acid (5 wt %, 36L×3), washed with brine (36 L), and then partially concentrated under reduced pressure at 35-40° C. (to ca. 10-15 L).

To the partially concentrated residue was charged EtOAc (22 kg) the resulting mixture was partially concentrated under reduced pressure at 35-40° C. (to ca. 15-20 L). This solvent swap was performed a total of three times, affording solid precipitate. To the mixture was then charged EtOAc (12 kg). This mixture was cooled to 10-15° C. and maintained at 10-15° C. for 0.5 h. The resulting mixture was filtered, washing the cake with EtOAc (10 kg×3).

The filtrate was partially concentrated under reduced pressure at 35-40° C. (to ca. 10-15 L). To this was charged n-heptane (10 L) dropwise at 20-30° C. affording solid precipitate. This mixture was maintained at at 20-30° C. for 0.5 h and then cooled to and maintained at 5-10° C. for 0.5 h. The resulting mixture was filtered, washing the cake with n-heptane (10 L). The wet cake was dried under reduced pressure at 45-50° C. overnight to produce 7.4 kg of 6-bromo-2-trityl-1,2,3,4-tetrahydroisoquinoline (3′) as an off-white solid (73% uncorrected yield from 6-bromoisoquinoline (1′)).

After concentrating mother liquor to dryness, ca. 2.9 kg of 6-bromo-2-trityl-1,2,3,4-tetrahydroisoquinoline (3′) was obtained. This was purified by silica gel column chromatography yielding 744 g of 6-bromo-2-trityl-1,2,3,4-tetrahydroisoquinoline (3′). Subsequent crystallization from EtOAc/n-heptane (1:2.5, v/v) gave 663 g of 6-bromo-2-trityl-1,2,3,4-tetrahydroisoquinoline (3′) as an off-white solid.

Combination of solids afforded 8.4 kg 6-bromo-2-trityl-1,2,3,4-tetrahydroisoquinoline (3′) (77% uncorrected yield from 6-bromoisoquinoline (1′)).

TABLE 2
HPLC Method for Part 2 of Example 1
HPLC method:

| Instrument: | Agilent 1260 series HPLC | |
|---|---|---|
| Column: | XBridge C18 (3.0 × 150 mm, 3.5 μm) | |
| Mobile Phase: | A: 10 M aq. NH₄OAc | |
| | B: MeCN | |
| Gradient: | Time (min) | B (%) |
| | 0.00 | 5 |
| | 10.00 | 90 |
| | 20.00 | 90 |

| | |
|---|---|
| | Post time: 5 min |
| Flow Rate: | 0.6 mL/min |
| UV Detector Wavelength: | 210 nm |
| Column Temperature: | 30° C. |
| Run Time: | 20.0 min |
| Retention Times: | 6-bromoisoquinoline (1'): 8.2 min |
| | 6-bromo-1,2,3,4-tetrahydroisoquinoline |
| | (2'): 5.5 min |
| | TrtCl: 10.6 min |
| | 6-bromo-2-trityl-1,2,3,4-tetrahydro- |
| | isoquinoline (3'): 15.0 min |

¹H NMR (400 MHz, CDCl₃) δ 7.54-7.52 (m, 6H), 7.28-7.25 (m, 7H), 7.19-7.14 (m, 4H), 6.78 (d, J=8 Hz, 1H), 3.37 (br s, 2H), 2.97 (br s, 2H), 2.52 (br s, 2H).

### Part 3: Synthesis of Compound 4′

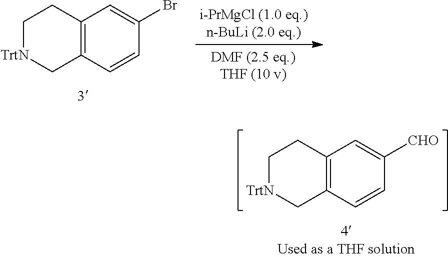

### Compound 4′-2-trityl-1,2,3,4-tetrahydroisoquinoline-6-carbaldehyde

To a 30-L reactor, with agitation, was charged 6-bromo-2-trityl-1,2,3,4-tetrahydroisoquinoline (3′) (7 kg, 15.4 mol, 1.0 eq.) and THE (12.6 kg). The reactor was purged with nitrogen three times. The mixture was maintained at 20-25° C. for 10 min affording a clear solution. Separately, to a 100-L reactor, with agitation, was charged THF (50.4 kg). The reactor was purged with nitrogen three times. To this was charged 2.0 M i-PrMgCl in THE (7.7 L, 15.4 mol, 1.0 eq.) dropwise at 20-25° C. under nitrogen protection. The resulting mixture was then cooled to and maintained at −35-−25° C. under N2 protection. To this was charged 2.5 M n-BuLi in n-hexane (12.4 L, 30.8 mol, 2.0 eq.) dropwise −35-−25° C. under N₂ protection and then the THF solution of 6-bromo-2-trityl-1,2,3,4-tetrahydroisoquinoline (3′) (7.0 kg, 15.4, 1.0 eq.) dropwise over a period of 1 h at −35-−25° C. The resulting mixture was maintained at −35-−25° C. for 10 min at which point HPLC monitoring showed reaction completion.

To the reaction was then charged DMF (2.9 kg, 38.5 mol, 2.5 eq.) dropwise over a period of 30 min at −35-−25° C. This was maintained at −35° C.-−25° C. for 10 min at which point the mixture was charged into saturated aqueous NH₄C₁ (70 kg) at below 10° C. and maintained for 30 min. The phases were separated and the aqueous phase extracted with EtOAc (32 kg×2). The combined organic phases were washed with brine (32 kg) and concentrated to dryness under reduced pressure at 40-45° C.

The resulting residue was dissolved in THE (16 kg) and partially concentrated under reduced pressure at 40-45° C. (to ca. 10-15 L solution). This THE charge and partial concentration was repeated two more times. To the THF solution was charged THF (45 kg), affording about 70 L THF solution of 2-trityl-1,2,3,4-tetrahydroisoquinoline-6-carbaldehyde (4′). The solution was used in the next step directly.

TABLE 3
HPLC Method for Part 3 of Example 1
HPLC method:

| Instrument: | Agilent 1260 series HPLC | |
|---|---|---|
| Column: | XBridge C18 (3.0 × 150 mm, 3.5 μm) | |
| Mobile Phase: | A: 10 mM aq. NH₄OAc | |
| | B: MeCN | |
| Gradient: | Time (min) | B (%) |
| | 0.00 | 5 |
| | 10.00 | 90 |
| | 20.00 | 90 |

| | |
|---|---|
| | Post time: 5 min |
| Flow Rate: | 0.6 mL/min |
| UV Detector Wavelength: | 210 nm |
| Column Temperature: | 30° C. |
| Run Time: | 20.0 min |
| Retention Times: | 6-bromoisoquinoline (1'): 8.2 min |
| | 6-bromo-1,2,3,4-tetrahydroisoquinoline |
| | (2'): 5.5 min |
| | TrtCl: 10.6 min |
| | 6-bromo-2-trityl-1,2,3,4-tetrahydro- |
| | isoquinoline (3'): 15.0 min |
| | 2-trityl-1,2,3,4-tetrahydroisoquinoline- |
| | 6-carbaldehyde (4'): 13.1 min |

¹H NMR (400 MHz, CDCl₃) δ 9.93 (s, 1H), 7.64-7.53 (m, 7H), 7.33-7.25 (in, 7H), 7.19-7.16 (m, 3H), 7.07 (d, J=8 Hz, 1H), 3.52 (br s, 2H), 3.08 (br s, 2H), 2.58 (br s, 2H).

### Part 4: Synthesis of Compound 5′

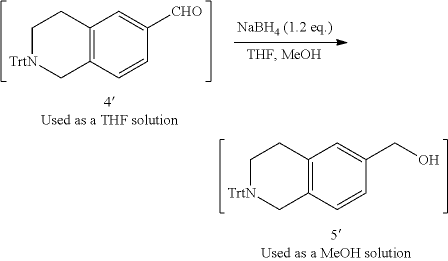

### Compound 5″-(2-trityl-1,2,3,4-tetrahydroisoquinolin-6-yl)methanol

To a 100-L reactor, with agitation, was charged a THF solution of 2-trityl-1,2,3,4-tetrahydroisoquinoline-6-carbaldehyde (4′) (about 70 L from last step, ca. 15.4 mol) and MeOH (14 L). The reactor was purged with nitrogen three times. The mixture was maintained at 20-25° C. for 10 min and then cooled to −5-0° C. under nitrogen protection. To this was charged NaBH₄ (700 g, 18.5 mol, 1.2 eq.) in 15 portions over a period of 120 min at −5-0° C. This was maintained at −5-0° C. for 10 min at which point HPLC monitoring showed reaction completion.

The reaction mixture was then charged into saturated aqueous NH₄C₁ (70 kg) at below 10° C. and then maintained for 30 min at room temperature. The phases were separated and the aqueous phase extracted with EtOAc (32 kg×2). The combined organic phases were washed with brine (32 kg) and partially concentrated under reduced pressure at 40-45° C. (to ca. 10-15 L). The resulting material was dissolved in MeOH (30 kg) and partially concentrated under reduced pressure at 40-45° C. (to ca. 10-15 L). This MeOH charge and partial concentration was repeated once more. To the MeOH solution was charged MeOH (20 kg) and afford about 35 L MeOH solution of (2-trityl-1,2,3,4-tetrahydroisoquinolin-6-yl)methanol (5′). The solution was used in the next step directly.

TABLE 4
HPLC Method for Part 4 of Example 1
HPLC method:

| Instrument: | Agilent 1260 series HPLC | |
|---|---|---|
| Column: | XBridge C18 (3.0 × 150 mm, 3.5 μm) | |
| Mobile Phase: | A: 10 mM aq. NH₄OAc | |
| | B: MeCN | |
| Gradient: | Time (min) | B (%) |
| | 0.00 | 5 |
| | 10.00 | 90 |
| | 20.00 | 90 |

| | |
|---|---|
| | Post time: 5 min |
| Flow Rate: | 0.6 mL/min |
| UV Detector Wavelength: | 210 nm |
| Column Temperature: | 30° C. |
| Run Time: | 20.0 min |
| Retention Times: | 2-trityl-1,2,3,4-tetrahydroisoquinoline- |
| | 6-carbaldehyde (4'): 13.1 min |
| | (2-trityl-1,2,3,4-tetrahydroisoquinolin- |
| | 6-yl)methanol (5'): 12.1 min |

¹H NMR (400 MHz, CDCl₃) δ 7.55-7.53 (m, 6H), 7.27-7.23 (m, 6H), 7.16-7.12 (m, 4H), 7.05 (d, J=8 Hz, 1H), 6.89 (d, J=8 Hz, 1H), 4.59 (s, 2H), 3.45 (br s, 2H), 3.00 (br s, 2H), 2.53 (br s, 2H).

### Part 5-Synthesis of Compound 6′

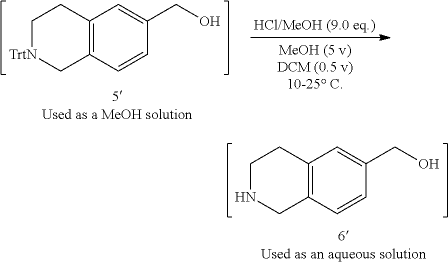

### Compound 6′-(1,2,3,4-tetrahydroisoquinolin-6-yl)methanol

To a 100-L reactor, with agitation, was charged a MeOH solution of (2-trityl-1,2,3,4-tetrahydroisoquinolin-6-yl)methanol (5′) (about 35 L from last step, ca. 15.4 mol) and DCM (3.5 L). The reactor was purged with nitrogen three times. The mixture was maintained at 20-25° C. for 10 min affording a clear solution. This was cooled to 0-5° C. under N₂ protection. To this mixture was charged HCl in MeOH (ca. 10 M, ca. 14 L, ca. 140 mol, 9 eq.) (made by bubbling HCl gas (5.2 kg, 140 mol, 9 eq.) slowly into MeOH (11 kg) at below 0° C. over a period of ca. 3 h) dropwise at 0-10° C. under N₂ protection over 60 min. The mixture was warmed to 20° C. and maintained at 20-30° C. for 1 h at which point HPLC monitoring showed reaction completion.

The mixture was partially concentrated under reduced pressure at 35-40° C. (to ca. 5 L) then cooled to room temperature. The concentrate was then dissolved in water (70 kg) and to this was charged MTBE (26 kg). This was maintained at room temperature for 10 min. The phases were separated and the aqueous phase extracted with MTBE (26 kg×2) affording about 70 L of an aqueous solution of (1,2,3,4-tetrahydroisoquinolin-6-yl)methanol (6′). The solution of was used directly in the next step.

TABLE 5
HPLC Method for Part 5 of Example 1
HPLC method:

| Instrument: | Agilent 1260 series HPLC | | |
|---|---|---|---|
| Column: | Agilent Bonus RP (4.6 × 100 mm, 2.7 μm) | | |
| Mobile Phase: | A: 10 mM aq. NH₄HCO₃ | | |
| | B: MeCN | | |
| | D: MeOH | | |
| | Time (min) | B (%) | D (%) |
| Gradient: | 0.00 | 5 | 5 |
| | 10.00 | 85 | 5 |
| | 15.00 | 85 | 5 |
| | 20.00 | 5 | 5 |

| | Post time: 3 min |
|---|---|
| Flow Rate: | 0.6 mL/min |
| UV Detector | 210 nm |
| Wavelength: | |
| Column | 30° C. |
| Temperature: | |
| Run Time: | 20.0 min |
| Retention Times: | (2-trityl-1,2,3,4-tetrahydroisoquinolin-6-yl)methanol |
| | (5′): 13.7 min |
| | (1,2,3,4-tetrahydroisoquinolin-6-yl)methanol |
| | (6′): 4.8 min |
| | TrtOH: 12.0 min |
| | TrtOMe: 13.2 min |

¹H NMR (400 MHz, CD₃OD) δ7.29-7.17 (m, 3H), 4.58 (s, 2H), 4.34 (s, 2H), 3.49 (t, J=6 Hz, 2H), 3.12 (t, J=6 Hz, 2H).

### Part 6-Synthesis of Compound 7′

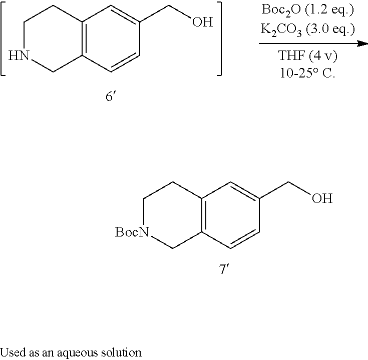

### Compound 7′-tert-butyl 6-(hydroxymethyl)-3,4-dihydroisoquinoline-2(1H)-carboxylate

To a 200-L reactor, with agitation, was charged an aqueous solution of (1,2,3,4-tetrahydroisoquinolin-6-yl)methanol (6′) (about 70 L, ca. 15.4 mol). This was cooled to below 15° C. The pH was adjusted to 7-8 with solid K₂CO₃ (7-8 kg). To this was then charged additional K₂CO₃ (6.4 kg, 46.2 mol, 3.0 eq.) in portions and this was maintained for 5 minutes. To the resulting mixture was charged THE (13 kg) and this was maintained for 5 min. This resulting mixture was then cooled to below 10° C. and to this was charged a THE solution of (Boc)₂O (4.05 kg in 13 kg of THF, 18.5 mol, 1.2 eq.) dropwise below 10° C. over a period of 30 min. The temperature was allowed to rise to ambient temperature at a natural rate and then maintained at 10-25° C. for 30 min at which point HPLC monitoring showed reaction completion.

To the mixture was charged EtOAc (32 kg) and this was maintained for 5 min. The phases were separated and the aqueous phases extracted with EtOAc (32 kg). The combined organic phases were washed with brine (32 kg) and partially concentrated under reduced pressure at 40-45° C. (to ca. 10-15 L). To this was charged n-heptane (24 kg) and the resulting mixture was partially concentrated under reduced pressure at 40-45° C. (to 10-15 L). This n-heptane charge and partial concentrated was repeated twice more. The the final concentrate was charged n-heptane (10 kg) and this was maintain at room temperature for 30 min affording solid precipitate. The mixture was cooled to 5-10° C. and maintained at 5-10° C. for 30 min. This was then filtered, washing the cake with n-heptane (10 kg×2). The wet cake was dried at 40-45° C. under reduced pressure to constant weight to afford ca. 2.55 kg of tert-butyl 6-(hydroxymethyl)-3,4-dihydroisoquinoline-2(1H)-carboxylate (7′) (6300 uncorrected yield from 6-bromo-2-trityl-1,2,3,4-tetrahydroisoquinoline (3′)).

TABLE 6
HPLC Method for Part 6 of Example 1
HPLC method:

| Instrument: | Agilent 1260 series HPLC | | |
|---|---|---|---|
| Column: | Agilent Bonus RP (4.6 × 100 mm, 2.7 μm) | | |
| Mobile Phase: | A: 10 mM aq. NH₄HCO₃ | | |
| | B: MeCN | | |
| | D: MeOH | | |
| | Time (min) | B (%) | D (%) |
| Gradient: | 0.00 | 5 | 5 |
| | 10.00 | 85 | 5 |
| | 15.00 | 85 | 5 |
| | 20.00 | 5 | 5 |

| | |
|---|---|
| Flow Rate: | 0.6 mL/min |
| UV Detector | 210 nm |
| Wavelength: | |
| Column | 30° C. |
| Temperature: | |
| Run Time: | 20.0 min |
| Retention Times: | (2-trityl-1,2,3,4-tetrahydroisoquinolin-6-yl)methanol |
| | (5′): 13.7 min |
| | (1,2,3,4-tetrahydroisoquinolin-6-yl)methanol (6′): 4.8 |
| | min |
| | TrtOH: 12.0 min |
| | TrtOMe: 13.2 min |
| | tert-butyl 6-(hydroxymethyl)-3,4-dihydroisoquinoline- |
| | 2(1H)-carboxylate (7′): 9.9 min |

¹H NMR (400 MHz, CDCl₃) δ7.18-7.07 (m, 3H), 4.64 (s, 2H), 4.55 (s, 2H), 3.63 (t, J=6 Hz, 2H), 2.82 (t, J=6 Hz, 2H), 2.03 (br s, 1H), 1.49 (s, 9H).

### Part 7-Synthesis of Compound 8′

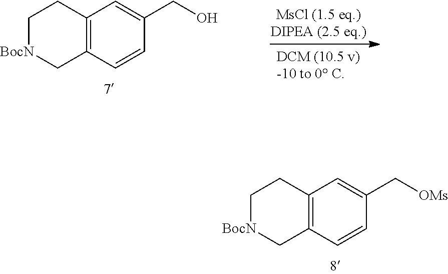 [SMILES: CC(C)(C)OC(=O)N1CCc2cc(CO)ccc2C1 ; CC(C)(C)OC(=O)N1CCc2cc(COS(C)(=O)=O)ccc2C1]

### Compound 8′-tert-butyl 6-(((methylsulfonyl)oxy)methyl)-3,4-dihydroisoquinoline-2(1h)-carboxylate

To a 50-L reactor, with agitation, was charged tert-butyl 6-(hydroxymethyl)-3,4-dihydroisoquinoline-2(1H)-carboxylate (7′) (2.1 kg, 7.97 mol, 1.0 eq.) and DCM (28 kg). The reactor was purged with nitrogen three times. The mixture was cooled to −10-0° C. To this was charged DIPEA (2.6 kg, 19.93 mol, 2.5 eq.) dropwise at −10-0° C. over 10 min. The resulting mixture was maintained at −10-0° C. for 5 min and then cooled to −15 to −5° C. To this was charged MsCl (1.37 kg, 11.96 mol, 1.5 eq.) in DCM (1.4 kg) slowly over a period of 90 min at −10-0° C. This was maintained at −10-0° C. for 30 min at which point HPLC monitoring showed reaction completion.

To the mixture was charged water (21 kg) slowly at below 5° C. The phases were separated and the aqueous phase extracted with DCM (14 kg). The combined organic phases were washed with water (11 kg), washed with brine (11 kg), dried over anhydrous Na₂SO₄ (4 kg) for 30 min. This was then filtered, washing the cake with DCM (4 L and then 2 L). The filtrate was concentrated to near dryness at below 35° C. under reduced pressure (ca. 3 L). This was cooled to room temperature and protected with nitrogen to afford 3.6 kg of crude tert-butyl 6-(((methylsulfonyl)oxy)methyl)-3,4-dihydroisoquinoline-2(1H)-carboxylate (8′). This was used directly in the next step without further purification.

TABLE 7
HPLC Method for Part 7 of Example 1
HPLC method:

| Instrument: | Agilent 1260 series HPLC | |
|---|---|---|
| Column: | Agilent Poroshell 120 EC-C18 | |
| | (4.6 × 100 mm, 2.7 μm) | |
| Mobile Phase: | A: 0.1% aq. H₃PO₄ | |
| | B: MeCN | |
| | Time (min) | B (%) |
| Gradient: | 0.00 | 5 |
| | 7.00 | 90 |
| | 12.00 | 90 |

| | Post time: 3 min |
|---|---|
| Flow Rate | 1.0 mL/min |
| UV Detector | 210 nm |
| Wavelength: | |
| Column | 30° C. |
| Temperature: | |
| Retention Times: | tert-butyl 6-(hydroxymethyl)-3,4-dihydroisoquinoline- |
| | 2(1H)-carboxylate (7′): 6.4 min |
| | tert-butyl 6-(((methylsulfonyl)oxy)methyl)-3,4- |
| | dihydroisoquinoline-2(1H)-carboxylate (8′): 4.8 min |
| | (this is the Me₂NH derivative of tert-butyl 6- |
| | (((methylsulfonyl)oxy)methyl)-3,4- |
| | dihydroisoquinoline-2(1H)-carboxylate (8′) |

¹H NMR (400 MHz, CDCl₃) δ 7.27-7.14 (m, 3H), 5.20 (s, 2H), 4.58 (s, 2H), 3.65 (br s, 2H), 2.94 (s, 3H), 2.85 (t, J=6 Hz, 2H), 1.49 (s, 9H).

### Part 8-Synthesis of Compound 10′

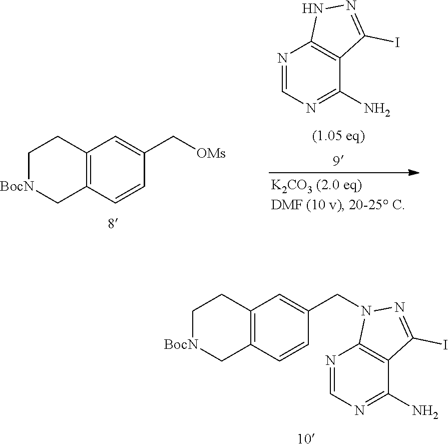 [SMILES: Nc1ncnc2[nH]nc(I)c12 ; CC(C)(C)OC(=O)N1CCc2cc(COS(C)(=O)=O)ccc2C1 ; CC(C)(C)OC(=O)N1CCc2cc(Cn3nc(I)c4c(N)ncnc43)ccc2C1]

### Compound 10′-tert-butyl 6-((4-amino-3-iodo-1H-pyrazolo[3,4-d]pyrimidin-1-yl)methyl)-3,4-dihydroisoquinoline-2(1H)-carboxylate

To a 100-L reactor, with agitation, was charged 3-iodo-1H-pyrazolo[3,4-d]pyrimidin-4-amine (9′) (2.19 kg, 8.37 mol, 1.05 eq.), DMF (26 kg), and K₂CO₃ (2.2 kg, 15.94 mol, 2.0 eq.). The reactor was purged with nitrogen three times. The resulting mixture was then cooled to 5-10° C. To this mixture was charged a DMF solution of tert-butyl 6-((methylsulfonyloxy)methyl)-3,4-dihydroisoquinoline-2(1H)-carboxylate (8′) (3.6 kg from previous step, ca. 7.97 mol) and DMF (26 kg) slowly at 5-10° C. over a period of about 25 min. The resulting mixture was then warmed to 15-20° C. and maintained at 15-20° C. for 14 h at which point HPLC monitoring showed reaction completion.

The mixture was slowly charged into ice-water (54 kg) and maintained at 15-20° C. for 30 min. This was then filtered, washing the cake with water (13.5 kg×3). The wet cake was slurried in MeOH (10.4 kg) at 65-70° C. for 30 min and then cooled to 10-15° C. and maintained at 10-15° C. for 0.5 h. This mixture was then filtered, washing the cake with MeOH (3.3 kg×2). The wet cake was dried to constant weight under reduced pressure at 45-50° C. to afford 2.9 kg of tert-butyl 6-((4-amino-3-iodo-1H-pyrazolo[3,4-d]pyrimidin-1-yl)methyl)-3,4-dihydroisoquinoline-2(1H)-carboxylate (10′) as a light-brown colored solid (73% yield from tert-butyl 6-(hydroxymethyl)-3,4-dihydroisoquinoline-2(1H)-carboxylate (7′)).

TABLE 8
HPLC Method for Part 8 of Example 1
HPLC method:

| Instrument: | Agilent 1260 series HPLC | |
|---|---|---|
| Column: | Agilent Poroshell 120 EC-C18 (4.6 × 100 mm, 2.7 μm) | |
| Mobile Phase: | A: 0.1% aq. H₃PO₄ | |
| | B: MeCN | |
| | Time (min) | B (%) |
| Gradient: | 0.00 | 5 |
| | 7.00 | 90 |
| | 12.00 | 90 |

| | Post time: 3 min |
|---|---|
| Flow Rate: | 1.0 mL/min |
| UV Detector | 210 nm |
| Wavelength: | |
| Column | 30° C. |
| Temperature: | |
| Run Time: | 20.0 min |
| Retention Times: | 3-iodo-1H-pyrazolo[3,4-d]pyrimidin-4-amine (9′): 2.4 |
| | min |
| | tert-butyl 6-((methylsulfonyloxy)methyl)-3,4- |
| | dihydroisoquinoline-2(1H)-carboxylate (8′): 6.5 min, |
| | 7.3 min, and 8.5 min (these are analysis artifacts as tert- |
| | butyl 6-((methylsulfonyloxy)methyl)-3,4- |
| | dihydroisoquinoline-2(1H)-carboxylate (8′) is degraded |
| | in the mobile phase) |
| | tert-butyl 6-((4-amino-3-iodo-1H-pyrazolo[3,4- |
| | d]pyrimidin-1-yl)methyl)-3,4-dihydroisoquinoline- |
| | 2(1H)-carboxylate (10′): 6.8 min |

¹H NMR (400 MHz, DMSO-d₆) δ 8.24 (br s, 1H), 7.09-7.04 (m, 3H), 5.41 (br s, 2H), 4.43 (br s, 2H), 3.50 (br s, 2H), 2.71 (br s, 2H), 1.40 (s, 9H).

### Part 9-Synthesis of Compound 11′

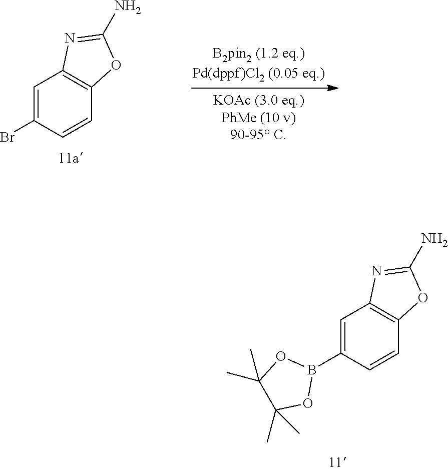 [SMILES: Nc1nc2cc(Br)ccc2o1 ; CC1(C)OB(c2ccc3oc(N)nc3c2)OC1(C)C]

### Compound 11′-5-(4,4,5,5-tetramethyl-1,3,2-dioxaborolan-2-yl)benzo[d]oxazol-2-amine

To a 100-L reactor, with agitation, was charged 5-bromobenzo[d]oxazol-2-amine (11a′) (3.3 kg, 15.58 mol, 1.0 eq.), PhMe (33 L), bis(pinacolato)diboron (4.75 kg, 18.7 mol, 1.2 eq.), and KOAc (4.59 kg, 46.75 mol, 3.0 eq.). This was maintained at room temperature for 5 min and the reactor was purged with nitrogen three times. To this was charged Pd(dppf)Cl₂ (570 g, 0.779 mol, 5 mol %) and the reactor was purged again with nitrogen three times. The mixture was heated to 90-95° C. under N₂ protection and maintained at 90-95° C. for 2.5 h under N₂ protection at which point HPLC monitoring showed reaction completion.

The mixture was then cooled to 20-30° C. To this was charged EtOAc (15 kg) and this was maintained at 20-30° C. for 10 min. The resulting mixture was filtered through a pad of diatomite, washing the pad with EtOAc (15 kg×5). The filtrate was concentrated to dryness at 40-45° C., affording a black oil. This was dissolved in EtOAc/n-heptane (1:1 v/v, 26 L). To this was charged silica gel (3.3 kg) and this was maintained at room temperature for 30 min. The resulting mixture was filtered through a pad of silica gel (6.6 kg), washing the pad with EtOAc/n-heptane (1:1 v/v, 330 L). The filtrate was concentrated under reduced pressure at 40-45° C. (to ca. 7-8 kg of 5-(4,4,5,5-tetramethyl-1,3,2-dioxaborolan-2-yl)benzo[d]oxazol-2-amine (11′) as a suspension. To the suspension was charged MTBE (3.1 kg) and this was maintained at room temperature for 5 min. To the resulting mixture was charged n-heptane (5.7 kg) and this was maintained at room temperature for 30 mi. This was then cooled to 5-10° C. and maintained at 5-10° C. for 30 m. The resulting mixture was filtered, washing the caked with MTBE/n-heptane (1:5 v/v, 3.3L×2). The wet cake was dried under reduced pressure at 40-45° C. to constant weight affording 2.25 kg of 5-(4,4,5,5-tetramethyl-1,3,2-dioxaborolan-2-yl)benzo[d]oxazol-2-amine (11′) as a yellow solid (5600 uncorrected yield).

TABLE 9
HPLC Method for Part 9 of Example 1
HPLC method:

| Instrument: | Agilent 1260 series HPLC | |
|---|---|---|
| Column: | Waters XBridge C18 (3.0 × 150 mm, 3.5 μm) | |
| Mobile Phase: | A: 10 mM aq. NH₄OAc | |
| | B: MeCN | |
| | Time (min) | B (%) |
| Gradient: | 0.00 | 10 |
| | 2.00 | 10 |
| | 10.00 | 80 |
| | 11.00 | 90 |
| | 16.00 | 90 |

| | Post time: 5 min |
|---|---|
| Flow Rate: | 0.6 mL/min |
| UV Detector | 210 nm |
| Wavelength: | |
| Column Temperature: | 30° C. |
| Run Time: | 16.0 min |
| Retention Times: | 5-bromobenzo[d]oxazol-2-amine (11a′): 8.9 min |
| | 5-(4,4,5,5-tetramethyl-1,3,2-dioxaborolan-2- |
| | yl)benzo[d]oxazol-2-amine (11′): 9.9 min |

¹H NMR (400 MHz, CDCl₃) δ 7.80 (s, 1H), 7.57 (d, J=8 Hz, 1H), 7.27 (d, J=8 Hz, 1H), 5.55 (br s, 2H), 1.36 (s, 12H).

### Part 10-Synthesis of Compound 12′

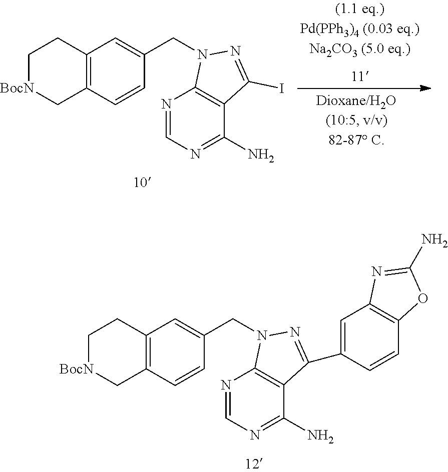 [SMILES: CC(C)(C)OC(=O)N1CCc2cc(Cn3nc(I)c4c(N)ncnc43)ccc2C1 ; CC(C)(C)OC(=O)N1CCc2cc(Cn3nc(-c4ccc5oc(N)nc5c4)c4c(N)ncnc43)ccc2C1]

### Compound 12′-tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate

To a 100-L reactor, with agitation, was charged tert-butyl 6-((4-amino-3-iodo-1H-pyrazolo[3,4-d]pyrimidin-1-yl)methyl)-3, 4-dihydroisoquinoline-2(1H)-carboxylate (10′ (3 kg, 5.92 mol, 1.0 eq.), 5-(4,4,5,5-tetramethyl-1,3,2-dioxaborolan-2-yl)benzo[d]oxazol-2-amine (11′) (1.85 kg, 6.52 mol, 1.1 eq. calculated based on its 92 wt % assay by qNMR), Na₂CO₃ (3.14 g, 29.6 mol, 5.0 eq.), dioxane (31 kg), and water (15 kg). The reactor was purged with nitrogen three times. To this was charged Pd(PPh₃)₄ (1.36 g, 1.18 mmol, 3 mol %) and the reactor was purged again with nitrogen three times. The mixture was heated to 82-87° C. and maintained at that temperature for 5 h at which point HPLC monitoring showed reaction completion.

The mixture was cooled to 15-25° C. and then charged into ice-water (75 kg) at below 10° C. and maintained at 0-10° C. for 30 min. This was then filtered, washing the cake with water (9 kg×2). The wet cake was slurried in MeOH (12 kg) at 65-70° C. for 30 min. This was allowed to cool down to 15-25° C. at a natural rate (required a period of 1.5 h) and then further cooled to 0-10° C. over a period of 30 min. The resulting mixture was filtered, washing the cake with chilled MeOH (4.8 kg and then 2.4 kg). The wet cake was dried 50-55° C. under reduced pressure to constant weight affording 2.6 kg of tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (12′) as a light brown solid (85% uncorrected yield).

TABLE 10
HPLC Method for Part 10 of Example 1
HPLC method:

| Instrument: | Agilent 1260 series HPLC | |
|---|---|---|
| Column: | Agilent Poroshell 120 EC-C18 (4.6 × 100 mm, 2.7 μm) | |
| Mobile Phase: | A: 0.1% aq. H₃PO₄ | |
| | B: MeCN | |
| | Time (min) | B (%) |
| Gradient: | 0.00 | 5 |
| | 7.00 | 90 |
| | 12.00 | 90 |

| | Post time: 3 min |
|---|---|
| Flow Rate: | 1.0 mL/min |
| UV Detector | 210 nm |
| Wavelength: | |
| Column | 30° C. |
| Temperature: | |
| Run Time: | 12.0 min |
| Retention Times: | tert-butyl 6-((4-amino-3-iodo-1H-pyrazolo[3,4- |
| | d]pyrimidin-1-yl)methyl)-3,4-dihydroisoquinoline- |
| | 2(1H)-carboxylate (10′): 6.7 min |
| | 5-(4,4,5,5-tetramethyl-1,3,2-dioxaborolan-2- |
| | yl)benzo[d]oxazol-2-amine (11′): 5.4 min |
| | tert-butyl 6-((4-amino-3-(2-aminobenzo[d]oxazol-5-yl)- |
| | 1H-pyrazolo[3,4-d]pyrimidin-1-yl)methyl)-3,4- |
| | dihydroisoquinoline-2(1H)-carboxylate (12′): 5.6 min |

¹H NMR (400 MHz, DMSO-d₆) δ 8.21 (s, 1H), 7.46-7.04 (m, 6H), 5.42 (br s, 2H), 4.36 (br s, 2H), 3.42 (br s, 2H), 2.64 (br s, 2H), 1.32 (s, 9H).

### Part 11-Synthesis of Compound 13b′

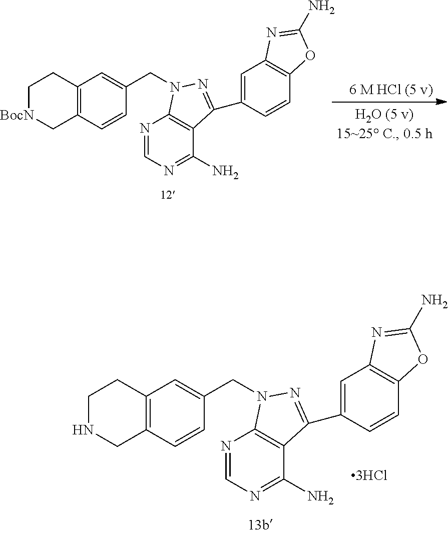 [SMILES: CC(C)(C)OC(=O)N1CCc2cc(Cn3nc(-c4ccc5oc(N)nc5c4)c4c(N)ncnc43)ccc2C1 ; Nc1nc2cc(-c3nn(Cc4ccc5c(c4)CCNC5)c4ncnc(N)c34)ccc2o1]

### Compound 13b′-5-(4-amino-1-((1,2,3,4-tetrahydroisoquinolin-6-yl)methyl)-1H-pyrazolo[3,4-d]pyrimidin-3-yl)benzo[d]oxazol-2-amine tris HCl

To a 50-L reactor, with agitation, was charged water (12 L). This was cooled to below 10° C. To this was charged 12 M aqueous HCl (12 L,144 mol, 30.7 eq.) slowly at below 20° C. To the reaction mixture was charged tert-butyl 6-((4-amino-3-(2-aminobenzo[d]oxazol-5-yl)-1H-pyrazolo[3,4-d]pyrimidin-1-yl)methyl)-3,4-dihydroisoquinoline-2(1H)-carboxylate (12′) (2.4 kg, 4.68 mol, 1.0 eq.) in 20 portions at 15-25° C. over a period of 120 min. This was maintained at 15-25° C. for 0.5 h at which point HPLC monitoring showed reaction completion.

The mixture was then heated to 40-50° C. To this was charged activated carbon (120 g, 5 w %) and the resulting mixture was maintained at 40-50° C. for 30 min. This was then filtered, washing the cake with warm water (40-50° C., 4.8L×2). To the filtrate was then charged i-PrOH (168 L) slowly over 60 min at 15-25° C. The resulting mixture was maintained at 15-25° C. for 30 min and then cooled to 5-10° C. and maintained at 5-10° C. for 30 min. The resulting mixture was then filtered, washing the cake with chilled i-PrOH (5-10° C., 7.2L×2) and then with n-heptane (7.2L×2). The wet cake was dried at 45-55° C. under reduced pressure to constant weight to afford 2.2 kg of 5-(4-amino-1-((1,2,3,4-tetrahydroisoquinolin-6-yl)methyl)-1H-pyrazolo[3,4-d]pyrimidin-3-yl)benzo[d]oxazol-2-amine tris HCl (13b′) as a red solid (99% corrected yield).

TABLE 11
HPLC Method for Part 11 of Example 1
HPLC method:

| Instrument: | Agilent 1260 series HPLC | |
|---|---|---|
| Column: | Waters XBridge C18 (3.0 × 150 mm, 3.5 μm) | |
| Mobile Phase: | A: 10 mM aq. NH₄OAc | |
| | B: MeCN | |
| | Time (min) | B (%) |
| Gradient: | 0.00 | 5 |
| | 2.00 | 5 |
| | 12.00 | 90 |
| | 20.00 | 90 |

| | Post time: 5 min |
|---|---|
| Flow Rate: | 0.6 mL/min |
| UV Detector | 210 nm |
| Wavelength: | |
| Column | 30° C. |
| Temperature: | |
| Run Time: | 20.0 min |
| Retention Times: | tert-butyl 6-((4-amino-3-(2-aminobenzo[d]oxazol-5-yl)- |
| | 1H-pyrazolo[3,4-d]pyrimidin-1-yl)methyl)-3,4- |
| | dihydroisoquinoline-2(1H)-carboxylate (12′): 10.7 min |
| | 5-(4-amino-1-((1,2,3,4-tetrahydroisoquinolin-6- |
| | yl)methyl)-1H-pyrazolo[3,4-d]pyrimidin-3- |
| | yl)benzo[d]oxazol-2-amine tris HCl (13b′): 7.1 min |

¹H NMR (400 MHz, D20) δ8.30 (s, 1H), 7.50 (d, J=8 Hz, 1H), 7.49 (s, 1H), 7.40 (d, J=8 Hz, 1H), 7.03-7.01 (m, 3H), 5.46 (s, 2H), 4.17 (s, 2H), 3.32 (t, J=6 Hz, 2H), 2.90 (t, J=6 Hz, 2H).

MS (ESI+): Calculated for C₂₂H₂₁N₈O (M+H+): 413.2. Found: 412.9.

### Example 2—Synthetic Protocol for Compound 25′

Detailed below is a general synthetic protocol for Compound 25′.

### Synthesis of Compound 25′-(1R,2R,4S)-2-methoxy-4-((R)-2-((3S,5R,6R,7E,9R,10R,12R,14S,15E,17E,19E,21S,23S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (4-nitrophenyl) carbonate

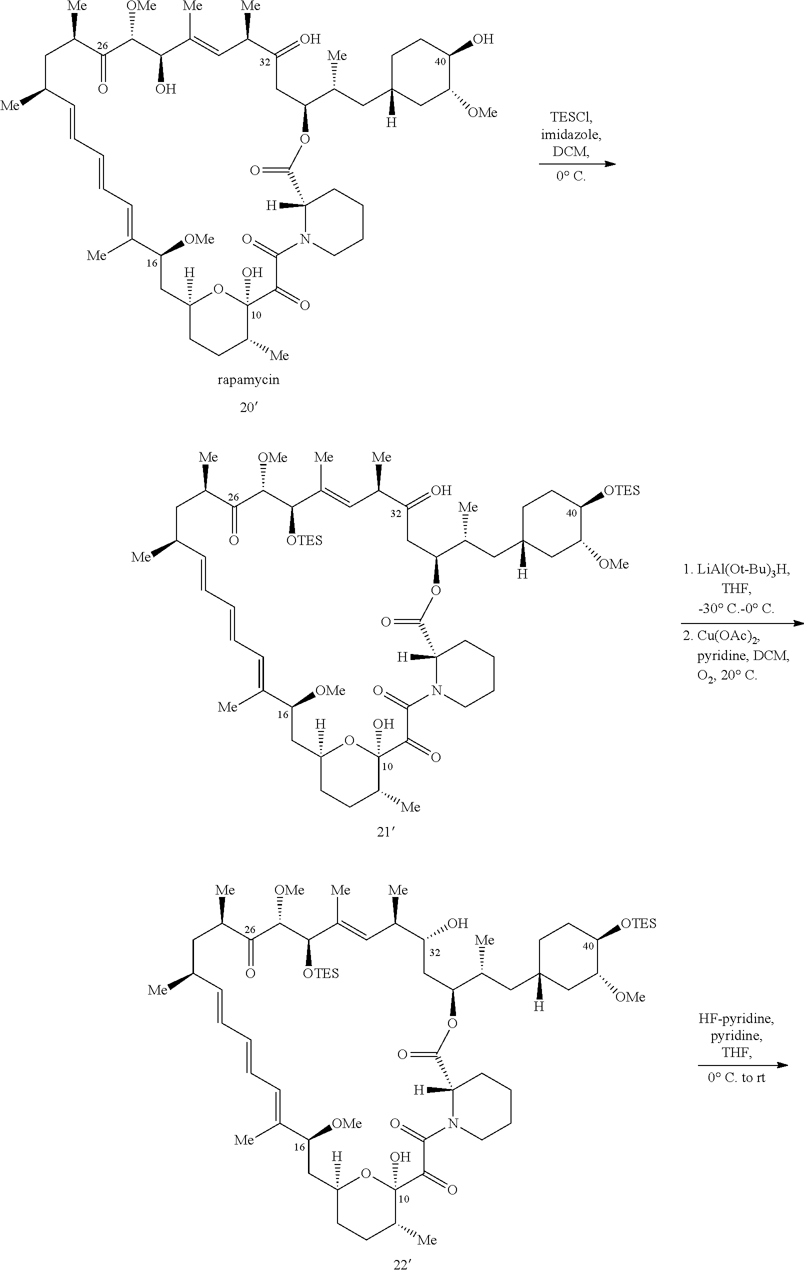

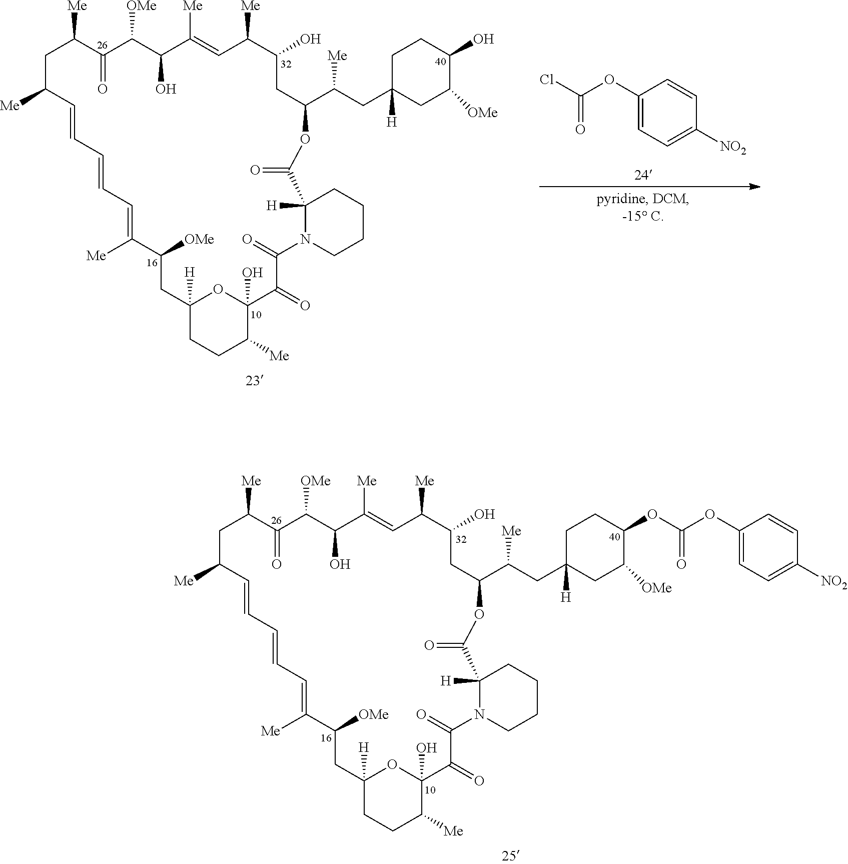 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; O=C(Cl)Oc1ccc([N+](=O)[O-])cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

Part 1-Synthesis of Compound 21

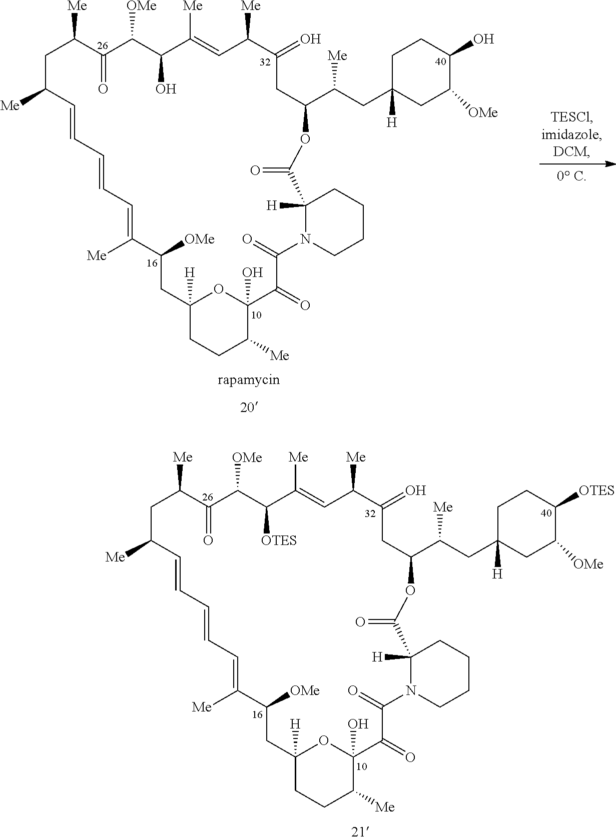

### Compound 21′-(3S,6R,7E,9R,10R,12R,14S,15E,17E,19E,21S,23S,26R,27R,34aS)-27-hydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone

To a 20-L flask, with agitation, was charged rapamycin (20′) (1100 g, 1.2 mol, 1 eq.) and DCM (10 L). The resulting solution was cooled 2° C. To this was charged imidazole (246 g, 3.6 mol, 3 eq.) in portions over 5 min. To this was charged TESCI (488 g, 3.2 mol, 2.7 eq.) over 1 h, maintaining temperature below 5° C. the resulting mixture was maintained at 0° C. for 4 h at which point HPLC monitoring showed reaction completion.

The mixture was filtered through a pad of Magnesol (400 g) into pre-chilled aqueous 0.5 M NaHCO₃/0.5 M NaCl (2 L), washing the cake with DCM (1 L). The organic phase was dried over 3 Å molecular sieves, filtered, and concentrated to afford (3S,6R,7E,9R,10R,12R,14S,15E,17E,19E,21S,23S,26R,27R,34aS)-27-hydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (21′) as a flaky yellow solid (1440 g, 85% w/w, 1.07 mol, 89% yield).

### Alternate Procedure

To a reactor, with agitation, was charged rapamycin (20′) (2.28 kg, 2.49 mol, 1 eq.) and DCM (30 kg). The resulting solution was cooled to and maintained at −5 to 5° C. To this was added imidazole (0.51 kg, 7.49 mol, 3 eq.) and TESCI (1.04 kg, 6.90 mol, 2.8 eq.). The resulting mixture was maintained at −5 to 5° C. for 2 hours at which point HPLC monitoring showed reaction completion.

The mixture was filtered through Florisil® (2.7 kg), washing the cake with DCM (5.7 kg). The filtrate was washed with aqueous 1 M NaCl/0.5 M NaHCO₃(11 kg). The organic phase was solvent exchanged to THE (total of 34 kg) and concentrated to afford (3S,6R,7E,9R,10R,12R,14S,15E,17E,19E,21S,23S,26R,27R,34aS)-27-hydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (21′) as a THF solution (9.4 kg, 26.6% w/w, 2.19 mol, 88% yield).

TABLE 12
HPLC Method for Part 1 of Example 2
HPLC method:

| Instrument: | Agilent 1260 Infinity II | | | |
|---|---|---|---|---|
| Column: | Eclipse XDB-C18 (4.6 × 50 mm, 1.8 μm) | | | |
| Mobile Phase: | A: water | | | |
| | B: MeCN | | | |
| | C: MeOH | | | |
| | Time (min) | A % | B % | C % |
| Gradient: | 0.0 | 24 | 38 | 38 |
| | 0.5 | 24 | 38 | 38 |
| | 2.0 | 10 | 45 | 45 |
| | 6.0 | 0 | 50 | 50 |
| | 10.0 | 0 | 50 | 50 |
| | 11.0 | 0 | 100 | 0 |
| | 31.0 | 0 | 100 | 0 |
| | 33.0 | 24 | 38 | 38 |
| | 35.0 | 24 | 38 | 38 |

| | |
|---|---|
| Flow Rate: | 1.3 mL/min |
| UV Detector | 280 nm |
| Wavelength: | |
| Column | 55° C. |
| Temperature: | |
| Retention | rapamycin (20′): 2.4 min |
| Times: | (3S,6R,7E,9R,10R,12R,14S,15E,17E,19E,21S,23S,26R, |
| | 27R,34aS)-27-hydroxy-10,21-dimethoxy-3-((R)-1- |
| | ((1S,3R,4R)-3-methoxy-4- |
| | ((triethylsilyl)oxy)cyclohexyl)propan-2-yl)- |
| | 6,8,12,14,20,26-hexamethyl-9-((triethylsilyl)oxy)- |
| | 9,10,12,13,14,21,22,23,24,25,26,27,32,33,34,34a- |
| | hexadecahydro-3H-23,27-epoxypyrido[2,1- |
| | c][1]oxa[4]azacyclohentriacontine- |
| | 1,5,11,28,29(4H,6H,31H)-pentaone (21′): 7.9 min |

Part 2-Synthesis of Compound 22′

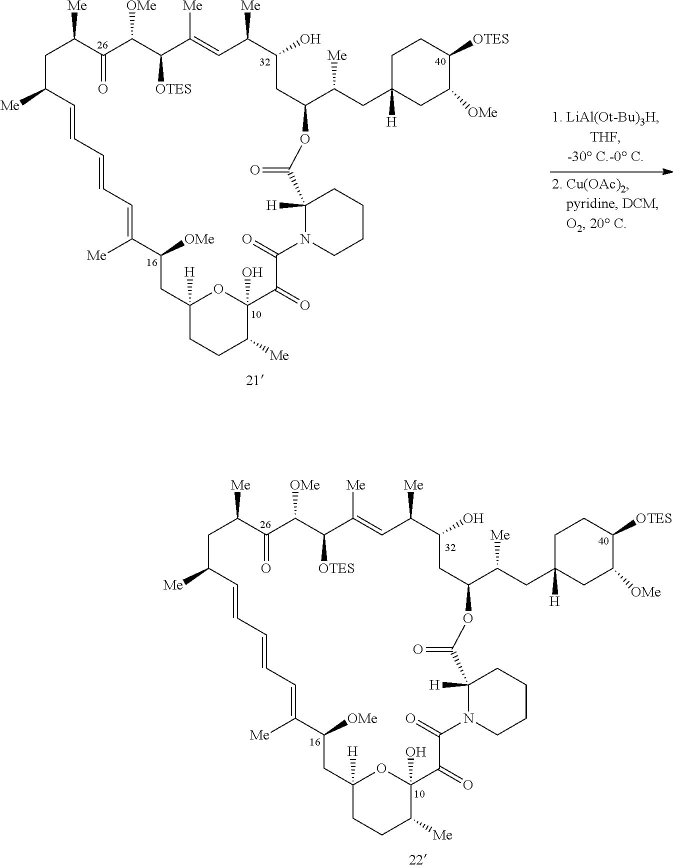

### Compound 22′-(3S,5R,6R,7E,9R,10R,12R,14S,15E,17E,19E,21S,23S,26R,27R,34aS)-5,27-dihydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone

To a 20-L flask, with agitation, was charged (3S,6R,7E,9R,10R,12R,14S,15E,17E,19E,21S,23S,26R,27R,34aS)-27-hydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (21′) (1.44 kg, 85% w/w, 1.07 mol, 1 eq.) and THF (10 L). This was cooled to −30° C. To this was charged LiAl(Ot-Bu)₃H (1.0 M in THF, 3.2 L, 3.2 mol, 3 eq.) over 1 h, maintaining temperature below −25° C. The resulting mixture was warmed to −10° C. over 3 h and then maintained at −10° C. overnight at which point HPLC monitoring showed reaction completion.

The mixture was diluted with pre-chilled (−20° C.) EtOAc (10 L). To this was charged aqueous 0.5 M citric acid/0.5 M NaCl (10 L). The organic phase was then washed with aqueous 0.5 M NaHCO₃/0.5 M NaCl (20 L), dried over 3 Å molecular sieves, filtered, and concentrated to dryness affording a white foam (1490 g).

To a 20-L flask, with agitation at room temperature, was charged the white foam and DCM (5 L). To this was charged pyridine (170 mL, 167 g, 2.1 mol, 2.0 eq.) and Cu(OAc)₂ (185 g, 1.02 mol, 0.95 eq.). Into the mixture was then bubbled ambient air for 1 h at which point HPLC monitoring showed reaction completion.

The mixture was filtered through a pad of Magnesol (400 g) and the filtrate concentrated to a green foam. This was purified in portions by silica gel column chromatography (EtOAc/heptane). The desired fractions were collected and concentrated to afford (3S,5R,6R,7E,9R,10R,12R,14S,15E,17E,19E,21S,23 S,26R,27R,34aS)-5,27-dihydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (22′) as a waxy solid (900 g, 87.6% w/w, 689 mmol, 64% yield).

### Alternate Procedure

To a reactor, with agitation, was charged a THF solution of (3S,6R,7E,9R,10R,12R,14S,15E,17E,19E,21S,23S,26R,27R,34aS)-27-hydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-9,10,12,13,14,21,22,23,24,25,26,27,32,33,34,34a-hexadecahydro-3H-23,27-epoxypyrido[2,1-c][1]oxa[4]azacyclohentriacontine-1,5,11,28,29(4H,6H,31H)-pentaone (21′) (8 kg, 26.6% w/w, 1.86 mol, 1 eq.) and THE (15 kg). This was cooled to and maintained at −35 to −25° C. To this was added LiAl(Ot-Bu)₃H (1.0 M in THF, 4.5 kg, 4.98 mol, 2.7 eq.). The resulting mixture was maintained for 2.5 hours and then warmed and maintained at −5 to 5° C. for 4 hours at which point HPLC monitoring showed reaction completion.

The mixture was quenched into a pre-cooled (−5 to 5° C.) mixture of 0.5 M aqueous citric acid (24 kg) and EtOAc (24 kg), rinsing the reactor forward with EtOAc (12 kg). The aqueous layer was extracted, maintaining −5 to 5° C., with EtOAc (12 kg). The combined organic layers were washed with aqueous 0.5 M NaHCO₃/1 M NaCl (21 kg), maintaining 0-10° C., and then dried over molecular sieves (0.6 kg), maintaining −5 to 5° C. This was then filtered, washing the cake with EtOAc (2.9 kg), solvent exchanged to DCM (total of 54 kg), and concentrated to approximately 6 volumes.

To this DCM solution, with agitation and maintained at 15-25° C., was added pyridine (0.41 kg, 5.18 mol, 2.8 eq.) and Cu(OAc)₂ (0.31 kg, 1.71 mol, 0.92 eq.). Into the mixture was then bubbled 5% oxygen for 5 h at which point HPLC monitoring showed reaction completion.

The reaction was then filtered, washing the cake with DCM (3 kg). The filtrate was washed with aqueous 0.5 M NaHCO₃/1 M NaCl (9.8 kg). The organic layer was filtered through Magnesol®, washing the cake with DCM (20 kg), and concentrated to approximately 5 volumes. This was combined with other batches and purified in portions by silica gel column chromatography (EtOAc/heptane). The desired fractions were collected and solvent exchanged to THE to afford (3S,5R,6R,7E,9R,10R,12R,14S,15E,17E,19E,21S,23 S,26R,27R,34aS)-5,27-dihydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (22′) as a THF solution (22.1 kg, 18.2% w/w, 3.51 mol, 48% yield).

TABLE 13
HPLC Method for Part 2 of Example 2
HPLC method:

| Instrument: | Agilent 1260 Infinity II | | | |
|---|---|---|---|---|
| Column: | Eclipse XDB-C18 (4.6 × 150 mm, 3.5 μm) | | | |
| Mobile Phase: | A: water | | | |
| | B: MeCN | | | |
| | C: MeOH | | | |
| | Time (min) | A % | B % | C % |
| Gradient: | 0 | 25 | 40 | 35 |
| | 1 | 25 | 40 | 35 |
| | 8 | 10 | 55 | 35 |
| | 24 | 0 | 65 | 35 |
| | 34 | 0 | 65 | 35 |
| | 35 | 0 | 100 | 0 |
| | 95 | 0 | 100 | 0 |
| | 97 | 25 | 40 | 35 |
| | 102 | 25 | 40 | 35 |

| | |
|---|---|
| Flow Rate: | 1.4 mL/min |
| UV Detector | 280 nm |
| Wavelength: | |
| Column | 55° C. |
| Temperature: | |
| Retention Times: | (3S,6R,7E,9R,10R,12R,14S,15E,17E,19E,21S,23S,26R, |
| | 27R,34aS)-27-hydroxy-10,21-dimethoxy-3-((R)-1- |
| | ((1S,3R,4R)-3-methoxy-4- |
| | ((triethylsilyl)oxy)cyclohexyl)propan-2-yl)- |
| | 6,8,12,14,20,26-hexamethyl-9-((triethylsilyl)oxy)- |
| | 9,10,12,13,14,21,22,23,24,25,26,27,32,33,34,34a- |
| | hexadecahydro-3H-23,27-epoxypyrido[2,1- |
| | c][1]oxa[4]azacyclohentriacontine- |
| | 1,5,11,28,29(4H,6H,31H)-pentaone (21′): 26.6 min |
| | (3S,5R,6R,7E,9R,10R,12R,14S,15E,17E,19E,21S,23S, |
| | 26R,27R,34aS)-5,27-dihydroxy-10,21-dimethoxy-3- |
| | ((R)-1-((1S,3R,4R)-3-methoxy-4- |
| | ((triethylsilyl)oxy)cyclohexyl)propan-2-yl)- |
| | 6,8,12,14,20,26-hexamethyl-9-((triethylsilyl)oxy)- |
| | 5,6,9,10,12,13,14,21,22,23,24,25,26,27,32,33,34,34a- |
| | octadecahydro-3H-23,27-epoxypyrido[2,1- |
| | c][1]oxa[4]azacyclohentriacontine-1,11,28,29(4H,31H)- |
| | tetraone (22′): 28.4 min |

Part 3-Synthesis of Compound 23′

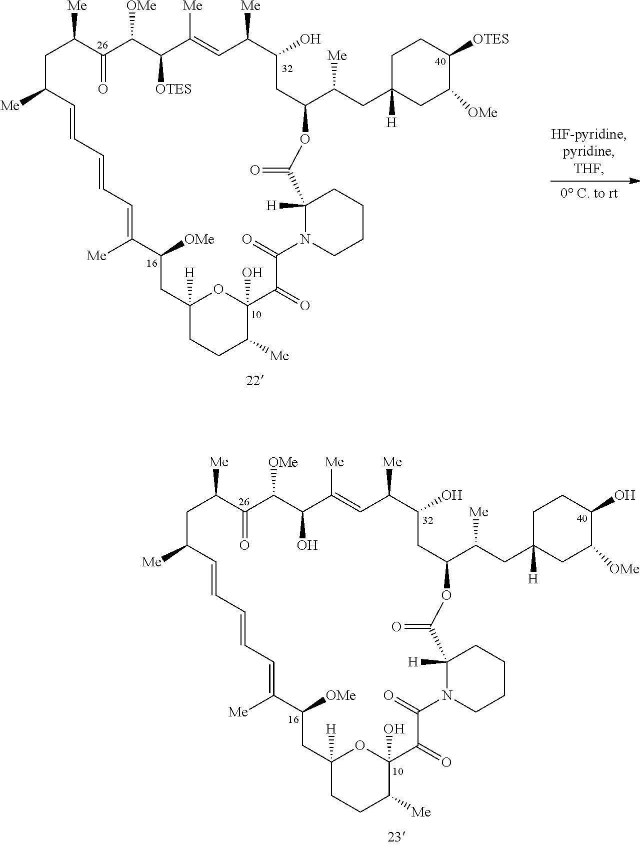

### Compound 23′-(3S,5R,6R,7E,9R,10R,12R,14S,15E,17E,19E,21S,23S,26R,27R,34aS)-5,9,27-trihydroxy-3-((R)-1-((1S,3R,4R)-4-hydroxy-3-methoxycyclohexyl)propan-2-yl)-PT27C 10,21-dimethoxy-6,8,12,14,20,26-hexamethyl-5,6,9,10,12,13,14,21,22,23,24,25,26,27,32,33,34,34a-octadecahydro-311-23,27-epoxypyrido12,1-cl 111oxa141azacyclohentriacontine-1,11,28,29(4H1,31H)-tetraone

To an 20-L HDPE container was charged (3 S,5R,6R,7E,9R,110R,12R,14S,15E,17E,19E,21S,23 S,26R,27R,34aS)-5,27-dihydroxy-10,21-dimethoxy-3-((R)-1-((1 S,3R,4R)-3-methoxy-4-((triethyl silyl)oxy)cyclohexyl)propan-2-yl)-6,8,12,14,20,26-hexamethyl-9-((tri ethyl silyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (22′) (900 g, 88% w/w, 692 mmol, 1 eq.) and pre-chilled (overnight in a freezer) THF (10 L). The resulting mixture was agitated, affording a solution of −10° C. internal temperature. To this was charged pre-chilled (overnight in a freezer) pyridine (2 L, 1.96 kg, 24.8 mol, 35.9 eq.). To this was then charged pre-chilled (overnight in a freezer) HF-pyridine (20% w/w HF in pyridine, 500 g, 5.0 mol, 7.2 eq.) in portions over 10 min affording a solution of 7° C. internal temperature. The mixture was maintained for 16 h, allowing the temperature to rise to ambient temperature at a natural rate at which point HPLC monitoring showed reaction completion.

The mixture was diluted with EtOAc (10 L) and washed with aqueous 0.5 M NaHCO₃/ saturated NaCl (10 L). The aqueous phase was extracted with EtOAc (1 L). The combined organic phases were dried over 3 Å molecular sieves, filtered, and concentrated to dryness. The resulting residue was dissolved in MTBE (1.5 L) and charged slowly, at room temperature, into heptane (14 L), forming a white precipitate. This was maintained at room temperature for 15 min. The mixture was filtered and the cake was dried in a vacuum chamber for 40 h, affording (3S,5R,6R,7E,9R,10R,12R,14S,15E,17E,19E,21S,23S,26R,27R,34aS)-5,9,27-trihydroxy-3-((R)-1-((1S,3R,4R)-4-hydroxy-3-methoxycyclohexyl)propan-2-yl)-10,21-dimethoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (23′) (680 g, 68.9% w/w, 511 mmol, 74% yield).

### Alternate Procedure

To a reactor, with agitation, was charged a THF solution of (3S,5R,6R,7E,9R,10R,12R,14S,15E,17E,19E,21S,23S,26R,27R,34aS)-5,27-dihydroxy-10,21-dimethoxy-3-((R)-1-((1S,3R,4R)-3-methoxy-4-((triethylsilyl)oxy)cyclohexyl)propan-2-yl)-6,8,12,14,20,26-hexamethyl-9-((triethylsilyl)oxy)-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (22′) as a THF solution (21.8 kg, 18.2% w/w, 3.47 mol, 1 eq.) and THE (13.5 kg). This was cooled to and maintained at −5 to 5° C. To this was added pyridine (10.95 kg, 138.43 mol, 39.9 eq.), HF-pyridine (4 kg), and THE (9 kg). This was warmed to and maintained at 15-25° C. for 4 h at which point HPLC monitoring showed reaction completion.

To the reaction was quenched into a mixture of NaHCO₃(6.4 kg), water (73.5 kg), and EtOAc (78 kg), maintaining 15-25° C. To this was then added THE (3.5 kg). The organic phase was washed with saturated aqueous NaCl (22 kg) and then concentrated to approximately 3 volumes. To this was charged MTBE (57 kg) and the resulting solution was concentrated to approximately 3 volumes and divided into three portions.

Each portion was added to n-heptane (31 kg) and the resulting mixture was maintained for 1 hour at which point it was cooled to 0-10° C. and maintained for 3 hours. This was filtered, washing the cake with n-heptane (1.5 kg).

Dried cakes multiple batches (total 1.734 kg) were combined and dissolved in IPAc (6.4 kg) at 10-20° C. To the resulting solution was added n-heptane (24.3 kg) which produced solids. This mixture was maintained for 12 hours and then filtered, washing the cake with n-heptane (4 kg). The cake was dried to afford (3S,5R,6R,7E,9R,10R,12R,14S,15E,17E,19E,21S,23S,26R,27R,34aS)-5,9,27-trihydroxy-3-((R)-1-((1S,3R,4R)-4-hydroxy-3-methoxycyclohexyl)propan-2-yl)-10,21-dimethoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (23′) as a crystalline solid (1.89 kg, 90.8% w/w, 1.87 mol, 81% yield).

TABLE 14
HPLC Method for Part 3 of Example 2
HPLC method:

| Instrument: | Agilent 1260 Infinity II | | | |
|---|---|---|---|---|
| Column: | Zorbax Eclipse XDB-C18 (4.6 × 50 mm, 1.8 μm) | | | |
| Mobile | A: water | | | |
| Phase: | B: MeCN | | | |
| | C: MeOH | | | |
| | Time (min) | A % | B % | C % |
| Gradient: | 0.0 | 30 | 35 | 35 |
| | 0.5 | 30 | 35 | 35 |
| | 5.5 | 25 | 40 | 35 |
| | 7.5 | 20 | 45 | 35 |
| | 7.6 | 0 | 65 | 35 |
| | 14.0 | 0 | 65 | 35 |
| | 15.0 | 0 | 100 | 0 |
| | 25.0 | 0 | 100 | 0 |
| | 26.0 | 30 | 35 | 35 |
| | 30.0 | 30 | 35 | 35 |

| | |
|---|---|
| Flow Rate: | 1.4 mL/min |
| UV Detector | 280 nm |
| Wavelength: | |
| Column | 55° C. |
| Temperature: | |
| Retention | (3S,5R,6R,7E,9R,10R,12R,14S,15E,17E,19E,21S,23S,26R, |
| Times: | 27R,34aS)-5,27-dihydroxy-10,21-dimethoxy-3-((R)- |
| | 1-((1S,3R,4R)-3-methoxy-4- |
| | ((triethylsilyl)oxy)cyclohexyl)propan-2-yl)- |
| | 6,8,12,14,20,26-hexamethyl-9-((triethylsilyl)oxy)- |
| | 5,6,9,10,12,13,14,21,22,23,24,25,26,27,32,33,34,34a- |
| | octadecahydro-3H-23,27-epoxypyrido[2,1- |
| | c][1]oxa[4]azacyclohentriacontine-1,11,28,29(4H,31H)- |
| | tetraone (22′): 11.8 min |
| | ((3S,5R,6R,7E,9R,10R,12R,14S,15E,17E,19E,21S,23S, |
| | 26R,27R,34aS)-5,9,27-trihydroxy-3-((R)-1- |
| | ((1S,3R,4R)-4-hydroxy-3-methoxycyclohexyl)propan- |
| | 2-yl)-10,21-dimethoxy-6,8,12,14,20,26-hexamethyl- |
| | 5,6,9,10,12,13,14,21,22,23,24,25,26,27,32,33,34,34a- |
| | octadecahydro-3H-23,27-epoxypyrido[2,1- |
| | c][1]oxa[4]azacyclohentriacontine-1,11,28,29(4H,31H)- |
| | tetraone (23′): 4.5 min |

Part 4-Synthesis of Compound 25′

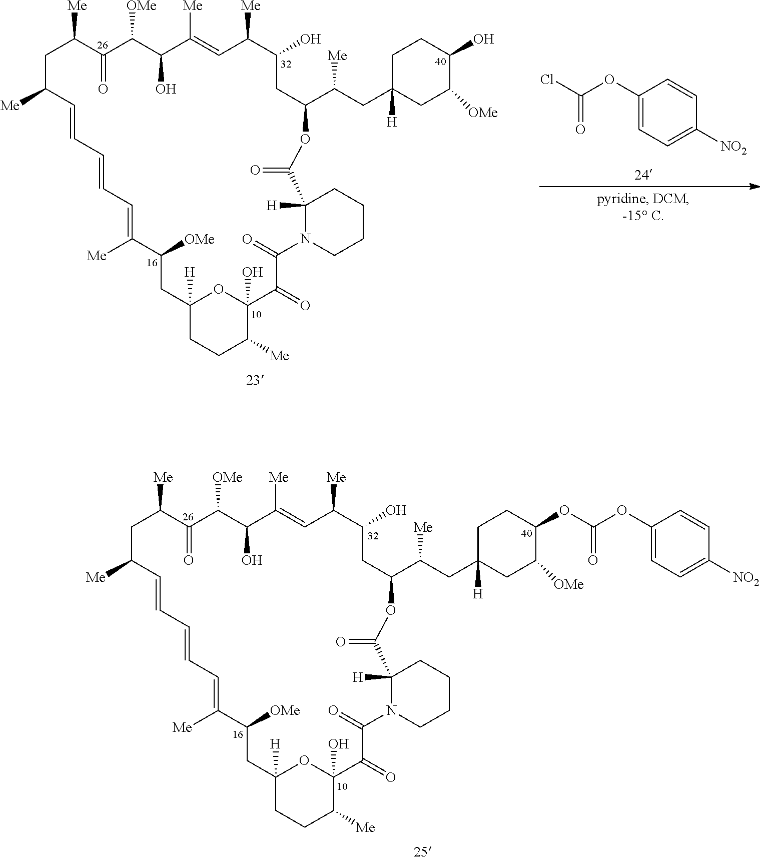 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C ; O=C(Cl)Oc1ccc([N+](=O)[O-])cc1 ; CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]

### Compound 25′-(1R,2R,4S)-2-methoxy-4-((R)-2-((3S,5R,6R,7E,9R,10R,12R,14S,15E,17E,19E,21S,23S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-311-23,27-epoxypyrido12,1-cl[11oxa141azacyclohentriacontin-3-yl)propyl)cyclohexyl (4-nitrophenyl) carbonate

To a 10-L flask, with agitation, was charged 3 Å powdered molecular sieves and DCM (5 L). The resulting mixture was cooled to −15° C. and maintained overnight. To this was then charged (3 S,5R,6R,7E,9R,10R,12R,14S,1SE,17E,19E,21 S,23 S,26R,27R,34aS)-5, 9,27-trihydroxy-3-((R)-1-((1 S,3R,4R)-4-hydroxy-3-methoxycyclohexyl)propan-2-yl)-10,21-dimethoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (23′) (665 g, 68.90% w/w, 500 mmol, 1 eq.) and the resulting mixture was maintained for 30 min. To this was then charged pyridine (0.6 L, 589 g, 7.45 mol, 14.9 eq.) and p-nitrophenylchloroformate (24′) (140 g, 695 mmol, 1.4 eq.). The resulting mixture was maintained at −15° C. for 5 h at which point HPLC monitoring showed reaction completion.

The mixture was filtered through a pad of Magnesol (400 g) and the filtrate was partially concentrated (to ca. 1.5 L). This concentrated was purified in portions by silica gel column chromatography (EtOAc/heptane). The desired fractions were collected and concentrated to afford (1R,2R,4S)-2-methoxy-4-((R)-2((3S,5R,6R,7E,9R,10R,12R,14S,15E,17E,19E,21S,23S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (4-nitrophenyl) carbonate (25′) (315 g, 87.6% w/w, 255 mmol, 51% yield).

### Alternate Procedure

To a reactor, with agitation, was charged

(3S,5R,6R,7E,9R,10R,12R,14S,15E,17E,19E,21S,23S,26R,27R,34aS)-5,9,27-trihydroxy-3-((R)-1-((1S,3R,4R)-4-hydroxy-3-methoxycyclohexyl)propan-2-yl)-10,21-dimethoxy-6,8,12,14,20,26-hexamethyl-5,6,9,10,12,13,14,21,22,23,24,25,26,27,32,33,34,34a-octadecahydro-3H-23,27-epoxypyrido[2,1-c][1]oxa[4]azacyclohentriacontine-1,11,28,29(4H,31H)-tetraone (23′) (net 852.1 g, 930.0 mmol, 1 eq.), molecular sieves (0.9 kg), and DCM (14.96 kg). The resulting mixture was maintained at 15-25° C. for 3 hours and then cooled to and maintained at −20 to −10° C. To this was added pyridine (0.957 kg, 12.10 mol, 13 eq.) and p-nitrophenylchloroformate (24′) (0.226 kg, 1.12 mol, 1.2 eq.). The resulting mixture was maintained at −20 to −10° C. for 9 hours at which point more p-nitrophenylchloroformate (24′) was added (38 g, total 0.264 kg, total 1.31 mol, total 1.4 eq.). This was maintained for 5 hours at which point more p-nitrophenylchloroformate (24′) was added (25 g, total 0.289 kg, total 1.43 mol, total 1.5 eq.). This was maintained for 5 hours at which point HPLC monitoring showed reaction completion.

The mixture was filtered through Magnesol® (0.85 kg), washing the cake with DCM (4 kg). The filtrate was added to aqueous 6% w/w NaCl/4% w/w NaHCO₃(9.0 kg) and maintained at 0-10° C. for 2 hours. The organic layer was washed with aqueous 6% w/w NaCl/4% w/w NaHCO₃(8.9 kg) (maintained at 0-10° C. for 2 hours), dried over molecular sieves (0.6 kg), and filtered, washing the cake with DCM (4 kg). The filtrate was concentrated to approximately 3 volumes.

Filtrates from multiple batches were combined and purified in portions by reverse phase preparative-HPLC (acetonitrile/water). The desired fraction were collected and lyophilized to afford (1R,2R,4S)-2-methoxy-4-((R)-2((3 S,5R,6R,7E,9R,110R,12R,14S,15E,17E,19E,21 S,23 S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (4-nitrophenyl) carbonate (25′) as a light yellow solid (1.16 kg, 90.5% w/w, 969.9 mmol).

TABLE 15
HPLC Method for Part 4 of Example 2
HPLC method:

| Instrument: | Agilent 1260 Infinity II | | | |
|---|---|---|---|---|
| Column: | Zorbax Eclipse XDB-C18 (4.6 × 50 mm, 1.8 μm) | | | |
| Mobile Phase: | A: water | | | |
| | B: MeCN | | | |
| | C: MeOH | | | |
| | Time (min) | A % | B % | C % |
| Gradient: | 0.0 | 25 | 35 | 40 |
| | 10.0 | 10 | 50 | 40 |
| | 10.1 | 5 | 55 | 40 |
| | 12.0 | 0 | 60 | 40 |
| | 12.1 | 0 | 100 | 0 |
| | 27.1 | 0 | 100 | 0 |
| | 27.2 | 25 | 35 | 40 |
| | 30.0 | 25 | 35 | 40 |

| | |
|---|---|
| Flow Rate: | 1.1 mL/min |
| UV Detector | 280 nm |
| Wavelength: | |
| Column | 55° C. |
| Temperature: | |
| Retention | (3S,5R,6R,7E,9R,10R,12R,14S,15E,17E,19E,21S,23S, |
| Times: | 26R,27R,34aS)-5,9,27-trihydroxy-3-((R)-1-((1S,3R,4R)- |
| | 4-hydroxy-3-methoxycyclohexyl)propan-2-yl)-10,21- |
| | dimethoxy-6,8,12,14,20,26-hexamethyl- |
| | 5,6,9,10,12,13,14,21,22,23,24,25,26,27,32,33,34,34a- |
| | octadecahydro-3H-23,27-epoxypyrido[2,1- |
| | c][1]oxa[4]azacyclohentriacontine-1,11,28,29(4H,31H)- |
| | tetraone (23′): 3.1 min |
| | (1R,2R,4S)-2-methoxy-4-((R)-2- |
| | ((3S,5R,6R,7E,9R,10R,12R,14S,15E,17E,19E,21S,23S, |
| | 26R,27R,34aS)-5,9,27-trihydroxy-10,21-dimethoxy- |
| | 6,8,12,14,20,26-hexamethyl-1,11,28,29-tetraoxo- |
| | 1,4,5,6,9,10,11,12,13,14,21,22,23,24,25,26,27,28,29,31, |
| | 32,33,34,34a-tetracosahydro-3H-23,27- |
| | epoxypyrido[2,1-c][1]oxa[4]azacyclohentriacontin-3- |
| | yl)propyl)cyclohexyl (4-nitrophenyl) carbonate (25′): |
| | 6.6 min |

### Example 3—Synthetic Protocol for Compound 33′

Detailed below is a general synthetic protocol for Compound 33′

### Synthesis of Compound 33′-(1R,2R,4S)-2-methoxy-4-((R)-2-((3S,5R,6R,7E,9R,10R,12R,14S,15E,17E,19E,21S,235,26R,27R,34a5)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1][1]oxa[4]azacyclohentriacontin-3-yl)propyl)cyclohexyl (27-(6-((4-amino-3-(2-aminobenzo[d]oxazol-5-yl)-lil-pyrazolo[3,4-d]pyrimidin-1-yl)methyl)-3,4-dihydroisoquinolin-2(1H)-yl)-27-oxo-3,6,9,12,15,18,21,24-octaoxaheptacosyl)carbamate

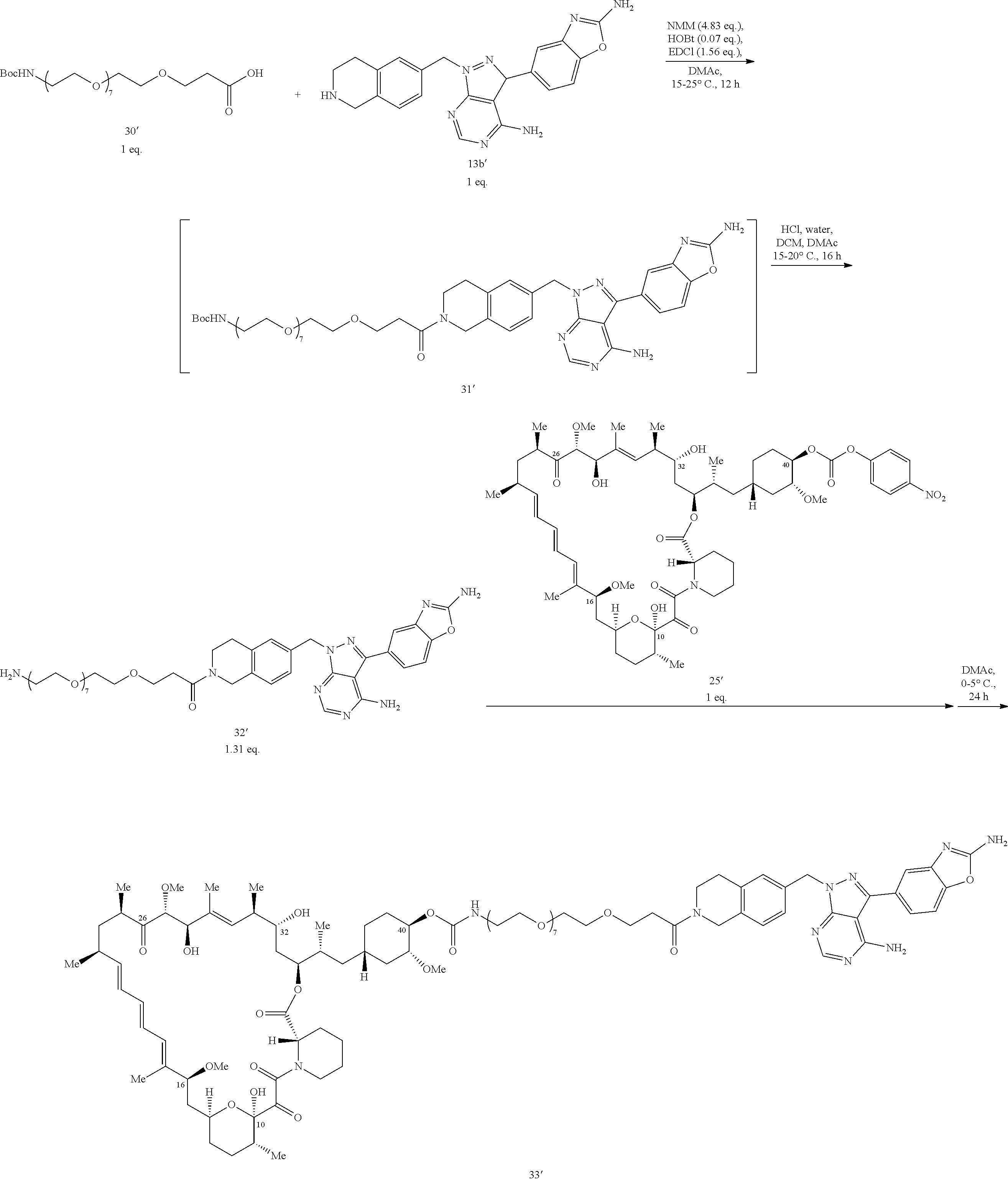

### Part 1-Synthesis of Compound 31′

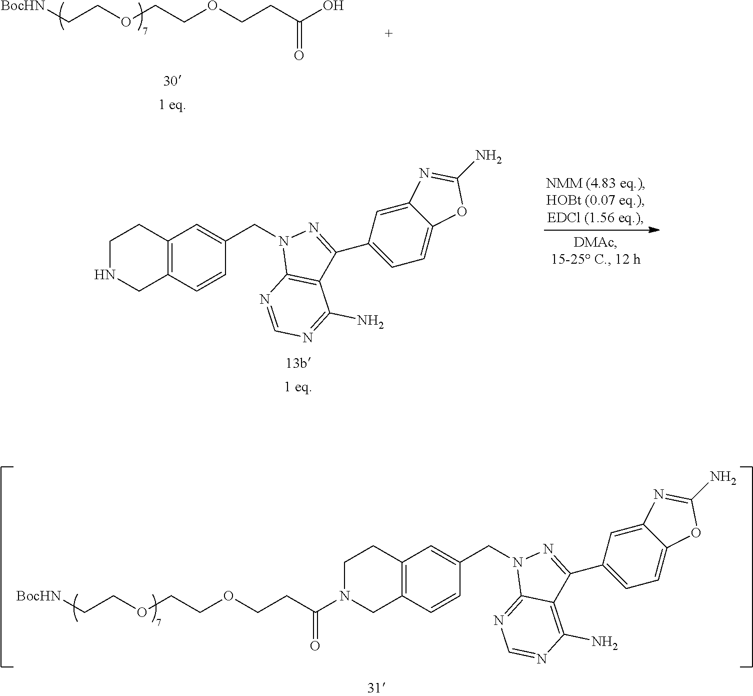

### Compound 31′-tert-butyl (27-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-27-oxo-3,6,9,12,15,18,21,24-octaoxaheptacosyl)carbamate

To a 50-L reactor, with agitation and under nitrogen protection, was charged 2,2-dimethyl-4-oxo-3,8,11,14,17,20,23,26,29-nonaoxa-5-azadotriacontan-32-oic acid (30′) (0.92 kg, 1.70 mol, 1 eq.), 5-(4-amino-1-((1,2,3,4-tetrahydroisoquinolin-6-yl)methyl)-1H-pyrazolo[3,4-d]pyrimidin-3-yl)benzo[d]oxazol-2-amine tris HCl (13b′) (0.928 kg, 1.70 mol (assay corrected), 1 eq.), NN (0.83 kg, 8.21 mol, 4.83 eq.), and DMAc (5.77 kg). This mixture was maintained at 15-25° C. for 30 min. To the reaction mixture was then charged HOBt (16.07 g, 0.12 mol, 0.07 eq.), EDCI (0.51 kg, 2.66 mol, 1.56 eq.) and DMAc (0.80 kg). The resulting mixture was maintained at 15-25° C. for 12 h at which point HPLC monitoring showed reaction completion.

The mixture was then diluted with DCM (58.4 kg) and washed with 13% w/w aqueous NaCl (100 kg×2). The organic phase was partially concentrated under reduced pressure at below 30° C. (to ca. 17 L). This mixture was then diluted with DCM (10 kg) to afford a DCM/DMAc solution of tert-butyl (27-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-27-oxo-3,6,9,12,15,18,21,24-octaoxaheptacosyl)carbamate (31′). This solution was used directly in the next step.

TABLE 16
HPLC Method for Part 1 of Example 3
HPLC method:

| Column | Agilent XDB C18 (4.6 × 50 mm, 1.8 μm) | | |
|---|---|---|---|
| Wavelength | 220 nm | | |
| Column | 25° C. | | |
| Temperature | | | |
| Auto-Samper | 5° C. | | |
| Temperature | | | |
| Flow Rate | 1.0 mL/min | | |
| Mobile Phase A | 0.05% TFA in water, v/v | | |
| Mobile Phase B | 0.05% TFA in ACN, v/v | | |
| | Time (min) | A % | B % |
| Gradient | Initial | 90 | 10 |
| Program | 1.00 | 90 | 10 |
| | 4.50 | 80 | 20 |
| | 8.50 | 60 | 40 |
| | 11.50 | 0 | 100 |
| | 13.50 | 0 | 100 |
| | 13.60 | 90 | 10 |
| | 20.00 | 90 | 10 |

| | |
|---|---|
| Run Time | 20 min |
| Retention | EDCI: 0.6 min |
| Times | DMAc: 0.8 min |
| | HOBt: 1.6 min |
| | 5-(4-amino-1-((1,2,3,4-tetrahydroisoquinolin- |
| | 6-yl)methyl)-1H-pyrazolo[3,4-d]pyrimidin- |
| | 3-yl)benzo[d]oxazol-2-amine tris HCl |
| | (13b′): 4.2 min |
| | tert-butyl (27-(6-((4-amino-3-(2-aminobenzo[d] |
| | oxazol-5-yl)-1H-pyrazolo[3,4-d]pyrimidin-1- |
| | yl)methyl)-3,4-dihydroisoquinolin-2(1H)-yl)- |
| | 27-oxo-3,6,9,12,15,18,21,24- |
| | octaoxaheptacosyl)carbamate (31′): 8.8 min |

### Part 2-Synthesis of Compound 32′

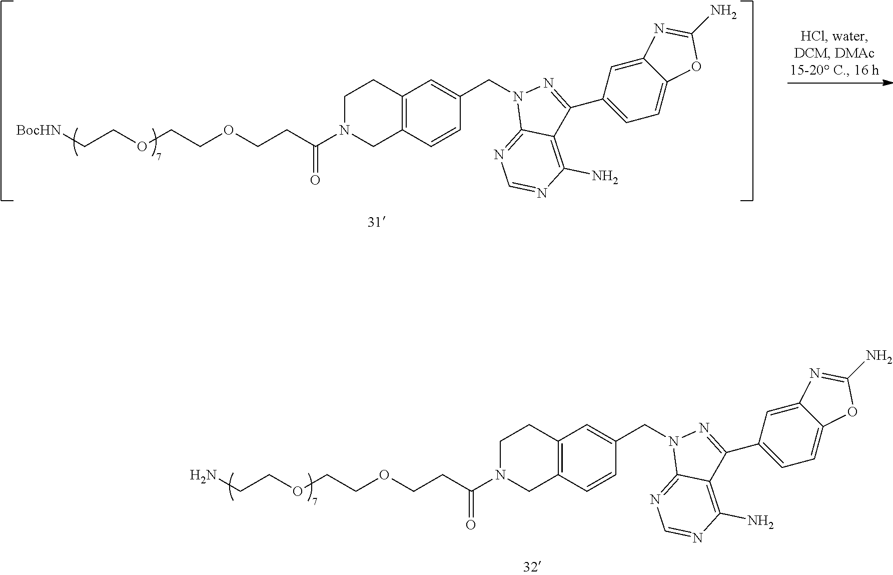

### Compound 32′-1-amino-27-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-3,6,9,12,15,18,21,24-octaoxaheptacosan-27-one

To a 50-reactor, with agitation and under nitrogen protection, was charged the DCM/DMAc solution of tert-butyl (27-(6-((4-amino-3-(2-aminobenzo[d]oxazol-5-yl)-1H-pyrazolo[3,4-d]pyrimidin-1l-yl)methyl)-3,4-dihydroisoquinolin-2(1H)-yl)-27-oxo-3,6,9,12,15,18,21,24-octaoxaheptacosyl)carbamate (31′) (ca. 1.70 mol) from the previous step. This was partially concentrated under reduced pressure at below 30° C. (to ca. 2 L). To this was then charged water (5.8 kg) and the resulting mixture was cooled to 0-5° C. To this was then charged 35% aqueous HCl (2.7 kg) at 0-15° C. The resulting mixture was maintained at 15-20° C. for 16 h at which point HPLC monitoring showed reaction completion.

The mixture was then diluted with DCM (63.5 kg) and cooled to 0-5° C. To this was then charged 3000 aqueous NaOH at 0-10° C. until pH of 11-12 (3.4 kg). This was then washed with 130w/w aqueous NaCl (100 kg×1). The aqueous phase was extracted with DCM (45 kg×2) and the combined organic phases were partially concentrated under reduced pressure at below 30° C. (to ca. 2 L). This afforded a 3.40 kg of a DCM/DMAc solution of 1-amino-27-(6-((4-amino-3-(2-aminobenzo[d]oxazol-5-yl)-H-pyrazolo[3,4-d]pyrimidin--yl)methyl)-3,4-dihydroisoquinolin-2(1H)-yl)-3,6,9,12,15,18,21,24-octaoxaheptacosan-27-one (32′) (35.7% w/w assay, 1.45 mol, 850 yield from 5-(4-amino-1-((1,2,3,4-tetrahydroisoquinolin-6-yl)methyl)-1H-pyrazolo[3,4-d]pyrimidin-3-yl)benzo[d]oxazol-2-amine tris HCl (13b′)).

TABLE 17
HPLC Method for Part 2 of Example 3
HPLC method:

| Column | Agilent XDB C18 (4.6 × 50 mm, 1.8 μm) | | |
|---|---|---|---|
| Wavelength | 220 nm | | |
| Column Temperature | 25° C. | | |
| Auto-Samper | 5° C. | | |
| Temperature | | | |
| Flow Rate | 1.0 mL/min | | |
| Mobile Phase A | 0.05% TFA in water, v/v | | |
| Mobile Phase B | 0.05% TFA in ACN, v/v | | |
| | Time (min) | A % | B % |
| Gradient Program | Initial | 90 | 10 |
| | 1.00 | 90 | 10 |
| | 4.50 | 80 | 20 |
| | 8.50 | 60 | 40 |
| | 11.50 | 0 | 100 |
| | 13.50 | 0 | 100 |
| | 13.60 | 90 | 10 |
| | 20.00 | 90 | 10 |

| | |
|---|---|
| Run Time | 20 min |
| Retention Times | DMAc: 0.8 min |
| | 1-amino-27-(6-((4-amino-3-(2-aminobenzo[d]oxazol-5-yl)-1H- |
| | pyrazolo[3,4-d]pyrimidin-1-yl)methyl)-3,4-dihydroisoquinolin- |
| | 2(1H)-yl)-3,6,9,12,15,18,21,24-octaoxaheptacosan-27-one (32′): |
| | 7.0 min |
| | tert-butyl (27-(6-((4-amino-3-(2-aminobenzo[d]oxazol-5-yl)-1H- |
| | pyrazolo[3,4-d]pyrimidin-1-yl)methyl)-3,4-dihydroisoquinolin- |
| | 2(1H)-yl)-27-oxo-3,6,9,12,15,18,21,24- |
| | octaoxaheptacosyl)carbamate (31′): 8.9 min |

### Part 3-Synthesis of Compound 33′

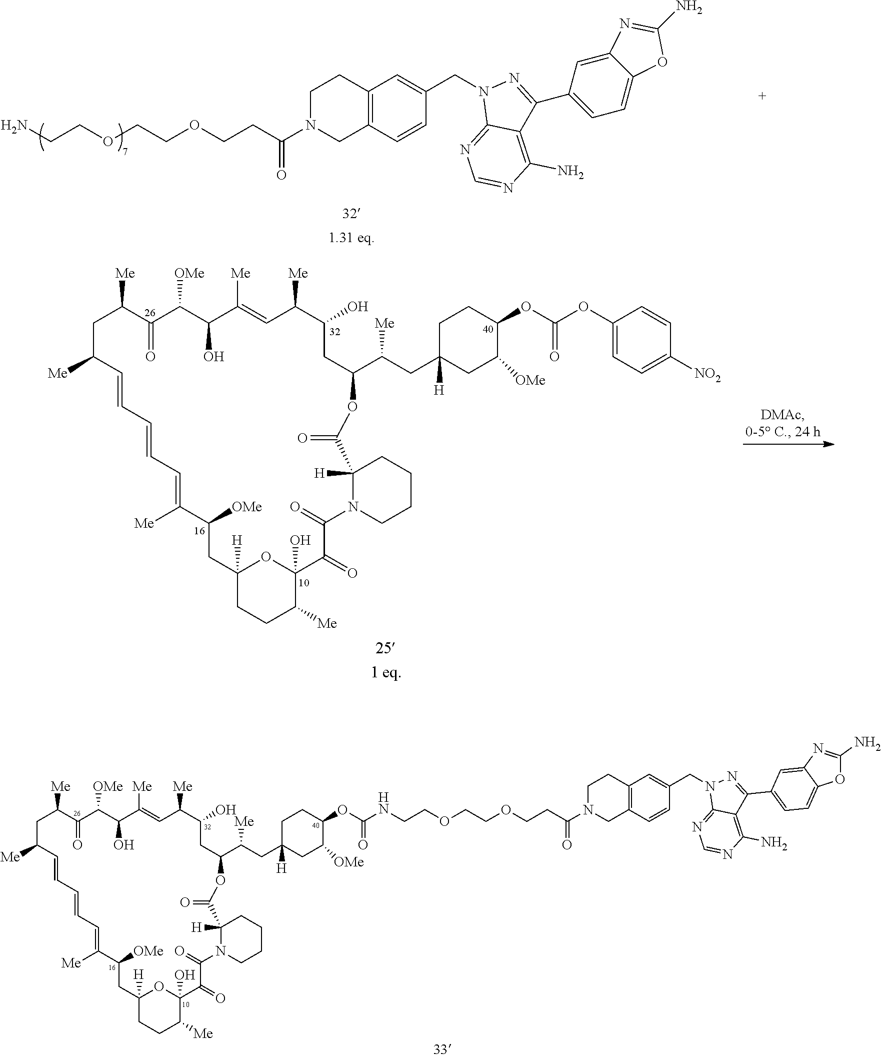

### Compound 33′-(1R,2R,4S)-2-methoxy-4-((R)-2-((3S,5R,6R,7E,9R,10R,12R,14S,15E,17E,19E,21S,23S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (27-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-27-oxo-3,6,9,12,15,18,21,24-octaoxaheptacosyl)carbamate

To a reactor, with agitation and under nitrogen protection, was charged a DCM/DMAc solution of 1-amino-27-(6-((4-amino-3-(2-aminobenzo[d]oxazol-5-yl)-1H-pyrazolo[3,4-d]pyrimidin-1-yl)methyl)-3,4-dihydroisoquinolin-2(1H)-yl)-3,6,9,12,15,18,21,24-octaoxaheptacosan-27-one (32′) (1.568 kg, 35.7% w/w assay, 0.67 mol, 1.31 eq.) and DMAc (0.89 kg). The resulting mixture was cooled to 0-5° C. To this was charged (1R,2R,4S)-2-methoxy-4-((R)-2-((3S,5R,6R,7E,9R,10R,12R,14S,15E,17E,19E,21S,23 S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (4-nitrophenyl) carbonate (25′) (0.554 kg, 0.51 mol, 1 eq.) and DMAc (0.31 kg). The resulting mixture was maintained at 0-5° C. for 24 h at which point HPLC monitoring showed reaction completion.

The mixture was diluted with DMAc (0.3 kg). This afforded 3.128 kg of a DCM/DMAc solution of (1R,2R,4S)-2-methoxy-4-((R)-2-((3S,5R,6R,7E,9R,10R,12R,14S,15E,17E,19E,21S,23S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (27-(6-((4-amino-3-(2-aminobenzo[d]oxazol-5-yl)-1H-pyrazolo[3,4-d]pyrimidin-1l-yl)methyl)-3,4-dihydroisoquinolin-2(1H)-yl)-27-oxo-3,6,9,12,15,18,21,24-octaoxaheptacosyl)carbamate (33′) (27.0Rw/w assay, 0.47 mol, 950 crude yield).

M004141 The crude solution was then purified by prep-(PLC (MeCN, water, formic acid) to afford (1R,2R,4S)-2-methoxy-4-((R)-2-((3 S,5R,6R,7E,9R,110R,12R,14S,15E,17E,19E,21 S,23 S,26R,27R,34aS)-5,9,27-trihydroxy-10,21-dimethoxy-6,8,12,14,20,26-hexamethyl-1,11,28,29-tetraoxo-1,4,5,6,9,10,11,12,13,14,21,22,23,24,25,26,27,28,29,31,32,33,34,34a-tetracosahydro-3H-23,27-epoxypyrido[2,1-c][1]oxa[4]azacyclohentriacontin-3-yl)propyl)cyclohexyl (27-(6-((4-amino-3-(2-aminobenzo[d]oxazol-5-yl)-1H-pyrazolo[3,4-d]pyrimidin-1l-yl)methyl)-3,4-dihydroisoquinolin-2(1H)-yl)-27-oxo-3,6,9,12,15,18,21,24-octaoxaheptacosyl)carbamate (33′) as a colorless amorphous solid (417.16 g, 0.23 mol, 450 yield).

TABLE 18
HPLC Method for Part 3 of Example 3
HPLC method:

| Column | RRHD Eclipse XDB-C18 (3.0 × 150 mm, 1.8 μm) | | |
|---|---|---|---|
| Wavelength | 280 nm | | |
| Column | 57° C. | | |
| Temperature | | | |
| Autosampler | 5° C. | | |
| Temperature | | | |
| Flow Rate | 0.7 mL/min | | |
| Mobile Phase A | 0.05% formic acid (FA) in water, v/v | | |
| Mobile Phase B | 0.05% formic acid (FA) in ACN, v/v | | |
| | Time (min) | A % | B % |
| Gradient | 0.00 | 80 | 20 |
| Program | 0.50 | 80 | 20 |
| | 3.50 | 70 | 30 |
| | 4.00 | 40 | 60 |
| | 8.50 | 35 | 65 |
| | 15.50 | 25 | 75 |
| | 18.50 | 0 | 100 |
| | 21.50 | 0 | 100 |
| | 21.60 | 80 | 20 |
| | 26.00 | 80 | 20 |

| | |
|---|---|
| Run Time | 26 min |
| Retention Times | 1-amino-27-(6-((4-amino-3-(2-aminobenzo[d]oxazol-5-yl)-1H- |
| | pyrazolo[3,4-d]pyrimidin-1-yl)methyl)-3,4-dihydroisoquinolin-2(1H)- |
| | yl)-3,6,9,12,15,18,21,24-octaoxaheptacosan-27-one (32′): 3.0 min |
| | (1R,2R,4S)-2-methoxy-4-((R)-2- |
| | ((3S,5R,6R,7E,9R,10R,12R,14S,15E,17E,19E,21S,23S,26R,27R,34aS)- |
| | 5,9,27-trihydroxy-10,21-dimethoxy-6,8,12,14,20,26-hexamethyl- |
| | 1,11,28,29-tetraoxo- |
| | 1,4,5,6,9,10,11,12,13,14,21,22,23,24,25,26,27,28,29,31,32,33,34,34a- |
| | tetracosahydro-3H-23,27-epoxypyrido[2,1- |
| | c][1]oxa[4]azacyclohentriacontin-3-yl)propyl)cyclohexyl (27-(6-((4- |
| | amino-3-(2-aminobenzo[d]oxazol-5-yl)-1H-pyrazolo[3,4-d]pyrimidin- |
| | 1-yl)methyl)-3,4-dihydroisoquinolin-2(1H)-yl)-27-oxo- |
| | 3,6,9,12,15,18,21,24-octaoxaheptacosyl)carbamate (33′): 8.8 min |
| | (1R,2R,4S)-2-methoxy-4-((R)-2- |
| | ((3S,5R,6R,7E,9R,10R,12R,14S,15E,17E,19E,21S,23S,26R,27R,34aS)- |
| | 5,9,27-trihydroxy-10,21-dimethoxy-6,8,12,14,20,26-hexamethyl- |
| | 1,11,28,29-tetraoxo- |
| | 1,4,5,6,9,10,11,12,13,14,21,22,23,24,25,26,27,28,29,31,32,33,34,34a- |
| | tetracosahydro-3H-23,27-epoxypyrido[2,1- |
| | c][1]oxa[4]azacyclohentriacontin-3-yl)propyl)cyclohexyl (4- |
| | nitrophenyl) carbonate (25′): 18.1 min |

HIRMS (ESI+). Calculated for C₉₃H₁₃₆N₁₀O₂₄Na (M+Na⁺): 1799.96212. Found: 1799.96338

Elemental Analysis

| | % C | % H | % N |
|---|---|---|---|
| Calculated | 62.82 | 7.71 | 7.88 |
| Found | 63.34 | 7.59 | 8.04 |

¹H NMR (700 MHz, CDCl₃) δ8.37 (m, 1H), 7.58 (br s, 1H), 7.35 (o, 1H), 7.34 (o, 1H), 7.23 (d, J=7.80 Hz) and 7.21 (d, J=7.84 Hz) (1H), 7.15 (br s) and 7.17 (br s) (1H), 7.07 (d, J=8.05 Hz) and 7.02 (d, J=8.05 Hz) (1H), 6.36 (o, 1H), 6.32 (o, 1H), 6.12 (o, 1H), 5.94 (o, 1H), 5.56 (s, 2H), 5.52 (o, 1H), 5.31 (o, 1H), 5.29 (o, 1H), 4.99 (m, 1H), 4.60 (s) and 4.66 (s) (2H), 4.56 (m, 1H), 4.14 (m, 1H), 3.85 (o, 1H), 3.78 (o, 2H), 3.65 (o, 2H), 3.64 (o, 1H), 3.64 (o, 1H), 3.60 (o, 28H), 3.56 (o, 2H), 3.54 (o, 2H), 3.47 (o, 1H), 3.36 (br o, 2H), 3.35 (o, 3H), 3.32 (o, 3H), 3.12 (o, 3H),3.09 (m, 1H), 2.83 (o) and 2.78 (o) (2H), 2.81 (o, 1H), 2.69 (m, 2H), 2.38 (o, 1H), 2.31 (o) and 1.75 (o) (2H), 2.30 (o, 1H), 2.07 (o, 2H), 2.04 (o) and 1.29 (o) (2H), 1.99 (o, 1H), 1.85 (o) and 1.54 (o) (2H), 1.83 (o, 1H), 1.77 (o, 2H), 1.71 (o) and 1.44 (o) (2H), 1.66 (o, 3H), 1.64 (o, 3H), 1.63 (o) and 1.73 (o) (2H), 1.62 (o) and 1.03 (o) (2H), 1.58 (o, 2H), 1.46 (o) and 1.19 (o) (2H), 1.36 (o, 1H), 1.31 (o, 2H), 1.25 (o) and 1.14 (o) (2H), 1.02 (o, 3H), 0.99 (o, 3H), 0.96 (o, 3H), 0.93 (o, 3H), 0.92 (o, 3H).

### EQUIVALENTS

While the present disclosure has been described in conjunction with the specific embodiments set forth above, many alternatives, modifications and other variations thereof will be apparent to those of ordinary skill in the art. All such alternatives, modifications and variations are intended to fall within the spirit and scope of the present disclosure.

1. A process for preparing a compound of formula (3), or a salt thereof, comprising:
(1a) contacting a compound of formula (1), or a salt thereof, 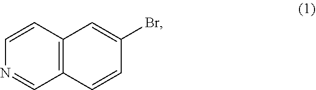 [SMILES: Brc1ccc2cnccc2c1]
with a reducing agent, to yield a compound of formula (2), or a salt thereof, 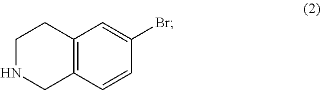 [SMILES: Brc1ccc2c(c1)CCNC2]
(2a) contacting a compound of formula (2), or a salt thereof, with an amino protecting group reagent to yield a compound of formula (3), or a salt thereof, 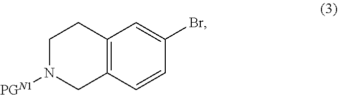
wherein PG^{N1} is an amino protecting group.

2. The process of claim 1, wherein the reducing agent is sodium borohydride, and/or wherein step (1a) is performed in the presence of acetic acid.

3. The process of claim 1, wherein:
(a) the amino protecting group reagent is triphenylmethyl chloride; and/or
(b) PG^{N1} is triphenylmethyl (trityl).

4. The process of claim 1, wherein:
(a) step (2a) is performed in the presence of an activating reagent, optionally wherein the activating reagent is 4-dimethylaminopyridine (DMAP); and/or
(b) step (2a) is performed in dichloromethane (DCM).

5. The process of claim 1, further comprising isolating the compound of formula (3).

6. The process of claim 1, further comprising:
(3a′) contacting the compound of formula (3), or a salt thereof, with an organometallic/metal reagent and formaldehyde to yield a compound of formula (5), or a salt thereof, 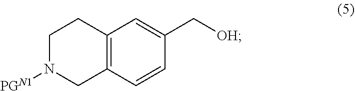
(3a) contacting the compound of formula (3), or a salt thereof, with an organometallic reagent and dimethylformamide (DMF) to yield a compound of formula (4), or a salt thereof, 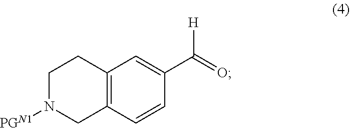
optionally wherein step (3a) is performed in tetrahydrofuran (THF), and/or wherein the organometallic reagent is an alkyl magnesium halide.

7. The process of claim 6, further comprising
(4a) contacting the compound of formula (4), or a salt thereof, with a reducing agent to yield a compound of formula (5), or a salt thereof, 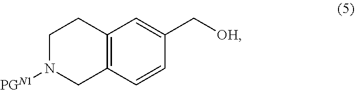
optionally wherein the reducing agent is sodium borohydride and/or wherein step (4a) is performed in a solvent selected from the group consisting of methanol, THF, and mixture thereof.

8. The process of claim 6, further comprising
(5a) contacting the compound of formula (5), or a salt thereof, with a PG^{N1} deprotecting reagent to yield a compound of formula (6), or a salt thereof, 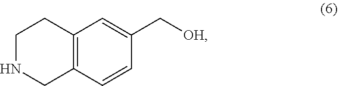 [SMILES: OCc1ccc2c(c1)CCNC2]
optionally wherein step (5a) is performed in DCM; and
(6a) contacting the compound of formula (6), or a salt thereof, with a Boc protecting group reagent to yield a compound of formula (7), or a salt thereof, 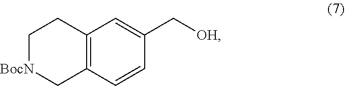 [SMILES: CC(C)(C)OC(=O)N1CCc2cc(CO)ccc2C1]
optionally wherein step (6a) is performed in THF, and/or wherein the process further comprises isolating the compound of formula (7).

9. The process of claim 8, wherein:
(a) the PG^{N1} deprotecting reagent is an acid; and/or
(b) the Boc protecting group reagent is Boc₂O.

10. The process of claim 8, further comprising
(7a) contacting the compound of formula (7), or a salt thereof, with an alcohol activating reagent to yield a compound of formula (8), or a salt thereof, 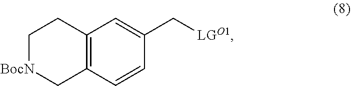
wherein −LG^{O1} is a leaving group,
optionally wherein the process further comprises isolating the compound of formula (8).

11. The process of claim 10, wherein:
(a) the alcohol activating reagent is a sulfonyl halide or a halogenating reagent, optionally methanesulfonyl chloride (mesyl chloride; CH₃SO₂Cl); and/or
(b) −LG^{O1} is a sulfonate ester or a halide, optionally mesylate (—O—SO₂CH₃).

12. The process of claim 10, wherein:
(a) step (7a) is performed in the presence of a base, optionally wherein the base is diisopropylethylamine (DIPEA); and/or
(b) step (7a) is performed in DCM.

13. The process of claim 10, further comprising
(8a) contacting the compound of formula (8), or a salt thereof, with a compound of formula (9), or a salt thereof, 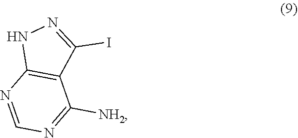 [SMILES: Nc1ncnc2[nH]nc(I)c12]
to yield a compound of formula (10), or a salt thereof, 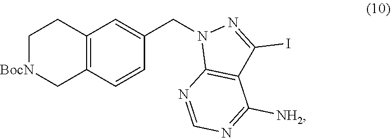 [SMILES: CC(C)(C)OC(=O)N1CCc2cc(Cn3nc(I)c4c(N)ncnc43)ccc2C1]
optionally wherein step (8a) is performed in DMF, and/or wherein the method further comprises isolating the compound of formula (10).

14. The process of claim 13, further comprising
(9a) contacting the compound of formula (10), or a salt thereof, with a compound of formula (11) or a salt thereof, 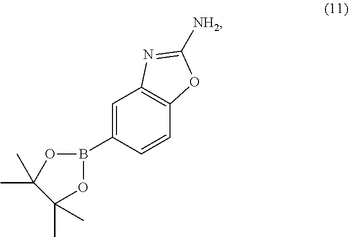 [SMILES: CC1(C)OB(c2ccc3oc(N)nc3c2)OC1(C)C]
to yield a compound of formula (12), or a salt thereof, 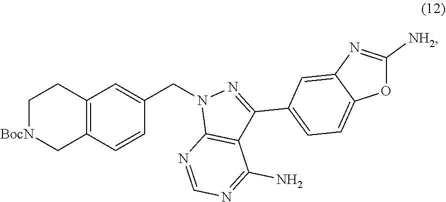 [SMILES: CC(C)(C)OC(=O)N1CCc2cc(Cn3nc(-c4ccc5oc(N)nc5c4)c4c(N)ncnc43)ccc2C1]
optionally wherein the process further comprises isolating the compound of formula (12).

15. The process of claim 14, wherein the compound of formula (11) is prepared by borylation of a compound of formula (11a), or a salt thereof, 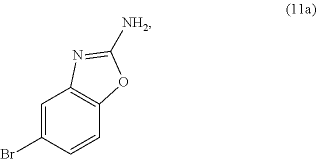 [SMILES: Nc1nc2cc(Br)ccc2o1]
optionally wherein borylation is performed with contact with a boronic ester reagent, further optionally wherein the boronic ester reagent is bis(pinacolato)diboron (B₂Pin₂).

16. The process of claim 14, wherein:
(a) step (9a) is performed in the presence of a palladium catalyst, optionally wherein the palladium catalyst is Pd(PPh₃)₄; and/or
(b) step (9a) is performed in a solvent selected from the group consisting of water, dioxane, and mixture thereof.

17. The process of claim 14, further comprising
(10a) contacting the compound of formula (12) with an acid to yield a compound of formula (13), 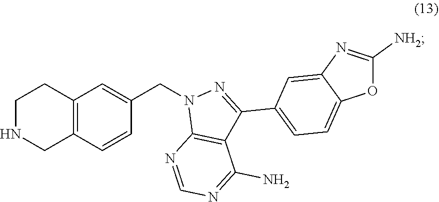 [SMILES: Nc1nc2cc(-c3nn(Cc4ccc5c(c4)CCNC5)c4ncnc(N)c34)ccc2o1]
(11a) preparing a salt of a compound of formula (13);
optionally wherein step (10a) and step (11a) are performed in water.

18. The process of claim 17, wherein:
(a) the acid is hydrochloric acid, thereby yielding a hydrochloric salt of compound of formula (13a), 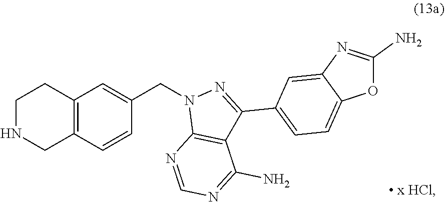
wherein x is 1, 2, or 3; or
(b) the acid is trifluoroacetic acid, thereby yielding a TFA salt of compound of formula (13c), 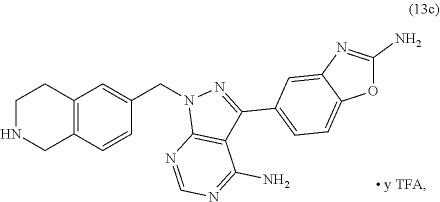
wherein y is 1, 2, or 3.

19. The process of claim 17, further comprising isolating the compound of formula (13), (13a), or (13c).

20. A process for preparing a compound of formula (21), or a salt thereof, comprising:
(1b) contacting a compound of formula (20), or a salt thereof, 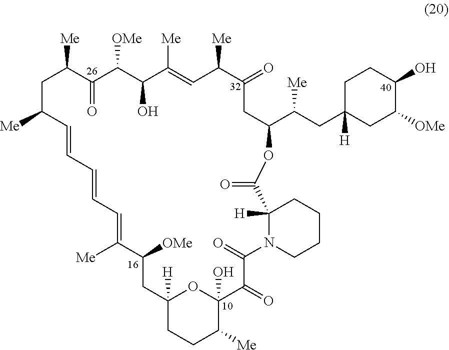 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)CC(=O)[C@H](C)/C=C(\C)[C@@H](O)[C@@H](OC)C(=O)[C@H](C)C[C@H](C)/C=C/C=C/C=C/1C]
with a hydroxyl protecting group reagent, to yield a compound of formula (21), or a salt thereof, 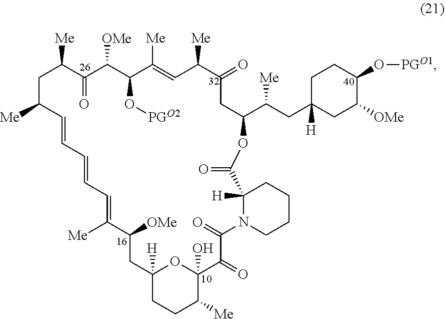
wherein PG^{O1} and PG^{O2} are the same or different at each instance a hydroxyl protecting group, optionally wherein step (1b) is performed in the presence of imidazole, and/or wherein step (1b) is performed in DCM.

21. The process of claim 20, wherein:
(a) each hydroxyl protecting group reagent is triethylchlorosilane (TES-Cl);
(b) PG^{O1} is triethylsilyl ether (TES); and/or
(c) PG^{O2} is triethylsilyl ether (TES).

22. The process of claim 20, further comprising isolating the compound of formula (21).

23. The process of claim 20, further comprising:
(2b) contacting a compound of formula (21), or a salt thereof, with a reducing agent to yield a compound of formula (22), or a salt thereof, 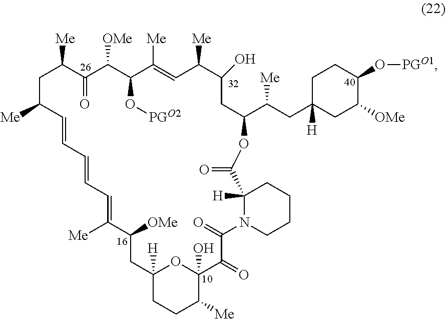
optionally wherein the reducing agent is LiAl(Ot-Bu)₃H, and/or wherein step (2b) is performed in THF.

24. The process of claim 23, wherein the product from step (2b) is subsequently contacted with Cu(OAc)₂.

25. The process of claim 23, further comprising isolating the compound of formula (22).

26. The process of claim 23, further comprising:
(3b) contacting a compound of formula (22), or a salt thereof, with a PG^{O1}deprotecting reagent and a PG^{O2} deprotecting reagent to yield a compound of formula (23), or a salt thereof, 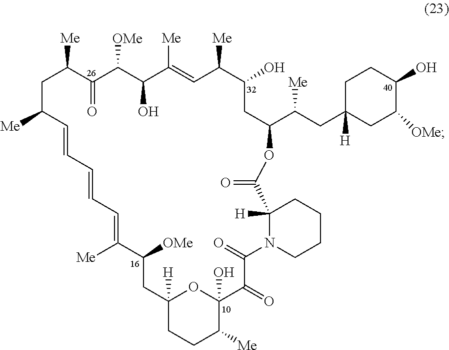 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](O)[C@H](OC)C2)C[C@@H](O)[C@H](C)/C=C(\C)[C@@H](O)[C@@H](OC)C(=O)[C@H](C)C[C@H](C)/C=C/C=C/C=C/1C]
optionally wherein step (3b) is performed in THF, further optionally wherein the PG^{O1} deprotecting reagent is an acid, or wherein the PG^{O2} deprotecting reagent is an acid.

27. The process of claim 26, further comprising isolating the compound of formula (23).

28. The process of claim 26, further comprising
(4b) contacting the compound of formula (23), or a salt thereof, with a compound of formula (24), or a salt thereof, 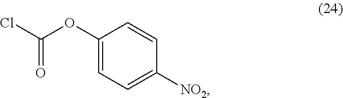 [SMILES: O=C(Cl)Oc1ccc([N+](=O)[O-])cc1]
to yield a compound of formula (25), or a salt thereof, 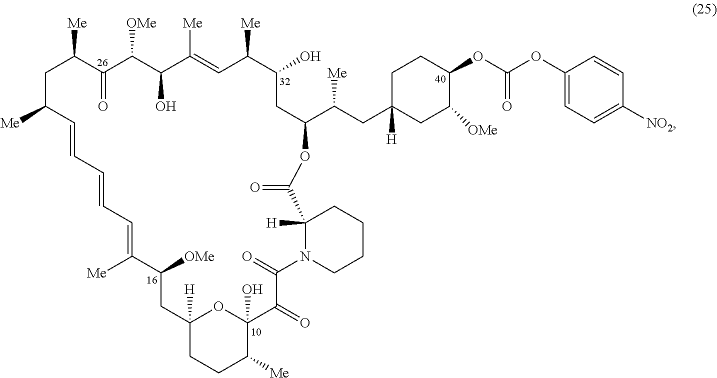 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]
optionally wherein step (4b) is performed in DCM, and/or wherein the process further comprises isolating the compound of formula (25).

29. A process for preparing a compound of formula (31), or a salt thereof, comprising:
(1c) contacting a compound of formula (30), or a salt thereof, 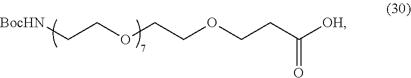
with a compound of formula (13), or a salt thereof, 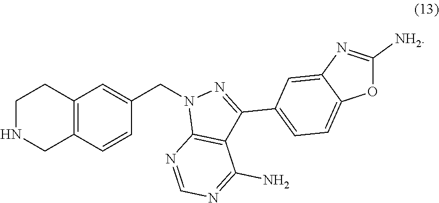 [SMILES: Nc1nc2cc(-c3nn(Cc4ccc5c(c4)CCNC5)c4ncnc(N)c34)ccc2o1]
to yield the compound of formula (31), or a salt thereof, 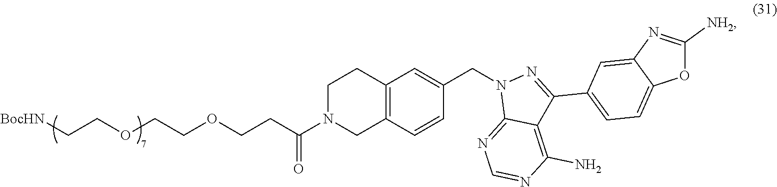
optionally wherein the process further comprises isolating the compound of formula (31).

30. The process of claim 29, wherein the compound of formula (13), or a salt thereof, is:
(a) a compound of formula (13a), 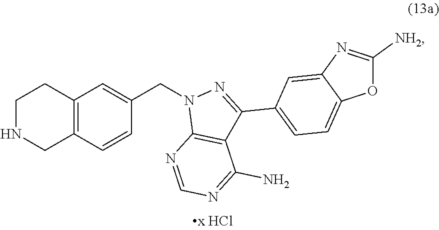
wherein x is 1, 2, or 3; or
(b) a compound of formula (13c), 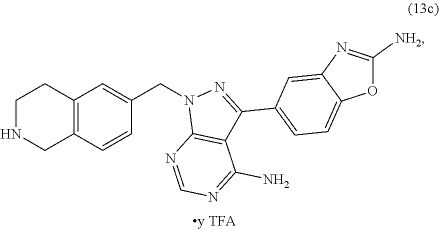
wherein y is 1, 2, or 3.

31. The process of claim 29, wherein:
(a) step (1c) is performed in the presence of a coupling reagent, optionally wherein the coupling reagent is 1-ethyl-3-(3-dimethylaminopropyl)carbodiimide (EDCI);
(b) step (1c) is performed in the presence of an activating reagent, optionally wherein the activating reagent is hydroxybenzotriazole (HOBt); and/or
(c) step (1c) is performed in dimethylacetamide (DMM).

32. The process of claim 29, further comprising
(2c) contacting the compound of formula (31) with a Boc removing agent to yield a compound of formula (32), or a salt thereof, 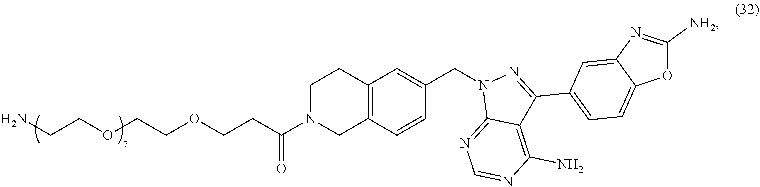
optionally wherein the Boc removing reagent is hydrochloric acid, and/or wherein step (2c) is performed in a solvent selected from the group consisting of water, DCM, dimethylacetamide (DMAc), and mixtures thereof.

33. The process of claim 32, further comprising isolating the compound of formula (32).

34. The process of claim 32, further comprising
(3c) contacting the compound of formula (32), or a salt thereof, with a compound of formula (25), or a salt thereof, 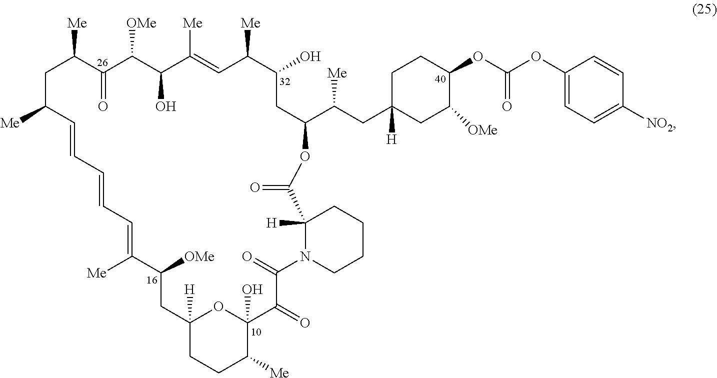 [SMILES: CO[C@H]1C[C@@H]2CC[C@@H](C)[C@@](O)(O2)C(=O)C(=O)N2CCCC[C@H]2C(=O)O[C@H]([C@H](C)C[C@@H]2CC[C@@H](OC(=O)Oc3ccc([N+](=O)[O-])cc3)[C@H](OC)C2)C[C@@H](O)[C@H](C)/C=C(\C)[C@@H](O)[C@@H](OC)C(=O)[C@H](C)C[C@H](C)/C=C/C=C/C=C/1C]
to yield a compound of formula (33), or a salt thereof, 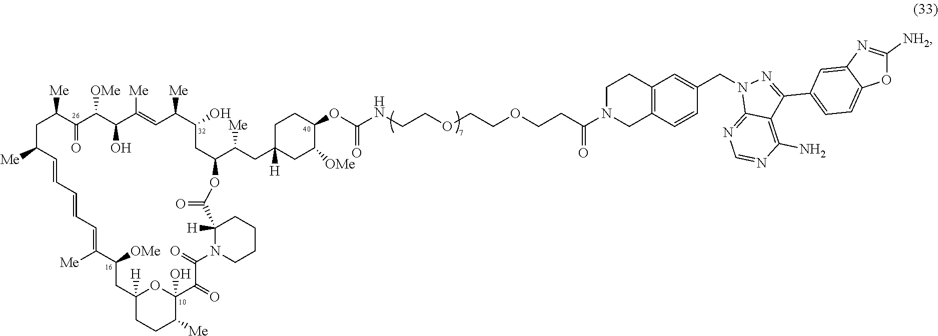
optionally wherein step (3c) is performed in DMAc, and/or wherein the process further comprises isolating the compound of formula (33).

35. A compound of:
(a) formula (13), or a salt thereof, 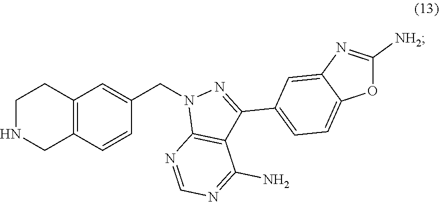 [SMILES: Nc1nc2cc(-c3nn(Cc4ccc5c(c4)CCNC5)c4ncnc(N)c34)ccc2o1]
(b) formula (13a), 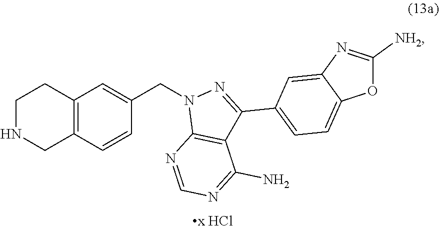
wherein x is 1, 2, or 3;
(c) formula (13c), 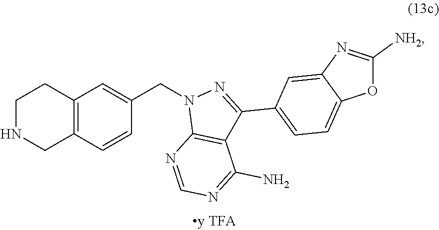
wherein y is 1, 2, or 3; or
(d) formula (32), or a salt thereof, [chemical structure]
